SEQUENCE LISTING (35 sequences)

SEQ ID NO: 1 - DNA, 18 bp, Artificial
  Forward primer for transforming growth factor-beta
caggcgtcagcgtattcc

SEQ ID NO: 2 - DNA, 18 bp, Artificial
  Reverse primer for transforming growth factor-beta
ccttccctacccgtccaa

SEQ ID NO: 3 - DNA, 20 bp, Artificial
  Forward primer for interferon gamma
actggcaaaaggatggtgac

SEQ ID NO: 4 - DNA, 20 bp, Artificial
  Reverse primer for interferon gamma
tgagctcattgaatgcttgg

SEQ ID NO: 5 - DNA, 24 bp, Artificial
  Forward primer for major histocompatibility complex class II
gtctcagactgtaagacctgaatg

SEQ ID NO: 6 - DNA, 22 bp, Artificial
  Reverse primer for major histocompatibility complex class II
gctgaggtggtggatacaatag

SEQ ID NO: 7 - DNA, 21 bp, Artificial
  Forward primer for interleukin 12
caatcacgctacctcctcttt

SEQ ID NO: 8 - DNA, 21 bp, Artificial
  Reverse primer for interleukin 12
accatgtcatctgtggtcttc

SEQ ID NO: 9 - DNA, 20 bp, Artificial
  Forward primer for mothers against decapentaplegic homolog 2
tcacagacccatcaaactcg

SEQ ID NO: 10 - DNA, 19 bp, Artificial
  Reverse primer for mothers against decapentaplegic homolog 2
actcagcaaacacttcccc

SEQ ID NO: 11 - DNA, 21 bp, Artificial
  Forward primer for estrogen receptor 1
aaccgcccatgatctattctg

SEQ ID NO: 12 - DNA, 20 bp, Artificial
  Reverse primer for estrogen receptor 1
agattcaagtccccaaagcc

SEQ ID NO: 13 - DNA, 22 bp, Artificial
  Forward primer for estrogen receptor 2
acgaagtaggaatggtcaagtg

SEQ ID NO: 14 - DNA, 21 bp, Artificial
  Reverse primer for estrogen receptor 2
ggttctcttggctttgttcag

SEQ ID NO: 15 - DNA, 21 bp, Artificial
  Forward primer for interferon regulatory factor 1
gaagggaagatagccgaagac

SEQ ID NO: 16 - DNA, 19 bp, Artificial
  Reverse primer for interferon regulatory factor 1
tctggttcctctttgcagc

SEQ ID NO: 17 - DNA, 23 bp, Artificial
  Forward primer for interferon regulatory factor 2
gatttctcctgtgtcttcctacg

SEQ ID NO: 18 - DNA, 18 bp, Artificial
  Reverse primer for interferon regulatory factor 2
ttccgtgtccccatgttg

SEQ ID NO: 19 - DNA, 22 bp, Artificial
  Forward primer for lymphoid enhancer-binding factor
agaacaccctgatgaaggaaag

SEQ ID NO: 20 - DNA, 21 bp, Artificial
  Reverse primer for lymphoid enhancer-binding factor
gtacgggtcgctgttcatatt

SEQ ID NO: 21 - DNA, 18 bp, Artificial
  Forward primer for transcription factor
ggttcacccacccatcct

SEQ ID NO: 22 - DNA, 18 bp, Artificial
  Reverse primer for transcription factor
ttgcgggccagttcatag

SEQ ID NO: 23 - DNA, 20 bp, Artificial
  Forward primer for axis inhibition protein 2
agcctaaaggtcttatgtgg

SEQ ID NO: 24 - DNA, 18 bp, Artificial
  Reverse primer for axis inhibition protein 2
atggaatcgtcggtcagt

SEQ ID NO: 25 - DNA, 20 bp, Artificial
  Forward primer for wnt-7a
ggacgagtgtcagtttcagt

SEQ ID NO: 26 - DNA, 18 bp, Artificial
  Reverse primer for wnt-7a
cacagtcgctcaggttgc

SEQ ID NO: 27 - DNA, 39 bp, Artificial
  Forward primer for mutagenesis
aatgtcgcgggcggaggctgacccggatcttgaagcact

SEQ ID NO: 28 - DNA, 39 bp, Artificial
  Reverse primer for mutagenesis
aagtgcttcaagatccgggtcacctccgcccgcgacatt

SEQ ID NO: 29 - DNA, 21 bp, Artificial
  Forward primer for mutant
cctcaagttcaaggaccaaca

SEQ ID NO: 30 - DNA, 22 bp, Artificial
  Reverse primer for mutant
gctgtgaaggagagcaagatta

SEQ ID NO: 31 - RNA, 23 nt, Artificial
  miR-17 mimic
caaagugcuuacagugcagguag

SEQ ID NO: 32 - RNA, 23 nt, Homo sapiens
uggaagacuagugauuuuguugu

SEQ ID NO: 33 - RNA, 23 nt, Mus musculus
gauagacgugaucuacguggaau

SEQ ID NO: 34 - RNA, 20 nt, Mus musculus
cgggcugcuucugcaccuua

SEQ ID NO: 35 - RNA, 20 nt, Artificial
  lrf2 mutant
cgggcggaggcugacccgga

(19) United States
(12) Patent Application Publication
(10) Pub. No.: US 2018/0362974 A1
(43) Pub. Date: Dec. 20, 2018

(54) EDIBLE PLANT-DERIVED MICROVESICLE COMPOSITIONS FOR DELIVERY OF miRNA AND METHODS FOR TREATMENT OF CANCER

(71) Applicant: UNIVERSITY OF LOUISVILLE RESEARCH FOUNDATION, INC., Louisville, KY (US)

(72) Inventor: Huang-Ge ZHANG, Louisville, KY (US)

(21) Appl. No.: 15/740,591

(22) PCT Filed: Jul. 1, 2016
(86) PCT No.: PCT/US2016/040710

§ 371 Date: Dec. 28, 2017

Related U.S. Application Data
(60) Provisional application No. 62/188,361, filed on Jul. 2, 2015.

(51) Int. Cl. C12N 15/113; A61K 9/127

(52) U.S. Cl. CPC C12N 15/113; C12N 2320/32; C12N 2310/141; A61K 9/127

(57) ABSTRACT

Microvesicle compositions and methods of use thereof are provided. The microvesicle composition includes a miRNA encapsulated by a microvesicle, wherein the microvesicle is derived from an edible plant. The method of use thereof includes treating a cancer in a subject by administering to the subject an effective amount of a microvesicle composition.

### RELATED APPLICATIONS

This application claims priority to U.S. Provisional Application Ser. No. 62/188,361, filed Jul. 2, 2015, which is incorporated herein by this reference.

### GOVERNMENT INTEREST

This invention was made with government support under grant no. UH2TR000875 awarded by the National Institutes of Health. The government has certain rights in the invention.

### TECHNICAL FIELD

The presently-disclosed subject matter relates to edible plant-derived microvesicle compositions for the delivery of miRNA and methods of using the same for the treatment of cancer. In particular, the presently-disclosed subject matter relates to compositions that include miRNAs encapsulated by edible plant-derived microvesicles and that are useful in the diagnosis and treatment of cancer.

### BACKGROUND

Microvesicles are small assemblies of lipid molecules (50-1000 nm in size), which include, but are not limited to, exosomes, epididimosomes, argosomes, exosome-like vesicles, microparticles, promininosomes, prostasomes, dexosomes, texosomes, dex, tex, archeosomes, and oncosomes. Microvesicles can be formed by a variety of processes, including the release of apoptotic bodies, the budding of microvesicles directly from the cytoplasmic membrane of a cell, and exocytosis from multivesicular bodies. For example, exosomes are commonly formed by their secretion from the endosomal membrane compartments of cells as a consequence of fusion of multivesicular bodies with the plasma membrane. The MVBs are formed by inward budding from the endosomal membrane and subsequent pinching off of small vesicles into the luminal space. The internal vesicles present in the MVBs are then released into the extracellular fluid as so-called exosomes.

In addition to being formed by a variety of processes, microvesicles are produced by a variety of eukaryotic cells, including plant cells, and the release and uptake of these secreted membrane vesicles has been shown to allow for the transfer of small packages of information (bioactive molecules) to numerous target cells. Indeed, the contents of these packages are enriched in proteins, lipids, and microRNAs, and recent biological and proteomic studies of microvesicles have further revealed the biological functions of microvesicles. From these studies, it appears that one of the major roles of microvesicles is the exchange of information through their secretion, with the functional consequences of such membrane transfers including the induction, amplification and/or modulation of recipient cell function. In this regard, a number of studies have led to the idea that microvesicles are a common mode of intercellular communication.

Despite the number of studies linking microvesicles to intracellular communication, however, to date, the use of microvesicles as an efficient and effective delivery vehicle has yet to be fully realized due, at least in part, to the inability to produce the large quantities of microvesicles that are needed for therapeutic applications and to the inability to effectively and efficiently utilize the microvesicles to deliver a therapeutic agent to target cells and tissues, while also retaining the biological activity of the therapeutic agents.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently disclosed subject matter includes microvesicle compositions and methods of use thereof. In some embodiments, the presently-disclosed subject matter relates to a composition including a miRNA encapsulated by a microvesicle. In some embodiments, the microvesicle is derived from an edible plant. For example, in one embodiment, the edible plant includes a fruit, such as, but not limited to, a grape, a grapefruit, and/or a tomato. In some embodiments, the miRNA includes miR18a, miR17, or a combination thereof.

Additionally, in some embodiments, the microvesicle includes a cancer targeting moiety for directing the composition to a cancer cell. One suitable cancer targeting moiety includes, but is not limited to, folic-acid. In some embodiments, the microvesicle comprises a nanovector hyrided with polyethylenimine. In one embodiment, the nanovector includes a grapefruit-derived nanovector. In another embodiment, the nanovector decreases a toxicity of the polyethylenimine.

In some embodiments, the composition is a pharmaceutical composition including an edible plant-derived microvesicle, a miRNA encapsulated by the microvesicle, and a pharmaceutically-acceptable vehicle, carrier, or excipient.

In some embodiments, the presently-disclosed subject matter relates to a method for treating cancer in a subject. For example, in one embodiment, the method for treating cancer includes administering to a subject an effective amount of a composition including a miRNA encapsulated by a microvesicle derived from an edible plant. In some embodiments, the method includes treating cancer such as, but not limited to, brain cancer, liver cancer, colon cancer, or a combination thereof. Additionally or alternatively, the method may include treating liver metastases. The composition may be administered by any suitable route of administration, including, but not limited to, orally and/or intranasally.

### BRIEF DESCRIPTION OF THE DRAWINGS

FIG. 1A includes representative images of sucrose banded GNVs, pGNV/RNA, and FA-pGNV/RNA visualized and imaged by electron microscopy.

FIG. 1B includes graphs illustrating zeta potential and size distribution of GNVs, pGNV/RNA, and FA-pGNV/RNA. The zeta potentials were analyzed using a ZetaSizer.

FIGS. 2A-2B are images and graphs illustrating how intranasal administration of GNVs results in localization to the brain. DIR-labeled GNVs (green) or controls were administered intranasally into C57BL/6j mice. 12 h post-intranasal administration, the brain was cut sagittally or coronally for imaging using the Odyssey laser-scanning imager. (FIG. 2A) Representative images from the center of the brain (n=5) are shown. (FIG. 2B) Graph illustrating fluorescent intensity of GNVs-DIR and DOTAP-DIR in the olfactory bulb, cerebral cortex and striatum, hippocampus and thalamus, and cerebellum. Results were obtained from three independent experiments with five mice in each group of mice.

FIGS. 3A-3E are images and graphs illustrating that pGNVs have a better capacity for carrying RNA without toxicity. RNA loaded pGNVs (pGNV/RNA) and PEI-RNA were purified by ultracentrifugation. (FIG. 3A) Sucrose banded pGNV/RNA and PEI-RNA were visualized and imaged by electron microscopy. (FIG. 3B) Size distribution (top panel) and Zeta potential (bottom panel) of pGNV/RNA) or PEI/RNA were analyzed using a ZetaSizer. (FIG. 3C) Loading efficiency of miR17-Dy547 was determined using a fluorescence microplate reader (EX/Em=530/590 nm) and expressed as %=(miR17-DY547 in pGNV/RNA or GNVs)/Total RNA used for loading×100%. Images (a, b) and data (c, n=5) are representative of at least three independent experiments. d. Intranasal administration of pGNV/RNA-Syto60 results in localization to the brain. Syto60-labeled RNA (20 μg, red) carried by DIR labeled pGNVs (green) was administered intranasally to C57BL/6j mice. At different timepoints post-intranasal administration, the brain was cut sagittally, and the ventral sides of cut brain were placed against the scanner for imaging using the Odyssey laser-scanning imager. Enlarged images are shown at the bottom. e. DIR labeled GNVs or pGNV/RNA was administered intranasally to C57BL/6j mice. At 12 h post-intranasal administration, the brain was cut sagittally, and the ventral sides of cut brain were placed against the scanner for imaging using the Odyssey laser-scanning imager. (FIG. 3D-3E) Representative sagittal images from the center of the brain (n=5). pGNV/RNA or PEI/RNA were administered intranasally to C57BL/6j mice. Mice were sacrificed 12h or 24 h after intranasal administration of pGNV/RNA or PEI/RNA. C57BL/6j mice were intraperitoneally (i.p.) injected with bacterial lipopolysaccharide (2.5 mg/kg) or PBS as a control and sacrificed at 12h and 24 h post-injection as a control. Brain tissue sections were fixed as described in the Materials and Methods section. Frozen sections (30 μm) of the anterior part of the brain were stained with the antimicroglial cell marker Iba-1 (green color) or macrophages (red). Slides were examined and photographed using microscope with an attached camera (Olympus America, Center Valley, Pa.). Each photograph is representative of three different independent experiments (n=5). Original magnifications ×40.

FIG. 4 includes graphs and images illustrating GNVs, GNV/RNA-syto60, and pGNV/RNA-syto60 samples run on a discontinuous sucrose gradient, and sucrose banded samples as indicated by arrows were collected and sucrose density was determined using a densitometer. The RNA associated with each banded sample was quantitatively analyzed with a fluorescent meter at Ex/Em=530/590 nm (Syto60), and expressed as pg/μ1 of sample collected.

FIG. 5 includes graphs illustrating UV-vis absorption spectrum of PEI-RNA and pGNV/RNA complex (left panel) and standard curve of PEI (right panel).

FIG. 6 includes representative images from the cultured GL-26-luc cells illustrating expression of folate receptor (n=3). GL-26 cells were cultured for 24 hour and the expression of folate receptor on the cells was detected by staining with anti-folate receptor antibody and isotype IgG1 was used as a control.

FIGS. 7A-7D are graphs and images illustrating folate receptor mediated uptake of FA-pGNVs. GL-26-luc cells were cultured in the presence of Dylight547 labeled miR17 or Syto60 labeled RNA carried by FA-pGNVs (FA-pGNV/miR17-Dy547, FA-pGNV/RNA-Syto60) or by pGNVs (pGNV/miR17-DY547, pGNV/RNA-Syto60). (FIG. 7A) Representative images of cells (n=3) were taken at 2 h after addition of FA-pGNVs/miR17-Dy547, pGNVs/miR17-DY547, FA-pGNV/RNA-Syto60, or pGNV/RNA-Syto60 using a confocal microscope at a magnification of ×200 (top panel) or ×600 (bottom panel), and quantified by counting the number of DY547⁺ cells in five individual fields in each well. % of DY547⁺ cells was calculated based on the number of DY547⁺ cells/numbers of FR⁺ cells ×100. The results are presented as the mean±S.E.M.; **P<0.01. (FIG. 7B) At different time points, post incubation at 37° C., transfection efficiency of FA-pGNV/miR17-Dy547 or pGNV/miR17-DY547 was analyzed by measuring fluorescent density using a microplate reader at Ex/Em=530 nm/590 nm. (FIG. 7C), before adding to GL-26-luc cultures, FA-pGNV/miR17-Dy547 (100 nmole) was pre-mixed with different concentrations of folic acid and then GL-26-luc cells were cultured in the presence of folic acid premixed with FA-pGNV/miR17-Dy547 for 2 h. The effects of folate on the transfection efficiency of FA-pGNV/miR17-Dy547 was analyzed by measuring fluorescent density using a microplate reader at Ex/Em=530 nm/590 nm. Data (FIGS. 7A-C) are the mean±S.E.M. of two experiments (n=5). (FIG. 7D), FA-pGNV/miR17-Dy547 more efficiently targeted brain tumor. 2×10⁴ GL26-luc cells per mouse were injected intra-cranially in 6-week-old wild-type B6 mice. Five-day tumor-bearing mice were then treated intranasally with FA-pGNV/miR17-Dy547 in PBS or pGNV/miR17-Dy547. FA-pGNV/miR17-Dy547 in PBS or pGNV/miR17-Dy547 (red representing miR17 labeled with Dylight 547, 20 μg of miR17 carried by pGNVs) was administered intranasally into C57BL/6j mice. Results of hematoxylin and eosin (HE) staining showing tumor tissue as indicated by arrows (top panel). DIR dye labeled FA-pGNV/miR17-Dy547 or pGNV/miR17-Dy547 (second panel from the top, the results represent the mean±S.E.M. of three independent experiments, bar graph). H.E. stained brain sections of GL-26-luc tumor bearing mice (the first column from right) or miR17-Dy547 (red) or anti-folate receptor (FR) antibody stained (green) brain tumor sections and adjacent area of mice treated with the agents listed (third and fourth panel from the top). Original magnification ×20. Data represent at least three experiments with five mice/group.

FIGS. 8A-8E are graphs and images illustrating that FA-pGNV/miR17-Dy547 treatment prevents the growth of in vivo injected brain tumor cells. 2×10⁴ GL26-luc cells per mouse were injected intra-cranially in 6-week-old wild-type B6 mice. Fifteen-day tumor-bearing mice were then treated intranasally on a daily basis with FA-pGNVs/siRNA-luc or FA-pGNVs/siRNA scramble control. The mice were imaged on the hours as indicated in FIG. 8A. (FIG. 8A) Representative photograph of brain tumor signals of a mouse from each group (n=5) is shown (left). The luciferase activity of injected GL26-Luc cells was determined by photon emissions of mice treated at 24 or 48 divided by at 0 h (right). The results are based on two independent experiments with data pooled for mice in each experiment (n=5) and are presented as the mean±S.E.M.; **P<0.01. (FIG. 8B) Mice were intra-cranially injected with GL26-luc and treated every three day for 21 days beginning on day 5 after tumor cells were implanted. The mice were imaged on the days as indicated in the labeling of FIG. 8B. A representative photograph of brain tumor signals of a mouse from each group (n=5) is shown (left). The luciferase activity of injected GL26-Luc cells was determined by dividing photon emissions of mice treated at day indicated as labeled in “X” axis by the photon emissions of mice treated at day 0 (right). The results are based on two independent experiments with data pooled for mice in each experiment (n=5) and are presented as the mean±S.E.M.; *P<0.05, **P<0.01. (FIG. 8C) Percent of FA-pGNVs/miR17, FA-pGNVs/scramble miRNA or PBS mice surviving was calculated. One representative experiment of 4 independent experiments is shown (n=5 females per group). Results of anti-luciferase and MHCI staining (FIG. 8D), or anti-luciferase/MHCl/DX5 staining (FIG. 8E) of brain tumor sections and adjacent area of mice treated with the agents listed. Original magnification x20. Data represent at least three experiments with five mice/group

FIG. 9 includes a graph illustrating quantitative real-time PCR (qRT-PCR) analysis of miR17 from total RNA extracted from transfected GL26-luc cells. Relative quantification of miR17 in treated GL26-luc cells versus untreated GL26-luc cells (Naive) was performed using a CFX96 Realtime System (Bio-Rad Laboratories, Hercules, Calif.) and SsoFast Evagreen supermixture (Bio-Rad Laboratories), according to the manufacturers' instructions.

FIG. 10 includes graphs illustrating reduction of MHC class I on GL26-luc tumor cells by miR-17 encapsulated in FA-pGNVs. The expression levels of MHC class I in FA-pGNVs and FA-pGNV/miR17 treated GL26-luc cell line were analyzed by flow cytometry. Representative images from the cultured GL-26-luc cells are shown (n=5).

FIG. 11 includes an image illustrating sucrose-banded particles from grapefruit juice. The nanoparticles were isolated from grapefruit juice by sucrose gradient (8,30, 45, and 60% sucrose in 20mM Tri-Cl, pH 7.2). Particles from band 2 were used for preparation of GNVs.

FIGS. 12A-12C include graphs illustrating optimizing conditions for GNVs encapsulating RNA. (FIG. 12A) Effects of ultraviolet (UV) radiation at 0, 250, 500, 1000, 2000 millijoule per square centimeter (mJ/cm²) on size distribution of GNVs analyzed using the Zetasizer Nano ZS. (FIG. 12B) Quantitatively analysis of the effects of ultraviolet (UV) radiation on GNVs size distribution (red) and the efficiency of packing RNA into GNVs (blue). Efficiency of RNA encapsulated in GNVs was defined as the amount of RNA isolated from GNVs divided by amount of RNA added before GNVs were assembled. (FIG. 12C) Evaluation of GNVs packing efficiency of RNAs pre-dissolved in H₂O, PBS (pH 7.4), and NaCl (155 mM). *P<0,05 and **P<0.01 (two-tailed t-test). Data are representative of three independent experiments (n=3, error bars, s.e.m.).

FIGS. 13A-13F Characteristics and biological activity of optimized GNVs (OGNVs) encapsulating RNA (FIG. 13A) Size distribution of GNVs analyzed using the Zetasizer Nano ZS. GNVs encapsulating RNA pre-dissolved in H₂O, PBS (pH 7.4), and NaCl (155 mM). (FIG. 13B) Quantification of size distribution of GNVs encapsulating RNA pre-dissolved in H₂O, PBS (pH 7.4), and NaCl (155 mM). (FIG. 13C) Surface charge of GNVs encapsulating RNA pre-dissolved in H₂O, PBS (pH 7.4), NaCl (155 mM) analyzed using the Zetasizer Nano ZS (left). Quantification of GNV surface charge (right). (FIG. 13D) 200 nM of GNVs encapsulating 20 of total RNA pre-dissolved in NaCl (155mM) and subsequently exposed to UV radiation (500 mJ/cm²). Distribution of PKH67-labeled (green) OGNVs and Exo-GLOW-labeled (red) RNAs were visualized using a confocal microscopy. (FIG. 13E) Fluorescence intensity of Exo-GLOW-labeled RNAs encapsulated in OGNVs or PEI was measured by a Biotek Synergy HT plate reader (460 nm excitation, 420 nm emission). Nanoparticles were made with OGNVs or polyethylenimine (PEI, 0.2 ng/μl ) with/without encapsulated RNA in the presence/absence UV (500 mJ/cm²) exposure. (FIG. 13F) Assessment of luciferase activity using the Dual-Luciferase Reporter Assay System (Promega) for the U87MG stably expressing firefly luciferase transfected with OGNVs or OGNVs encapsulating luciferase siRNA (si-Luci). *P<0.05 and **P<0.01 (two-tailed t-test). Data are representative of three independent experiments (n=3, error bars, s.e.m.).

FIG. 14 includes an image illustrating RNase digestion of RNA and OGNV RNA.

FIGS. 15A-15E include graphs and images illustrating that OGNV-mediated delivery of miRNA is taken up by mouse Kupffer cells in vivo. (FIG. 15A) PKH26-labeled (red) OGNVs located in liver Kupffer cells (F4/80⁺, green), not in spleen macrophages (F4/80⁺, green) from BALB/c mice are visualized with confocal microscopy, assessed 1 h and 24 h after intravenous injection. (FIG. 15B) Analysis of Alexa Fluor fluorescent streptavidin conjugates with confocal microscope, assessed 24 h after intravenous injection of OGNVs alone, OGNVs with biotin-conjugated miR-18a (bio-miR-18a), or bio-miR-18a alone. (FIG. 15C) Frequency of F4/80⁺ cells and PKH26-labled OGNVs in the liver from BALB/c mice assessed using flow cytometry. Numbers in quadrants indicate percent cells in each. (FIG. 15D) Quantification of miR-18a level in leukocytes from BALB/c mouse liver and spleen assessed 24 h after intravenous injection of OGNVs with miR-18a by quantitative real-time PCR (qPCR). *P<0.05 and **P<0.01 (two-tailed t-test). Data are representative of three independent experiments (error bars, S.E.M.). (FIG. 15E) Expression of miR-18a in hepatocytes from naïve BALB/c mice, CT26 liver metastasis mice with OGNVs/Ctrl or OGNVs/miR-18a treatment assessed by quantitative real-time PCR (qPCR).

FIGS. 16A-16H include graphs and images illustrating that miR-18a encapsulated in OGNVs inhibits liver metastasis of colon cancer and induces Kupffer cell polarization into M1. (FIG. 16A) Schematic representation of the treatment schedule. All groups of mice were euthanized 14 days after the intra-splenic tumor inoculation, and tumor specimens were obtained for analysis. (FIG. 16B) Frequency of MHCII, TGFIβ, IL-12, IFNγ, CD80, CD86, CD206, and IL-10 positive cells in liver F4/80⁺ cells from naïve BALB/c mice, CT26 liver metastasis mice treated with OGNVs packing control miRNA (OGNVs/Ctrl) or OGNVs packing miR-18a (OGNVs/miR18a) assessed by flow cytometry. (FIG. 16C) The histogram shows the quantification of results at (FIG. 16B). (FIG. 16D) Expression of mature miR-18a, MHCII, TGFIβ, IL-12, IFNγ, and iNOS in liver F4/80⁺ cells was assessed by qPCR. (FIG. 16E) Representative livers (up) and representative hematoxylin and eosin (H&E)-stained sections of livers (middle, 20×; bottom, 400× magnification). (FIG. 16F) Liver weight (left) and liver metastatic nodule number and size (right). (FIG. 16G) Survival of mice after intra-splenic injection of CT26 cells. (FIG. 16H) Frequency of IFNγ⁺ cells in liver CD3⁺Dx5⁺ (NKT) cells, CD3⁻Dx5⁺ (NK) cells, and CD3⁺Dx5⁻ (T) cells. Right, quantification of results; each symbol represents an individual mouse. *P<0.05 (two-tailed t-test). Data are representative of three independent experiments (error bars, S.E.M.).

FIG. 17 includes graphs illustrating induction of IFNγ⁻NK and IFNγ⁺NKT by OGNVs-miR-18a. Frequency of IFNγ⁺ cells in liver CD3⁺Dx5⁺ (NKT) cells, CD3⁻Dx5⁺ (NK) cells, and CD3⁺Dx5⁻ (T) cells from CT26 liver metastasis mice treated with OGNVs-Ctrl, OGNVs-miR-18a with/without IL-12 siRNA knockdown assessed by flow cytometry (Left); quantification of FACS analyzed results; each symbol represents an individual mouse (Right).

FIGS. 18A-B include graphs illustrating IFNγ and IL-12 levels in various cells. (FIG. 18A) Expression of IFNγ in various cells. (FIG. 18B) IL-12 levels in various cells.

FIGS. 19A-19E include graphs and images illustrating that depletion of macrophages restricted the response of miR-18a against liver metastasis. (FIG. 19A) Schematic representation of treatment schedule. All groups of mice were euthanized 14 days after the intra-splenic tumor injection, and tumor specimens were obtained for analysis. (FIG. 19B) Frequency of F4/80⁺ cells in liver leukocytes from clodronate treated (110 mg/kg) mice, with or without RAW264.7 cells assessed by flow cytometry. (FIG. 19C) PKH26-labeled (red) OGNVs located in liver Kupffer cells (F4/80⁺, green) were visualized with confocal microscopy at 1 d and 5 d after administer of clodronate. Data are representative of three independent experiments. (FIG. 19D) Representative for the treatment effect on liver metastasis (left, upper panel) and hematoxylin and eosin (H&E)-stained liver sections (left bottom panel) from Kupffer cell depleted mice with or without RAW264.7 cells adoptively transferred, Right; Liver weight. (FIG. 19E) Frequency of IFNγpositive cells (left panel) in liver CD3⁺Dx5⁺ (NKT) cells, CD3⁻Dx5⁺ (NK) cells, and CD3⁺Dx5⁻ (T) cells from OGNVs/Ctrl miRNA and OGNVs/miR18a treated mice with/without macrophages pre-depleted. The percentages of positive NK, NKT, and T cells are shown (right panel); each symbol represents an individual mouse. *P<0.05 (two-tailed t-test). Data are representative of three independent experiments (error bars, S.E.M).

FIGS. 20A-20H include graphs and images illustrating that miR-18a mediated inhibition of the growth of liver metastasis of colon tumor cells is IFNγ dependent. (FIG. 20A) Representative livers (up) (metastatic nodules shown by arrows) and H&E-stained sections of livers (middle, 20×; bottom, 400× magnification) from IFNγ knockout (KO) naïve mice. Liver weight of IFNγ KO mice (bottom). (FIG. 20B) Frequency of IFNγ⁺F4/80⁺ cells in liver from IFNγ KO mice (Naive) and CT26 liver metastatic mice was assessed by flow cytometry. The percentages of IFNγ⁺F4/80⁺ cells in liver and each symbol represents the FACS data from individual mice (right panel). (FIG. 20C) Frequency of IL-12, TGFIβ, MHCII positive cells in liver F4/80⁺ cells from IFNγ KO mice was assessed by flow cytometry. The percentages of double positively stained cells from treated mice are presented, and each symbol represents the FACS data from individual mice (right panel). (FIG. 20D) Representative livers (upper) and H&E-stained sections of livers (middle, 20×; bottom, 400× magnification) from NOG mice treated as labeled in the figure are shown (upper panel), and liver weight of NOG mice treated as labeled in the figure is indicated (bottom panel). (FIG. 20E) Frequency of liver F4/80⁺IFNγ⁺, F4/80⁺IL-12⁺, F4/80⁺MHCII⁺ and F4/80⁺TGFβ⁺ cells from NOG mice treated as indicated in the labels of FIG. 20E. Percent double positive cells (right panels). (FIG. 20F) Representative livers (up) from athymic nude mice. Middle: liver weight. Bottom: quantification of liver metastatic foci. (FIG. 20G) Frequency of IFNγ and IL-12 positive cells in liver F4/80⁺ KC cells. (FIG. 2011) Frequency of IFNγ positive cells in liver Dx5⁺NK cells. *P<0.05 (two-tailed t-test). Data are representative of three independent experiments (error bars, S.E.M.).

FIGS. 21A-B include graphs illustrating the frequency of CD3⁺ and Dx5⁺ cells in naïve and tumor bearing NOG mice. (FIG. 21A) Graphs illustrating F4/80⁺ cells in in naïve and tumor bearing NOG mice. (FIG. 21B) Graph illustrating the frequency of CD3⁺ and Dx5⁺ cells in naïve and tumor bearing NOG mice.

FIGS. 22A-22H include graphs and images illustrating that miR-18a suppresses liver metastasis of colon cancer triggered by direct targeting of Irf2 expressed in Kupffer cells. (FIG. 22A) Schematic diagram of the putative binding sites of miR-18a in the wide type (WT) IRF2 3′ untranslated regions (UTR). The miR-18a seed matches in the IRF2 3′UTR are mutated at the positions as indicated. CDS, coding sequence. (FIG. 22B) Expression of miR-18a and potential miR-18a targeted genes in macrophages-like RAW264.7 cells was analyzed by real-time PCR. (FIG. 22C) Expression of candidate miRN-18a target gene IRF2 and IFNγ in macrophage RAW264.7 cells assessed by western blotting. (FIG. 22D) IRF2 (red) expression in liver of CT26/OGNVs and CT26/OGNVs/miR-18a treated mice, visualized with a confocal microscopy. Data are representative of three independent experiments (n=5). (FIG. 22E) Evaluation of IRF2 and IFNγ level in macrophage-like RAW264.7 cells assessed by qPCR, 72 h after transfection of IRF2 siRNA (si-IRF2) or control (Ctrl) siRNA. (FIG. 22F) Expression of IRF2 and IFNγ in aliquots of macrophage-like RAW264.7 cells assessed by western blotting (left), quantification of results (right). (FIG. 22G) Expression of miR-18a and candidate miR-18a target genes in liver F4/80⁺ cells sorted by FACS and assessed by real-time PCR, following intravenous administration of OGNVs/miR-18a mimic and OGNVs/control miRNA. (FIG. 22H) Luciferase activity assays of WT and mutated Irf2 3′UTR luciferase reporters after co-transfection with miR-18a mimic, miRNA mimic control, miR-18a anti-sense RNA (AS-miR-18a), or miRNA anti-sense negative control RNA in RAW264.7 cells. The luciferase activity of each sample was normalized to the Renilla luciferase activity. The normalized luciferase activity of transfected control mimic miRNA was set as relative luciferase activity of 1. Error bars represent S.E.M. Each data point was measured in triplicate.

FIG. 23 includes images illustrating up-regulation of IRF2 in metastatic liver tissue of colon cancer patients. Double staining of human colon cancer tissue sections with antibodies against IRF2 (green) and against CD68 (red) followed by detection of fluorescence.

FIG. 24 includes a schematic of proposed pathways leading to induction of M1 macrophages mediated by miR-18a. miR-18a encapsulated by OGNVs (OGNVs/miR18a) is taken up by liver macrophages, leading to down-regulation of IRF2. As a result of decreased IRF2, IFNγ is upregulated and subsequently stimulates the induction of M1 macrophages (F4/80⁺IL-12⁺) which further triggers anti-tumor activation of NK, NKT, and T cells. a

### BRIEF DESCRIPTION OF THE SEQUENCE LISTING

SEQ ID NO: 1 is a nucleic acid sequence of a forward mm-TGFβ primer for quantitative Real-Time PCR (qPCR) of mRNA;

SEQ ID NO: 2 is a nucleic acid sequence of a reverse mm-TGFβ primer for quantitative Real-Time PCR (qPCR) of mRNA;

SEQ ID NO: 3 is a nucleic acid sequence of a forward mm-IFNγ primer for quantitative Real-Time PCR (qPCR) of mRNA;

SEQ ID NO: 4 is a nucleic acid sequence of a reverse mm-IFNγ primer for quantitative Real-Time PCR (qPCR) of mRNA;

SEQ ID NO: 5 is a nucleic acid sequence of a forward mm-MHCII primer for quantitative Real-Time PCR (qPCR) of mRNA;

SEQ ID NO: 6 is a nucleic acid sequence of a reverse mm-MHCII primer for quantitative Real-Time PCR (qPCR) of mRNA;

SEQ ID NO: 7 is a nucleic acid sequence of a forward mm-IL-12 primer for quantitative Real-Time PCR (qPCR) of mRNA;

SEQ ID NO: 8 is a nucleic acid sequence of a reverse mm-IL-12 primer for quantitative Real-Time PCR (qPCR) of mRNA;

SEQ ID NO: 9 is a nucleic acid sequence of a forward mm-SMAD2 primer for quantitative Real-Time PCR (qPCR) of mRNA;

SEQ ID NO: 10 is a nucleic acid sequence of a reverse mm-SMAD2 primer for quantitative Real-Time PCR (qPCR) of mRNA;

SEQ ID NO: 11 is a nucleic acid sequence of a forward mm-ESR1 primer for quantitative Real-Time PCR (qPCR) of mRNA;

SEQ ID NO: 12 is a nucleic acid sequence of a reverse mm-ESR1 primer for quantitative Real-Time PCR (qPCR) of mRNA;

SEQ ID NO: 13 is a nucleic acid sequence of a forward mm-ESR2 primer for quantitative Real-Time PCR (qPCR) of mRNA;

SEQ ID NO: 14 is a nucleic acid sequence of a reverse mm-ESR2 primer for quantitative Real-Time PCR (qPCR) of mRNA;

SEQ ID NO: 15 is a nucleic acid sequence of a forward mm-IRF1 primer for quantitative Real-Time PCR (qPCR) of mRNA;

SEQ ID NO: 16 is a nucleic acid sequence of a reverse mm-IRF1 primer for quantitative Real-Time PCR (qPCR) of mRNA;

SEQ ID NO: 17 is a nucleic acid sequence of a forward mm-IRF2 primer for quantitative Real-Time PCR (qPCR) of mRNA;

SEQ ID NO: 18 is a nucleic acid sequence of a reverse mm-IRF2 primer for quantitative Real-Time PCR (qPCR) of mRNA;

SEQ ID NO: 19 is a nucleic acid sequence of a forward mm-LEF primer for quantitative Real-Time PCR (qPCR) of mRNA;

SEQ ID NO: 20 is a nucleic acid sequence of a reverse mm-LEF primer for quantitative Real-Time PCR (qPCR) of mRNA;

SEQ ID NO: 21 is a nucleic acid sequence of a forward mm-TCF primer for quantitative Real-Time PCR (qPCR) of mRNA;

SEQ ID NO: 22 is a nucleic acid sequence of a reverse mm-TCF primer for quantitative Real-Time PCR (qPCR) of mRNA;

SEQ ID NO: 23 is a nucleic acid sequence of a forward mm-AXIN2 primer for quantitative Real-Time PCR (qPCR) of mRNA;

SEQ ID NO: 24 is a nucleic acid sequence of a reverse mm-AXIN2 primer for quantitative Real-Time PCR (qPCR) of mRNA;

SEQ ID NO: 25 is a nucleic acid sequence of a forward mm-Wnt7a primer for quantitative Real-Time PCR (qPCR) of mRNA;

SEQ ID NO: 26 is a nucleic acid sequence of a reverse mm-Wnt7a primer for quantitative Real-Time PCR (qPCR) of mRNA;

SEQ ID NO: 27 is a nucleic acid sequence of a forward primer for mutantgenesis;

SEQ ID NO: 28 is a nucleic acid sequence of a reverse primer for mutantgenesis;

SEQ ID NO: 29 is a nucleic acid sequence of a forward primer for sequencing of a mutant; and

SEQ ID NO: 30 is a nucleic acid sequence of a reverse primer for sequencing of a mutant.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Some of the polynucleotide and polypeptide sequences disclosed herein are cross-referenced to GENBANK®/GENPEPT® accession numbers. The sequences cross-referenced in the GENBANK®/GENPEPT® database are expressly incorporated by reference as are equivalent and related sequences present in GENBANK®/GENPEPT® or other public databases. Also expressly incorporated herein by reference are all annotations present in the GENBANK®/GENPEPT® database associated with the sequences disclosed herein. Unless otherwise indicated or apparent, the references to the GENBANK®/GENPEPT® database are references to the most recent version of the database as of the filing date of this Application.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Microvesicles are naturally existing nanoparticles that are in the form of small assemblies of lipid particles, are about 50 to 1000 nm in size, and are not only secreted by many types of in vitro cell cultures and in vivo cells, but are commonly found in vivo in body fluids, such as blood, urine and malignant ascites. Indeed, microvesicles include, but are not limited to, particles such as exosomes, epididimosomes, argosomes, exosome-like vesicles, microparticles, promininosomes, prostasomes, dexosomes, texosomes, dex, tex, archeosomes, and oncosomes.

As noted above, microvesicles can be formed by a variety of processes, including the release of apoptotic bodies, the budding of microvesicles directly from the cytoplasmic membrane of a cell, and exocytosis from multivesicular bodies. For example, exosomes are commonly formed by their secretion from the endosomal membrane compartments of cells as a consequence of the fusion of multivesicular bodies with the plasma membrane. The multivesicular bodies are formed by inward budding from the endosomal membrane and subsequent pinching off of small vesicles into the luminal space. The internal vesicles present in the MVBs are then released into the extracellular fluid as so-called exosomes.

As part of the formation and release of microvesicles, unwanted molecules are eliminated from cells. However, cytosolic and plasma membrane proteins are also incorporated during these processes into the microvesicles, resulting in microvesicles having particle size properties, lipid bilayer functional properties, and other unique functional properties that allow the microvesicles to potentially function as effective nanoparticle carriers of therapeutic agents. In this regard, the term “microvesicle” is used interchangeably herein with the terms “nanoparticle,” “liposome,” “exosome,” “exosome-like particle,” “nanovesicle,” “nano-vector” and grammatical variations of each of the foregoing.

With further respect to microvesicles, the presently-disclosed subject matter is based, at least in part, on the discovery that edible plants, such as fruits, are not only a viable source of large quantities of microvesicles, but that microvesicles derived from edible plants can be used as an effective delivery vehicle for miRNA, while also retaining the biological activity of the miRNA.

The presently-disclosed subject matter thus includes edible plant-derived microvesicle compositions that further include miRNA and are useful in the treatment of various diseases, including cancers. In some embodiments of the presently-disclosed subject matter, a microvesicle composition is provided that comprises an miRNA encapsulated by an microvesicle, wherein the microvesicle is derived from an edible plant. In some embodiments, the miRNA encapsulated by the edible-plant derived microvesicle is selected from miR18a and miR17.

The term “edible plant” is used herein to describe organisms from the kingdom Plantae that are capable of producing their own food, at least in part, from inorganic matter through photosynthesis, and that are fit for consumption by a subject, as defined herein below. Such edible plants include, but are not limited to, vegetables, fruits, nuts, and the like. In some embodiments of the microvesicle compositions described herein, the edible plant is a fruit. In some embodiments, the fruit is selected from a grape, a grapefruit, and a tomato.

The phrase “derived from an edible plant,” when used in the context of a microvesicle derived from an edible plant, refers to a microvesicle that, by the hand of man, exists apart from its native environment and is therefore not a product of nature. In this regard, in some embodiments, the phrase “derived from an edible plant” can be used interchangeably with the phrase “isolated from an edible plant” to describe a microvesicle of the presently-disclosed subject matter that is useful for encapsulating therapeutic agents.

The phrase “encapsulated by a microvesicle,” or grammatical variations thereof is used herein to refer to microvesicles whose lipid bilayer surrounds a therapeutic agent. For example, a reference to “microvesicle miRNA” refers to an microvesicle whose lipid bilayer encapsulates or surrounds an effective amount of miRNA. In some embodiments, the encapsulation of various therapeutic agents within microvesicles can be achieved by first mixing the one or more of the miRNA with isolated microvesicles in a suitable salt solution, such as a 155 mM NaCl solution. After a period of incubation, the microvesicle/miRNA agent mixture is then subjected to a sucrose gradient (e.g., and 8, 30, 45, and 60% sucrose gradient) to separate the UV radiation, sonication, and a centrifugation step to isolate the microvesicles encapsulating the therapeutic agents. After this centrifugation step, the microvesicles including the miRNA can then be collected, washed, and dissolved in a suitable solution for use as described herein below.

MicroRNAs are naturally occurring, small non-coding RNAs that are about 17 to about 25 nucleotide bases (nt) in length in their biologically active form. miRNAs post-transcriptionally regulate gene expression by repressing target mRNA translation. It is thought that miRNAs function as negative regulators, i.e. greater amounts of a specific miRNA will correlate with lower levels of target gene expression. There are three forms of miRNAs existing in vivo, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nt overhang at the 3′ end. The cleavage product, the premature miRNA (pre-miRNA) is about 60 to about 110 nt long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nt in length.

In some embodiments, the microvesicle compositions disclosed herein are transported to a subject's brain after administration to the subject. For example, in one embodiment, the microvesicle composition is transported to a subject's brain following intranasal administration. In another embodiment, following intranasal administration, the microvesicle composition is transported to the olfactory bulb, hippocampus, thalamus, and/or cerebellum. In contrast thereto, similarly administered DOTAP, a standard liposome, phosphate-buffered saline (PBS), and free DIR-dye were not transported to the brain. In a further embodiment, the microvesicle composition is transported to a subject's brain following oral administration. Other suitable routes of administration for transporting the microvesicle composition to the brain include any route capable of delivering the microvesicle composition to the subject.

In some embodiments, the microvesicle compositions disclosed herein facilitate delivery of RNA to the brain without or substantially without degradation of the RNA. Additionally or alternatively, the microvesicle composition may include a nanovector hyrided with polyethylenimine (PEI) (pNV). For example, in one embodiment, the pNV includes a grapefruit derived nanovector (GNV) hyrided with polyethylenimine (PEI) (pGNV). In some embodiments, the pNV and/or pGNV provide an increased capacity for carrying RNA as compared to NV and/or GNV. In some embodiments, the pNV and/or pGNV reduces or eliminates the toxicity induced by a PEI vector alone.

In some embodiments of the presently disclosed subject matter, a pharmaceutical composition is provided that comprises an edible plant-derived microvesicle composition disclosed herein and a pharmaceutical vehicle, carrier, or excipient. In some embodiments, the pharmaceutical composition is pharmaceutically-acceptable in humans. Also, as described further below, in some embodiments, the pharmaceutical composition can be formulated as a therapeutic composition for delivery to a subject.

A pharmaceutical composition as described herein preferably comprises a composition that includes pharmaceutical carrier such as aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutical compositions used can take such forms as suspensions, solutions or emulsions in oily or aqueous vehicles, and can contain formulatory agents such as suspending, stabilizing and/or dispersing agents. Additionally, the formulations can be presented in unit-dose or multi-dose containers, for example sealed ampoules and vials, and can be stored in a frozen or freeze-dried or room temperature (lyophilized) condition requiring only the addition of sterile liquid carrier immediately prior to use.

In some embodiments, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone, hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of capsules, tablets or lozenges formulated in conventional manner.

Various liquid and powder formulations can also be prepared by conventional methods for inhalation into the lungs of the subject to be treated or for intranasal administration into the nose and sinus cavities of a subject to be treated. For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the desired compound and a suitable powder base such as lactose or starch.

The compositions can also be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt).

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compositions can be administered by the drip method, whereby a formulation including a pharmaceutical composition of the presently-disclosed subject matter and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the composition can be prepared and administered as a suspension in an aqueous base or a pharmaceutically-acceptable oil base, such as an ester of a long chain fatty acid, (e.g., ethyl oleate).

In addition to the formulations described above, the microvesicle compositions of the presently-disclosed subject matter can also be formulated as rectal compositions, such as suppositories or retention enemas, e.g., containing conventional suppository bases such as cocoa butter or other glycerides. Further, the exosomal compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt.

Still further provided, in some embodiments, are methods for treating a cancer. In some embodiments, a method for treating a cancer is provided that comprises administering to a subject in need thereof an effective amount of an edible-plant derived microvesicle composition of the presently-disclosed subject matter (i.e., where a microvesicle encapsulates a miRNA). For example, in one embodiment, the microvesicle composition disclosed herein provides targeted delivery of an miRNA to tumor and/or cancer cells. In another embodiment, administration of the microvesicle composition disclosed herein inhibits tumor growth. As used herein, the term “cancer” refers to all types of cancer or neoplasm or malignant tumors found in animals, including leukemias, carcinomas, melanoma, and sarcomas.

By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some embodiments, the cancer is selected from the group consisting of colon cancer, brain cancer, and liver cancer. In some particular embodiments, the cancer is a liver metastases.

In some embodiments, the edible plant-derived microvesicle compositions used to treat the cancer further comprise a cancer targeting moiety or, in other words, a moiety that is capable of preferentially directing a composition of the presently-disclosed subject matter to a cancer cell. Such cancer targeting moieties include, but are not limited to, small molecules, proteins, or other agents that preferentially bind to cancer cells. For example, in some embodiments, the cancer targeting moiety can be an antibody that specifically binds to an epitope found predominantly or exclusively on a cancer cell. As another example, in some embodiments, the cancer targeting moiety is folic acid, as folic acid or folate receptors have been found to be overexpressed on a variety of different types of cancer.

For administration of a therapeutic composition as disclosed herein (e.g., an edible plant-derived microvesicle encapsulating a therapeutic agent), conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082).

Regardless of the route of administration, the compositions of the presently-disclosed subject matter are typically administered in amount effective to achieve the desired response. As such, the term “effective amount” is used herein to refer to an amount of the therapeutic composition (e.g., a microvesicle encapsulating a miRNA and a pharmaceutically vehicle, carrier, or excipient) sufficient to produce a measurable biological response (e.g., a decrease in inflammation). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

### EXAMPLES

Materials and Methods for Examples 1-4.

Reagents. DOTAP/DOPE mixture (790310C) was purchased from Avanti Polar Lipids, Inc. The Dual-Luciferase Report Assay System was purchased from Promega. Luciferase GL3 Duplex was purchased (Dharmacon). miR-17 mimics (Sequence: CAAAGUGCUUACAGUGCAGGUAG, Catalog number: 4464066, Life Technologies) and Dylight547 labeled miR17 (Sequence: UGGAAGACUAGUGAUUUUGUUGU-DY547) was synthesized by Life Technologies. Near IR fluorescein dye DiIC18(7) (1,1′-Dioctadecyl-3,3,3′,3′-Tetramethylindotricarbocyanine Iodide) (DiR) and SYTO® 60 Red Fluorescent dye (Syto60) was purchased from Life Technologies. Polyethylenimine, branched (average MW-25000, Cat#: 408727), folic acid, glutaraldehyde, cacodylate buffer, sucrose and paraformaldehyde were purchased from Sigma.

Antibodies. The following antibodies were used: rabbit anti-Ibal antibody that specifically recognizes microglial cells and macrophages (Wako Chemicals, Richmond, Va.), anti-folate receptor(N-20) (Santa Cruz Biotechnology), anti-luciferase (Santa Cruz Biotechnology), anti-F4/80 (BM8, eBioscience), anti-mouse MHC Class I (eBioscience), anti-mouse CD49b (DX5) (eBioscience), IRDye® 800CW goat anti-mouse IgG (H+L) (LI-COR Biosciences). The following secondary antibodies were purchased from Life Technologies: Alexa fluor 594 conjugated goat anti-rat IgG (H+L) (A11007), Alexa fluor 488 conjugated rabbit anti-mouse IgG (H+L) (A11059), Alexa fluor 488 conjugated chicken anti-goat IgG (H+L) (A21467), Alexa fluor 680 conjugated goat anti-rabbit IgG (H+L) (A21109), and Alexa fluor 488 conjugated goat anti-rabbit IgG (H+L) (A11008).

Cell line. The mouse (H-2b) glioblastoma cell line GL26 stably expressing the luciferase gene (GL26-Luc) was provided by Dr. Behnam Badie (Beckman Research Institute of the City of Hope, Los Angeles, Calif.), and maintained in RPMI-1640 media supplemented with 10% heat-inactivated fetal bovine serum in a humidified CO2 incubator at 37° C.

Animals. C57BL/6j mice (H-2b) were purchased from the Jackson Laboratory (Bar Harbor, Me.). Animals were housed in the animal facility at the University of Louisville per an Institutional Care and Use Committee-approved protocol.

Preparation of grapefruit-derived nanovectors GNVs, pGNVs, and FA-pGNVs. All GNVs used in this study were prepared according to a previously described protocol[6]. pGNVs were made of PEI/RNA and GNV complex. PEI/RNA complex was formed by adding PEI in PBS to RNA extracted from EL4 cells, synthetized miR17, or Dylight 547 labeled miR17 (miR17-Dy547) or siRNA-luc or scramble siRNA (PEI/RNA=10:1, in weight) and the mixtures were then incubated at 25° C. for 30 min for formation of PEI/RNA complex. The PEI/RNA complex was added to the film of lipids extracted from grapefruit nanoparticles using a described method[6]. Samples were sonicated in a bath-sonicator (F560 bath sonicator, Fisher Scientific, Pittsburg, Pa.) for 15 min, and sonication repeated 3 times, and followed by ultracentrifuge at 100,000× g for 90min at 4 ° C. to wash unbound RNA or PEI/RNA from the PEI/RNA/GNV complexes. The efficiency of RNA associated with pGNVs was demonstrated by measuring the amount of RNase pre-digested miR17-Dy547 encapsulated in pGNVs (pGNV/miR17-Dy547) using a fluorescence microplate reader (EX/Em=530/590 nm). The amount of miR17 carried by the pGNVs was calibrated based on a comparison to a standard curve generated from synthesized miR17-Dy547 of known concentrations and expressed as ng of Dy547-miR17/1mM of GNVs. The efficiency of miR17 carried by pGNVs was expressed as %=amounts of miR17-Dy547 carried by pGNVs/ total amounts of miR17-Dy547 initially added to PEI or GNVs ×100. Before being used in experiments the pGNVs were homogenized by passing them through a high pressure homogenizer (Avestin Inc., Ottawa, Canada) using a protocol provided in the homogenizer instruction manual. For production of FA-pGNVs, total lipids was extracted from sucrose purified grapefruit nanoparticles by the Bligh and Dyer method[28] and quantified using the phospholipid assay of Rouser. Folic acid (12.5 μg in DMSO) was added to the lipid (1mM phospholipid in chloroform) extracted from grapefruit nanoparticles and a film was formed by being dried under nitrogen gas before adding the PEI-RNA complex to make FA-pGNVs using an identical protocol as described for making pGNVs. The density of sucrose-banded GNV, GNV/RNA, and pGNV/RNA was determined by measuring the refractive index of a 10-μL aliquot with an Abbe refractometer (Leica Mark II plus) at a constant temperature of 20° C. The PEI associated with PEI/RNA and pGNVs was quantitatively analyzed with a method as described.

Intranasal delivery of GNVs, pGNVs, and FA-pGNVs in mice. For intranasal administration of GNVs, pGNVs, and FA-pGNVs, C57BL/6j mice were anesthetized by I.P. injection of a ketamine/xylazine mixture (40 mg/5 mg/kg body weight) and each mouse placed in a supine position in an anesthesia chamber. PBS (2 μl) containing GNVs, pGNVs, or FA-pGNVs (20 nmol/2 μ) was administered intranasally as drops with a small pipette every 2 minutes into alternating sides of the nasal cavity for a total of 20 minutes. A total volume of 20 μl was delivered into the nasal cavity.

Evaluation of brain inflammation. Mice were administered intranasally with pGNVs or PEI-RNA complex (3.0 μg is RNA/mouse) using the method described above. Bacterial LPS (2.5 mg/kg; Sigma-Aldrich) was injected intraperitoneally into C57BL/6j mice as a control for induction of brain inflammation. After intranasal administration, mice were transcardially perfused with PBS followed by a 4% paraformaldehyde solution at pH 7.4. Brain tissue was post-fixed overnight in 4% paraformaldehyde and then cryopreserved in phosphate-buffered 30% sucrose. Brains were embedded in OCT compound (Tissue-Tek; Sakura, Torrance, Calif.) and kept at −20 ° C. overnight. Brain tissue sections were cut with a cryostat (30-μm thick) and the tissue sections stored at −20° C. Immunofluorescent staining of microglial cells with rabbit anti-Ibal antibody or F4/80 antibody was carried out according to previously described procedures. Tissues evaluated for the presence of Iba1 or F4/80 positive staining were assessed using a Zeiss LSM 510 confocal microscope equipped with a digital image analysis system (Pixera, San Diego, Calif.).

Ex vivo imaging. To monitor the trafficking of GNVs administered intranasally, GNVs were first labeled using a near-infrared lipophilic carbocyanine dye-dioctadecyl-tetramethylindotricarbocyanine iodide (DIR, Invitrogen, Carlsbad, Calif.) using a previously described method. To localize GNVs in brain tissue, the DIR-labeled GNVs (10 μg/10 μl in PBS) were administered intranasally to C57BL/6j mice as described above in the method section of this study. The brains of treated mice were imaged over a 24-hour period using a prototype LI-COR imager (LI-COR Biosciences). For controls, mice (five per group) received either DOTAP liposomes or nonlabeled GNVs in PBS or free DIR dye at the same concentration for DIR dye-labeled GNVs.

Brain tumor-bearing mice model. 2×104 GL26-Luc cells were intracranially injected per mouse using a method described previously. In brief, 2 μL of PBS containing 5×104 tumor cells were injected at the coronal suture, 1 mm lateral to the midline, and 3 mm deep into the frontal lobes, using a Hamilton syringe (Fisher Scientific). Tumor-bearing mice were treated every three days for 21 days beginning on day 5 after the tumor cells were implanted at a dose of 20 μg of miR17 or miRNA scramble carried by FA-pGNVs or FA-pGNVs in PBS as a control. All mice were monitored every day and euthanized when they exhibited neurological symptoms indicative of impending death. Monitoring the growth of injected tumor cells was accomplished by quantifying luciferase activity over a 28-day period at 5-days post-tumor cell injection using a previously described method. For evaluating the tumor targeted delivery efficiency of FA-pGNVs, siRNA luciferase was carried by FA-pGNVs (FA-pGNV/siRNA luciferase) and 15-day tumor bearing mice were intranasally administrated FA-pGNV/siRNA luciferase or FA-pGNV/scramble siRNA as a control and luciferase activity of brain tumor bearing mice was analyzed. Regions of interest were analyzed for luciferase signals using Living Image 2.50 software (Xenogen) and were reported in units of relative photon counts per second. The total photon count per minute (photons/minute) was calculated (five animals) using Living Image software. The effects of treatment versus non-treatment on brain tumor-bearing mice was determined by dividing the number of photons collected for treated mice at different imaging time points by the number of photons collected at zero imaging time. Results were represented as pseudocolor images indicating light intensity. The effects of treatment versus non-treatment on brain tumor-bearing mice on the induction of DX5 NK cells in the GL-26-luc tumor was also evaluated by immune-staining of post-fixed brain tissue with anti- DX5, luciferase and MHCI antibodies according to previously described procedures.

Size and surface charge of GNV, pGNVs, and FA-pGNVs were determined by Zetasizer Nano S90.

EM analysis. GNVs were fixed with 2% glutaraldehyde in 0.1 M cacodylate buffer (pH 7.4) for 4 h, at 4° C. After an extensive wash in the same buffer, samples were removed, post-fixed for 1 h at 22° C. with 1% osmium tetroxide in 0.1 M cacodylate buffer (pH 7.4) and coated with gold-palladium, and observed with a Zeiss Supra 35 VP at an accelerating voltage of 10 kV.

Cellular FA-pGNVs uptake experiments. The folate mediated targeting efficiency of FA-pGNVs was determined by in vitro incubation of GL-26-luc cells with miR17-Dy547-loaded or Syto60 labeled RNA-loaded folate-pGNVs. Briefly, after removing the culture medium, cells were washed once with PBS and RPMI 1640 (200 μl) then added to each well. FA-pGNV/miR17-Dy547, pGNV/miR17-Dy547, FA-pGNV/Syto60-RNA, or pGNV/Syto60-RNA (10 nmol/μl) was added to each well. Cells were then incubated for variable time points at 37° C. in a 5% CO2 incubator. Following incubation, cells were placed on ice, washed three times with 100 μl of ice-cold PBS to remove extracellular FA-pGNVs/miR17-Dy54,7 pGNVs/miR17-Dy547, FA-pGNVs/Syto60-RNA, or pGNVs/Syto60-RNA. The fluorescence intensity of the cells was measured using a fluorescence spectrometer (Synergy HT, BioTek) at an excitation/emission of 530 nm/590 nm or Dylight547 labeled miR17+ cells (red) or Syto60 labeled RNA were assessed with Zeiss LSM 510 confocal microscope equipped with a digital image analysis system (Pixera, San Diego, Calif.).

The amount of miR17 in the transfected GL-26 cells was quantitatively analyzed with qPCR using a described method. The specificity of the folate-targeted FA-pGNV/miR17-Dy547 for GL-26-luc cells expressing the folate receptor was determined by performing cellular competitive binding experiments. In these experiments, FA-pGNVs/miR17-Dy547 (10 nmol/μl) was premixed with variable amount of free folate and then mixed samples were added to a 12 h culture of GL-26-luc cells. miR17-Dy547 concentrations in cells were determined by measuring Dylight547 from microplate reader at Ex/Em=530 nm/590 nm.

Flow Cytometry. GL-26-luc cell lines were digested and centrifuged at 800× g and cell pellets were resuspended in FACS buffer (PBS, 1% BSA, 0.1% EDTA). Cells were pretreated on ice with the FcγR-blocking mAb (eBioscience) for 10 minutes. This step was followed by treating with anti-mouse MHC class I (eBioscience) for 30 minutes on ice. All data were analyzed using FlowJo FACS software.

### Example 1

### Intranasally Administered GNVs are Transported to the Brains of Mice

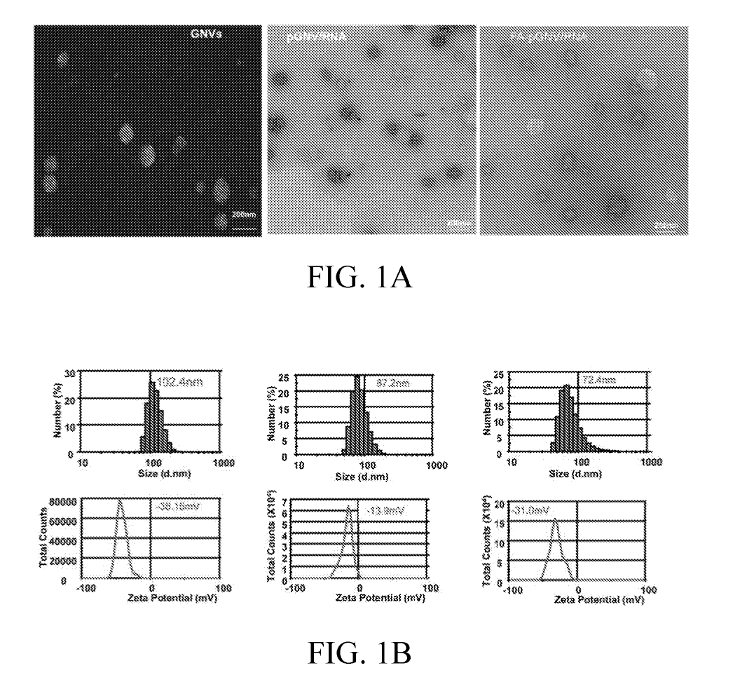

Using standard techniques, we isolated edible plant exosome-like nanoparticles from the juice of grapefruit, and nanoparticles were made with lipids extracted from grapefruit exosome-like nanoparticles. The nanoparticles were fully characterized based on electron microscopic examination (FIG. 1A) of a sucrose gradient purified band, charge, and size distribution (FIG. 1B).

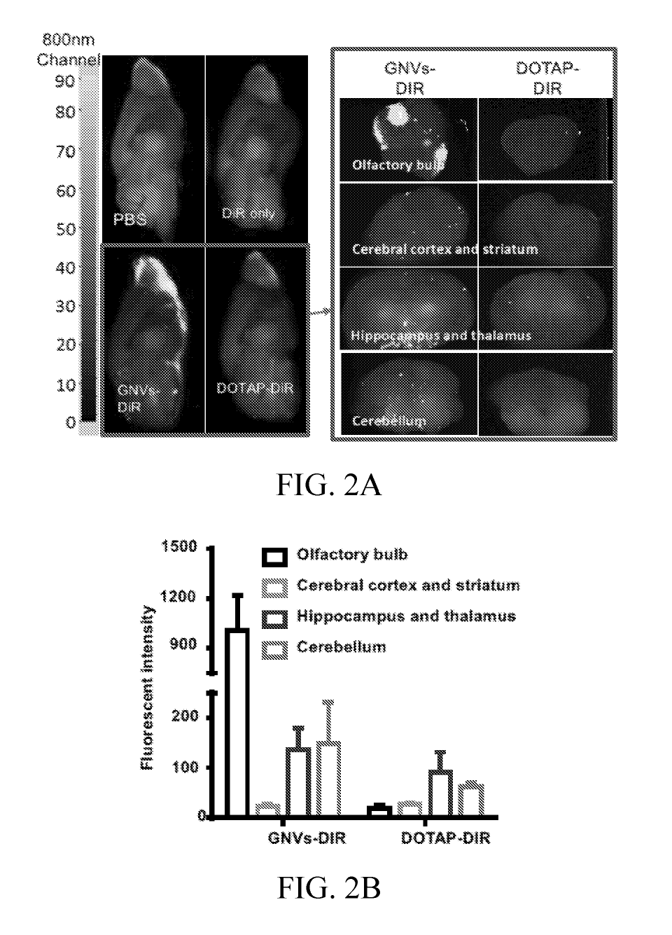

To determine whether GNVs can be transported intranasally into the brain, DIR-dye-labeled GNVs were administered using a small pipette as ten 2-μl doses in alternating sides of the nose spaced 2 minutes apart. 12 h after intranasal delivery, mouse brains were examined for the presence of the GNVs using an Odyssey scanner. DIR fluorescent labeled GNVs were observed in the brain with their primary location being in the olfactory bulb, hippocampus, thalamus and Cerebellum, suggesting that translocation of GNVs to the brain occurred within a short time (FIG. 2). In contrast, a standard liposome, DOTAP, commonly used for gene transfer, was not detected in the brain (FIG. 2). Very little or no fluorescence was detected in the brain of mice intranasally administered phosphate-buffered saline (PBS) or free DIR-dye (FIG. 2). These results suggest that GNVs have a unique property allowing for intranasal transfer or delivery to the brain. No apparent toxicity or behavioral abnormalities such as diarrhea, altered gait or skin inflammation, swelling, ulceration of the body or motor paralysis were observed in any of the mice during and after (21 days) the experiment.

### Example 2

### RNA Carried by GNVs is Intranasally Delivered to Brain

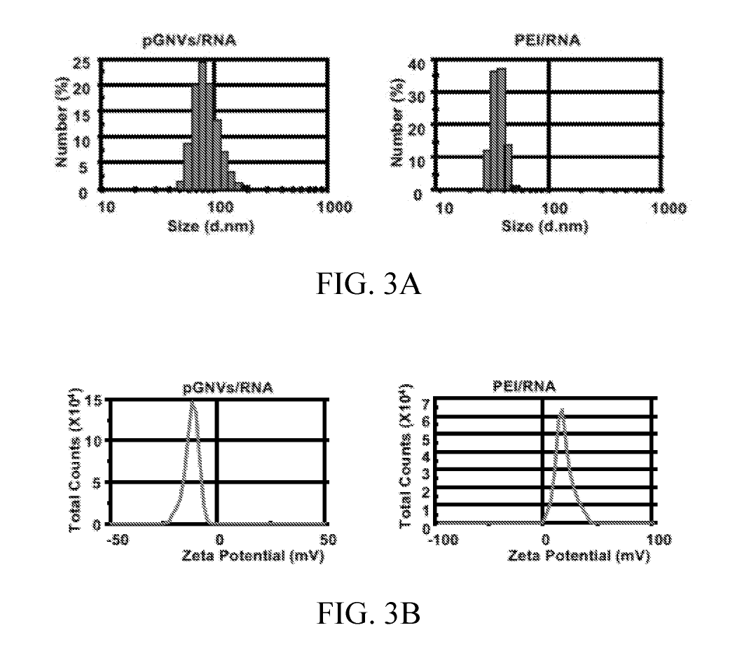

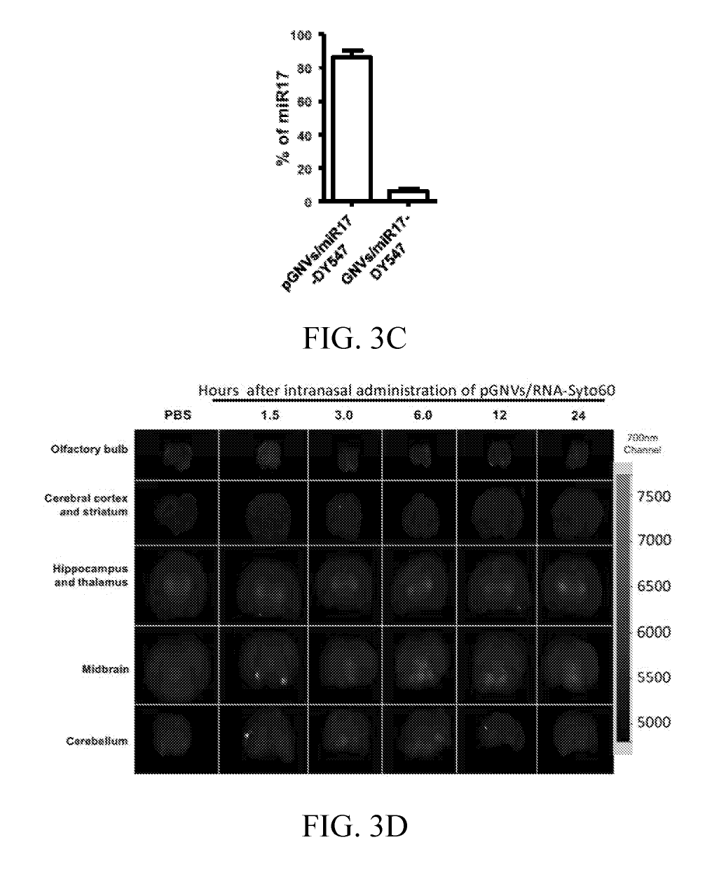

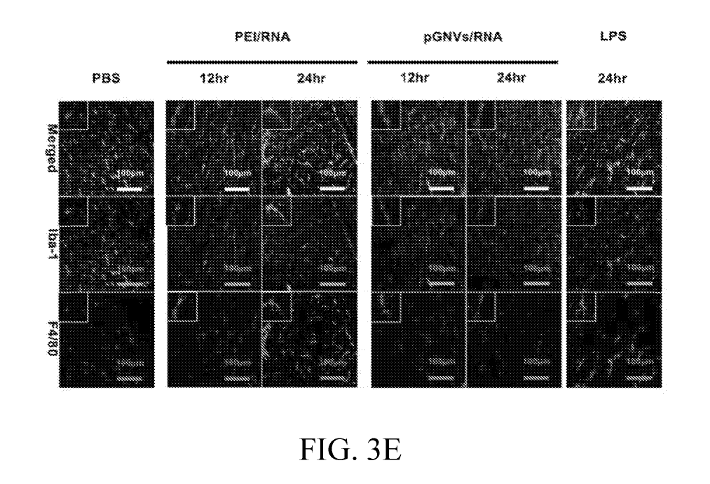

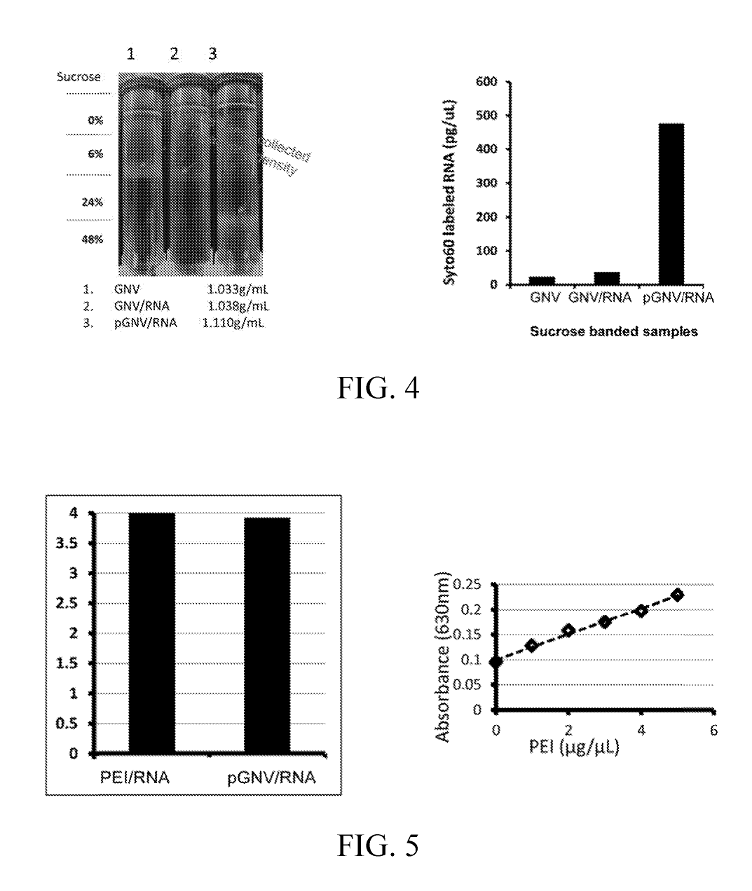

Given that intranasally administered GNVs are transported to the brains of mice, and delivering RNA through an intranasal route would have numerous applications for gene therapy of brain related disease, we next tested whether RNA carried by GNVs can be delivered without degradation to the brain. First, we tested whether the efficiency of GNVs for delivering RNA in general can be increased using PEI due to the reported higher efficiency of PEI in carrying RNA and DNA[7]. Increasing the capacity of RNA or DNA being encapsulated for potential intranasal delivery is an important factor because one of the limiting factors in the intranasal delivery is the amount of therapeutic reagents successfully delivered. To test this concept total RNAs were extracted from EL4 cells. PEI and cellular RNA were mixed (PEI/RNA) and subsequently added to lipid film extracted from grapefruit exosome-like nanoparticles and followed by sonication. The results showed that the PEI/RNA reassembled into GNVs (pGNV/RNA) with a diameter of 87.2±11.3 nm (means±standard error of the mean (s.e.m.); whereas, PEI/RNA has a diameter of 35.6±8.7 nm (FIG. 3A). Data presented in FIG. 3A (top panel) are supported by 1) EM examination showing that the PEI/RNA complex is smaller than the pGNV/RNA in size (FIG. 3A, bottom panel); 2) the location of pGNV/RNA after sucrose gradient centrifugation where it migrated to a different density than that of GNV/RNA which do not have PEI (FIGS. 4); and 3) pGNV/RNA has a higher sucrose density than GNVs (FIG. 4, 1.11 versus 1.03). Zeta potential values for the PEI/RNA complex were positive; whereas, pGNVs were negative. Values were in the range of 20.9 mV for the PEI/RNA complex and −13.9 mV for the pGNV/RNA complexes (FIG. 3B). Remarkably, the results generated from quantitative analysis of RNA extracted from pGNV/RNA and GNV/RNA indicate that the capacity of pGNVs to carry RNA is much higher (86.2±5.7%) than the GNVs (5.9±1.6%) (FIG. 3C). Next, we tested whether the RNA carried by pGNVs can be delivered to the brain through an intranasal route. Total RNAs extracted from the EL4 cell line were labeled with the fluorescent dye Syto60 for tracking RNA delivered by pGNVs. The imaging results from frozen sectioned brain indicated that a positive fluorescent signal was detected as early as 1.5 h after intranasal administration (FIG. 3D). The Syto60 labelled RNA signal was detected primarily in the olfactory bulb, midbrain and thalamus 12 h after intranasal administration. The size and charge of nanoparticles has an effect on their distribution in vivo. The fact that pGNVs are smaller in size than GNVs (FIGS. 1A-B) prompted us to further determine whether physiological distribution of pGNVs is different from that of GNVs after intranasal administration. The imaging results from frozen sectioned brain indicated that a stronger fluorescent signal was detected in the thalamus and midbrain of mice given pGNV/RNA than of mice given GNV/RNA. This result agrees with a reduction of DiR signal 12 h post-administration that is detected in the olfactory bulb of mice given pGNV/RNA (FIG. 3E).

PEI and nucleic acid complexes are toxic and directly linked to the positive charge on the surface of the complex. Next, we tested whether PEI/RNA complexed with GNVs is less toxic than PEI/RNA. Immune histological staining indicates that intranasal administration of PEI/RNA induces a large number of F4/80+ macrophages and Iba-1+ microglia cells whereas no induction was observed in the brain of mice intranasally administrated with pGNV/RNA in comparison with mice given PBS as a control (FIG. 3F). A lack of induction of F4/80+ macrophages and Iba-1+ microglia cells is most likely not due to a reduced amount of PEI in PEI/RNA when compared to pGNV/RNA since there was approximately the same amount of PEI in the PEI/RNA and pGNV/RNA (FIG. 5) detected. Collectively, combination of PEI and GNVs enhances the delivering RNA efficiency in GNVs and eliminates the toxicity induced by PEI vector.

### Example 3

### Intranasal Targeted Delivery of miR17 to Brain Tumor with FA-pGNVs

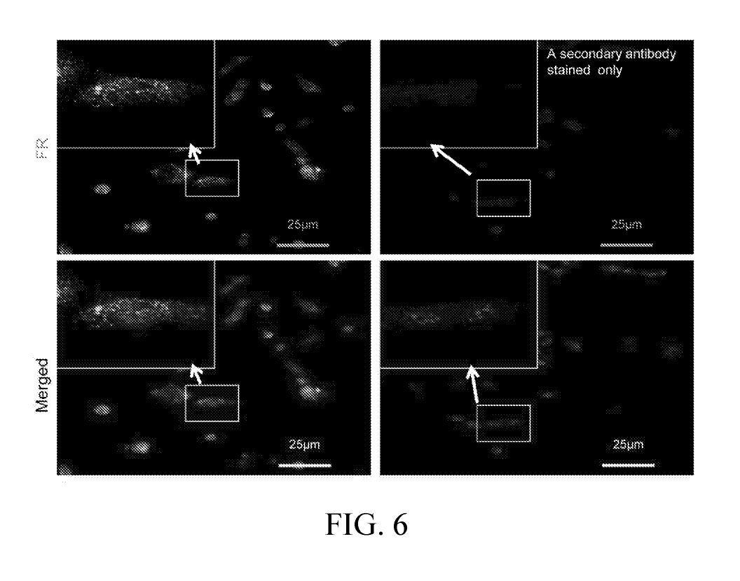

Since no adverse side-effects had been observed with an intranasal administration of pGNVs, we next tested whether pGNVs can be used as a therapeutic miRNA delivery vehicle. In cancer therapy, accurate targeting to tumor tissue is required for successful therapy. Therefore, we first tested whether pGNVs can be modified to achieve tumor targeting. High-affinity folate receptors (FRs) are expressed at elevated levels on many human tumors and in almost negligible amounts on non-tumor cells. Therefore, we tested whether pGNVs binding folic acid (FA) (FA-pGNVs) would significantly enhance pGNV targeting to GL-26 tumor cells which express folate receptors (FIG. 6).

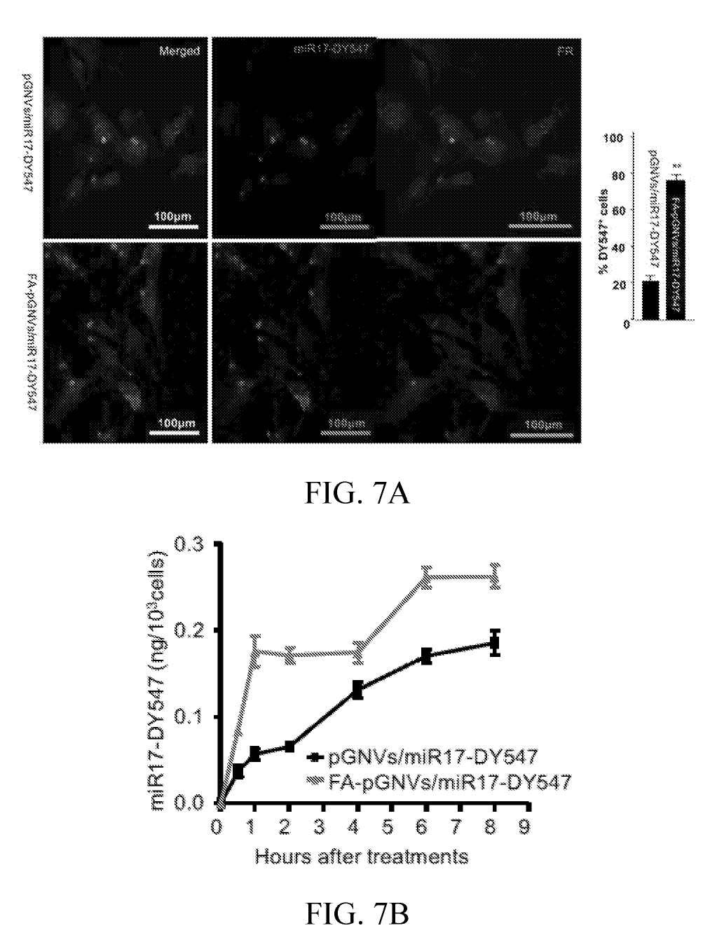

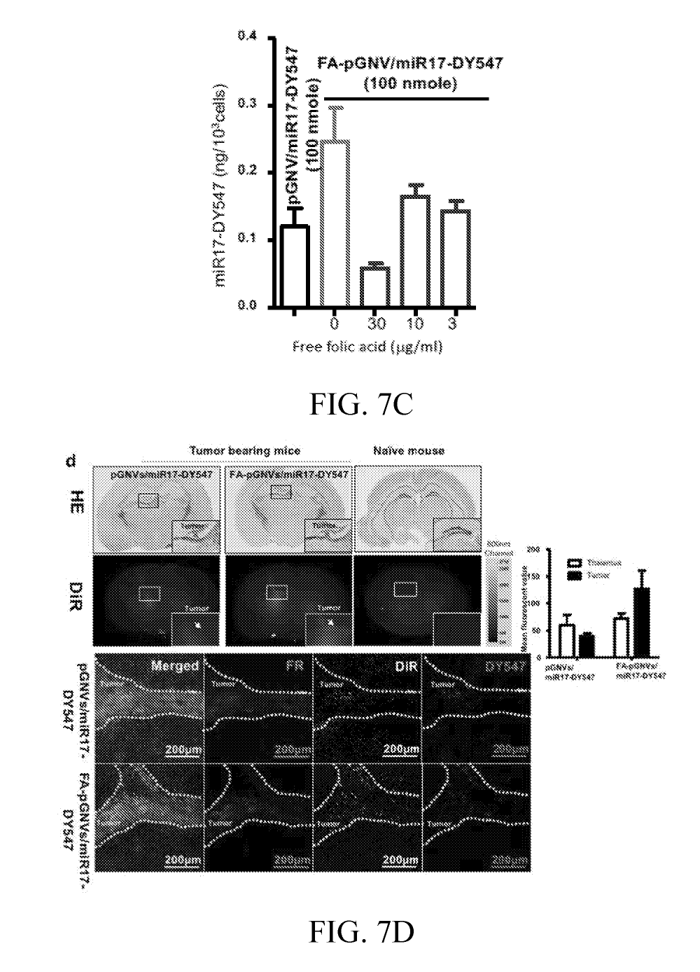

To evaluate the potential use of FA-pGNVs as a targeting vector to deliver therapeutic agents to brain tumor, the efficient uptake of FA-pGNVs by GL-26 brain tumor cells was first evaluated in in vitro cell culture. GL-26-luc cells were co-cultured with FA-pGNVs or pGNVs carrying Dylight547 fluorescent dye labeled RNA. The presence of FA-pGNV/RNA and pGNV/RNA in GL-26-luc cells was examined using confocal microscopy (FIG. 7A, top panel) and determined by quantitative analysis of the numbers of Dylight547 labeled RNA+cells. The results indicated that the majority of GL26 cells internalized the FA-pGNV/RNA. More than 80% of the GL-26 cells took up the FA-pGNV/RNA within 2 hours of co-culture in comparison with 20% of the GL-26 cells taking up pGNVs/RNA. The fact that FA coated GNVs have better transfection efficiency was also demonstrated in GL-26 cells transfected with Syto60 labeled RNA/PEI complexed with GNVs (FA-GNV/RNA-syto60) (FIG. 7A, bottom panel). The amount of RNAs accumulating in the cells continuously increased and reached a plateau at 6 h after transfection (FIG. 7B). Pre-mixing FA-pGNV/RNA with the free form of folic acid led to a reduction of RNAs accumulating in the GL-26 cells in a folic acid dose dependent manner (FIG. 7C). This suggests that the enhanced internalization of FA-pGNV/RNA is FA receptor mediated.

We next sought to determine whether FA-pGNV/RNA has an enhanced efficiency in targeting brain tumor cells in a mouse model. Biodistribution of DiR-labeled FA-pGNV/RNA was evaluated in mice using the Odyssey imaging system. For these studies, groups of mice bearing intracerebral tumors (FIG. 7D, top panel, and bottom panel, the first column from the right) were intranasally administrated DIR dye labeled FA-pGNV/miR17-DY547 or pGNV/miR17-DY547. The amount of DIR+FA-pGNV/miR17-DY547 or pGNV/miR17-DY547 present after administration was quantitatively analyzed. Imaging data showed a statistically significant increase in brain tumor (FIG. 7D, middle panel) associated photons in FA-pGNV/miR17-DY547-treated mice when compared to pGNV/miR17-DY547. This result is further supported by increased fluorescent DY547 labeled RNA signals detected in the brain tumor (FIG. 7D, bottom panel, second columns from right) and co-localized with GL-26 cells that have high density of folate receptors expressed (FIG. 7D, bottom panel, first column from left).

### Example 4

### Intranasal targeted delivery of miR17 encapsulated in FA-pGNVs inhibits GL26 tumor growth

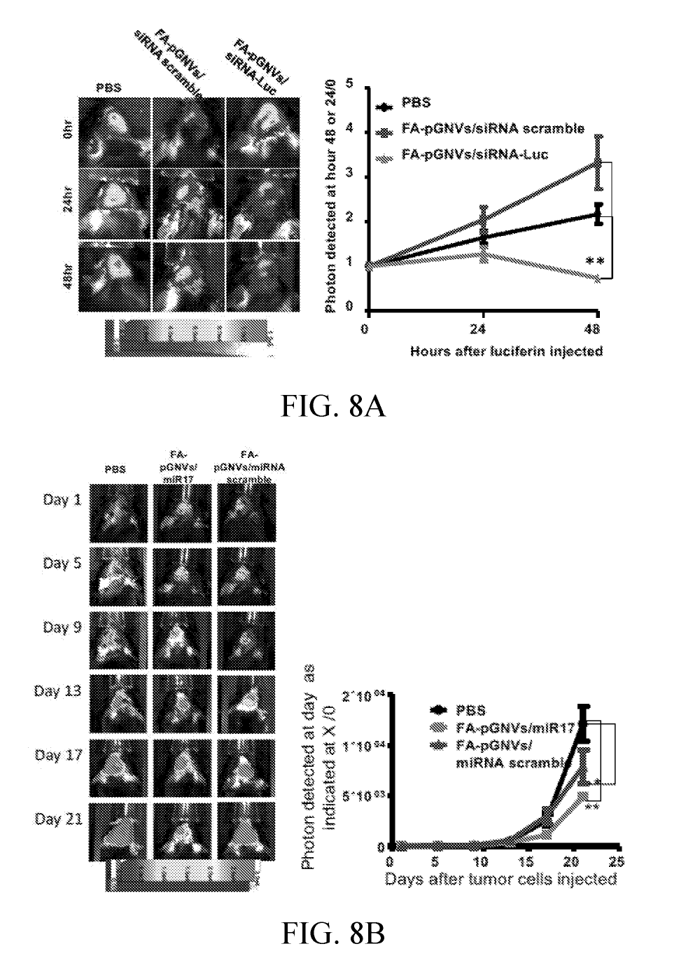

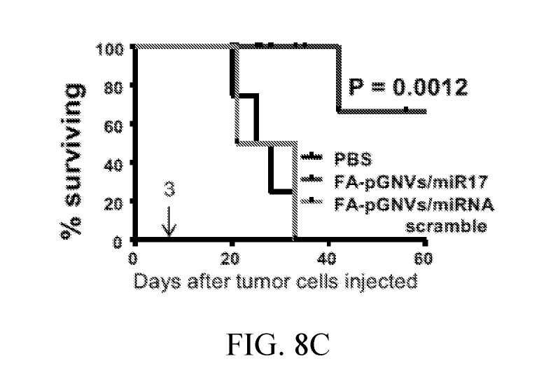

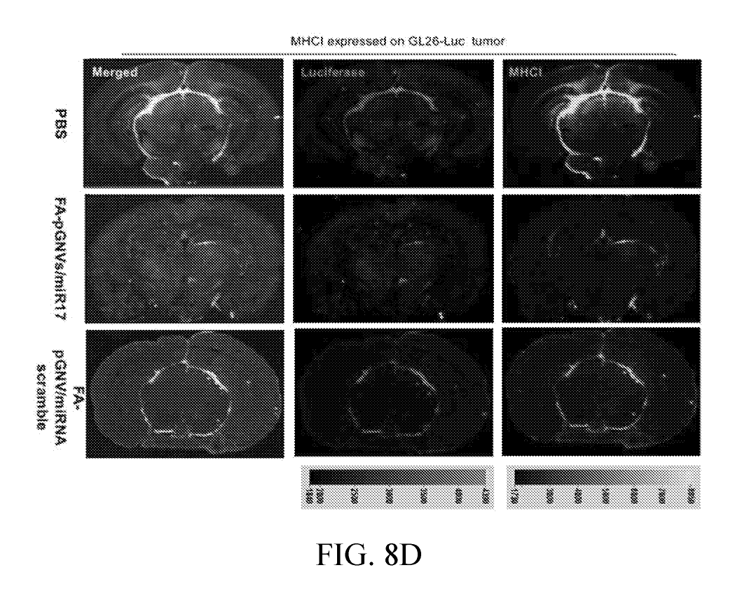

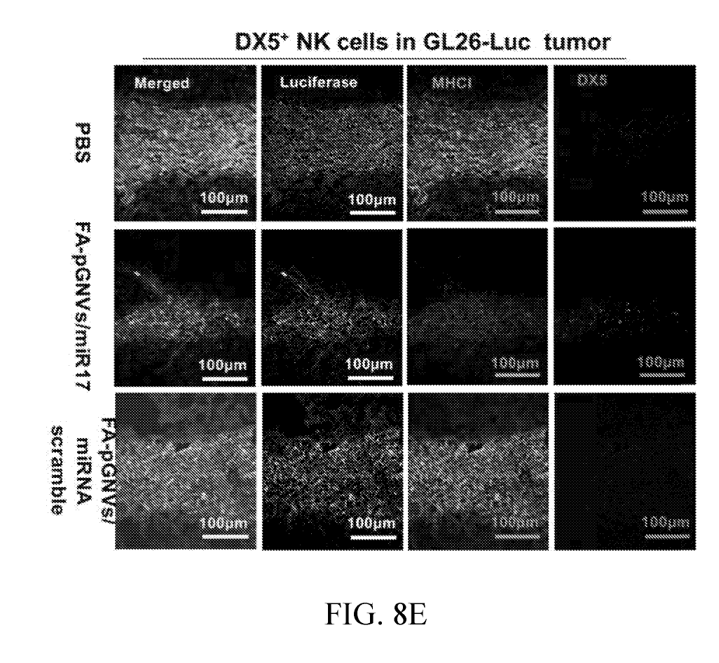

Finally, we determined whether miR17 carried by FA-pGNVs has a therapeutic effect in a mouse brain tumor model. We begin with testing whether RNA carried by the FA-pGNVs still has biological activity. To address this issue, we used a well-characterized siRNA that is directed against a luciferase reporter gene stably expressed in GL26-Luc cells. Luciferase siRNA or siRNA scramble (5 μg) carried by the FA-pGNVs was intranasally administrated to 15-day GL-26-luc tumor bearing mice. Imaging data showed a statistically significant decrease in brain-associated photons in FA-pGNVs/siRNA-Luc treated mice when compared to FA-pGNV/siRNA scramble treated mice (FIG. 8A) at 48 h.

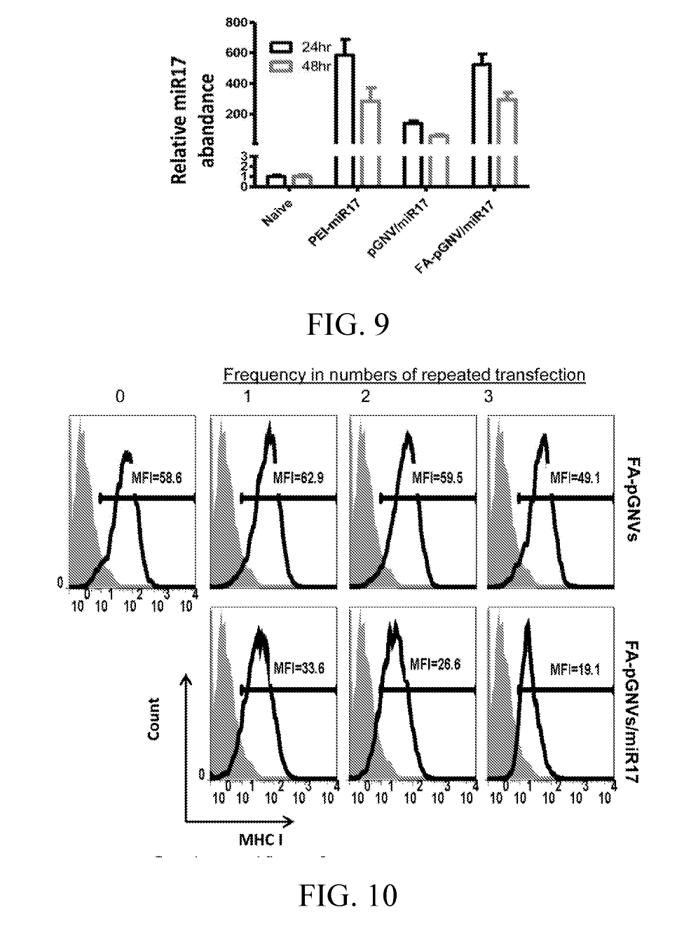

Our published data suggest that one of the miR17 targeted genes is MHC-1. miR17-mediated downregulation of MHC1 expressed on the tumor cells led to activation of NK cells and inhibited tumor growth. Therefore, we tested whether miR17 carried by FA-pGNVs can be delivered to GL-26 brain tumor and achieve a therapeutic effect. qPCR analysis indicated that FA-pGNVs are more efficient delivering miR17 to GL26 cells than pGNVs and the FA-pGNVs are stable 48 h after transfection (FIG. 9). FACS analysis further indicated that miR17 inhibits MHCI expression on GL-26 cells (FIG. 10). Next, we conducted in vivo delivery experiments of miR17 with FA-pGNVs targeting of mouse GL-26 brain tumor to determine the therapeutic effect of miR17. We treated groups of mice bearing intracerebral tumors with FA-pGNV/miR17, FA-pGNV/miRNA scramble, or PBS as a control. Mice were treated every three days for 21 days beginning on day 5 after the tumor cells were implanted. The amount of miR17 administered was based on the lack of any evidence of toxicity or behavioral abnormalities in the mice. Twenty-one days after tumor cells were injected imaging data showed a statistically significant decrease in brain-associated photons in FA-pGNV/miR17 treated mice when compared to controls (FIG. 8B). Survival times of PBS control and FA-pGNV/scramble miRNA animals ranged from 20 to 33 days. In contrast, FA-pGNV/miR17 treatment significantly prolonged the survival of mice to an average of 47.5 days (P<0.0012) (FIG. 8C). Although none of the FA-pGNV/miR17-treated animals exhibited evidence of toxicity or behavioral abnormalities during the treatment period, most of the FA-pGNV/miR17 treated surviving mice (8/12) were not tumor free on day 70; the on which all of mice were killed for evaluation of brain tumors by HE staining. To further investigate if the reduction of tumor cells in the brain is associated with induction of NK cells in the FA-pGNVs/miR17 targeted tumor, the numbers of luciferase expressed GL-26 tumor cells (FIG. 8D) and of NK cells in the GL-26 tumor (FIG. 8E) were determined. The results suggest that FA-pGNV/miR17 treatment led to increased numbers of DX5+NK cells in the GL-26 tumor (FIG. 8E). The induction of DX5+NK cells was also correlated with a decrease in the expression of MHCI+luciferase+ GL-26 tumor cells (FIG. 8E). Collectively, these data support the idea that FA-pGNV/miR17 is selectively taken up by GL-26 cells and subsequently inhibits the expression of1\41-1CI expressed on the GL-26 tumor cells, which triggers activation of NK cells to kill tumor cells.

Discussion of Examples 1-4

The lack of access to the brain is a major obstacle for central nervous system drug development. For example, a large number of drugs with therapeutic potential for treatment of brain related diseases are never pursued due to their inability to be delivered across the BBB in therapeutic concentrations. Although intranasal delivery provides a practical, noninvasive method for delivering therapeutic agents to the brain, the quantities of drug administered nasally that have been shown to be transported directly from nose-to-brain are very low. Although our results suggest that intranasal delivery of an anti-inflammatory agent such as curcumin, and the anti-Stat3 agent, JSI-124, provides a promising noninvasive approach for the treatment of brain inflammatory related diseases such as malignant gliomas, biosafety considerations and large scale production of mammalian cell-derived exosomes has been challenging. To meet this challenge, we recently developed fruit-based nanovectors made of lipids extracted from edible plant exosomes. Exosome-like nanoparticles from grapefruit naturally encapsulate small RNAs, and proteins. We have shown that grapefruit derived nanovectors (GNVs) are highly efficient for delivering a variety of therapeutic agents including drugs, DNA expression vectors, siRNA and antibody in mouse model studies without inducing toxicity.

Using GNVs for intranasal delivery of therapeutic agents has not been addressed. In these Examples, a GNV-based nanovector hyrided with polyethylenimine (PEI) (pGNV) was developed for effective intranasal delivery of miRNA to brain. The reason for using PEI as an enhancer for delivering nucleic acid is that PEI has a higher efficiency in carrying RNA and DNA. However, cationic polyplexes formed by PEI and nucleic acids are toxic and is due to the positive charge on the surface of the particles necessary for the binding of oligonucleotides. Positively charged PEI polyplexes are required for high efficient transfection; in the absence of the free net positive charge PEI polyplexes intracellular elimination of nucleic acids is faster. The toxicity of the PEI is reduced by making hybrid the PEI polyplexes with GNVs. Enhanced targeting was further achieved by coating pGNVs with the tumor targeting moiety, folic acid. This allowed for active targeting of cancer cells to potentiate the transfection efficiencies of brain cancer cells in vitro and in vivo. This study therefore provides an effective approach to overcome the efficiency-toxicity challenges faced with nonviral vectors. Additionally, this study provides insights into the design strategy of effective and safe vectors for cancer gene therapy.

More specifically, in the Examples above, the capability of a grapefruit-derived nano vector (GNVs) to carry miR17 for therapeutic treatment of mouse brain tumor is demonstrated. It is also shown that GNVs coated with folic acid (FA-GNVs) are enhanced for targeting the GNVs to a folate receptor positive GL26 brain tumor. Additionally, FA-GNVs coated polyethylenimine (FA-pGNVs) not only enhance the capacity to carry RNA, but the toxicity of the polyethylenimine is eliminated by the GNVs. Intranasal administration of miR17 carried by FA-pGNVs led to rapid delivery of miR17 to the brain that was selectively taken up by GL-26 tumor cells. Mice treated intranasally with FA-pGNV/miR17 had delayed brain tumor growth. These results demonstrate that this strategy may provide a noninvasive therapeutic approach for treating brain related disease through intranasal delivery.

Although the efficacy of using mammalian cell-derived exosomes as a delivery vehicle for intranasal delivery of therapeutic agents has been demonstrated in mouse models, biosafety considerations and large scale production of mammalian cell-derived exosomes has presented obstacles to their clinical use. The present study examined a novel approach for GNV-mediated intranasal delivery of RNA in general and therapeutic miR17 specifically to the brain tumor cells. Our results clearly indicate that RNA, including miR17, is effectively delivered to the brain by pGNVs without observable side effects. Furthermore, our study advances an approach for targeted delivery of therapeutic miR17.

In these studies we used folate acid coated pGNVs (FA-pGNVs) as proof of concept, to demonstrate enhanced targeting to GL-26 glioma tumor cells which express increased amounts of the folate receptor; which promoted much more substantial therapeutic benefits without inducing adverse side-effects. Like other liposomes, the folate ligand could be incorporated into the liposomal bilayer during pGNV preparation by mixing a lipophilic folate ligand with other GNV lipid components. The lipophilic anchor for the folate ligand can be either GNV phospholipid or cholersterol. The FA-pGNVs also avoids several of the problems such as the lack of tissue targeting specificity, toxicity and difficulty in scalability and production, the need for life-long monitoring for potential tumorigenesis and other adverse clinical outcomes that have arisen with conventional therapy vectors including PEI and DOTAP. Because FA-pGNVs do not cause these concerns they have great potential as targeted delivery vehicles, in particular, because production of GNVs is easily scaled up and the GNVs can be coated with a variety of targeting moieties. Since chemically synthesized nanovectors are known to induce toxicity, which is a major obstacle for clinical use, the approach combining PEI and GNVs as we demonstrated in this study could apply to nanotechnology in general to overcome the potential toxicity for clinical application.

Our data presented in this study show that miR17-mediated induction of NK cells through down-regulation of MHCI expressed on the GL-26 tumor cells is one of the mechanisms underlying the therapeutic effects; other mechanisms cannot be excluded for contributing to the anti-tumor growth as miR17 is a pleiotropic miRNA like other miRNAs that can target multiple pathways. From a therapeutic standpoint, an appealing property of miRNAs as therapeutic agents is their capacity to target multiple genes, making them extremely efficient in regulating distinct biological processes in the context of a network. Genes involving such a network are dysregulated during the development of cancer. Therefore, developing therapeutic strategies to restore homeostasis by delivery of miRNA would be more efficient than targeting individual genes or proteins. In addition, GL26 cells may be not the only cells targeted by FA-pGNVs. The biological effects of other cells, particularly FA positive infiltrating immune cells, including myeloid cells on the inhibition of brain tumor progression may also be involved and needs to be further studied.

For more efficient therapeutic outcomes, enhanced selectivity or targeting of nano-vector based delivery vehicles is required to ensure targeting of tumor cells and not healthy normal cells. The enhanced permeability and retention (EPR) effect in combination with modification of the vector by coating with a targeting moiety have been extensively studied for improving targeting efficiency. However, it is unlikely that 100% of the tumor cells can be targeted. In addition most of delivery vectors are made of foreign material which is immunogenic and cannot be given repeatedly. In contrast, non-immunogenic GNVs can be used to carry therapeutic agents including anti-tumor and/or to stimulation of immune response, simultaneously. This will lead to not only to a reduction in tumor size but also the possible elimination of residual tumor cells that can be chemo-resistant.

In this study, we found rapid movement of GNVs into the brain within 1.5 hour of intranasal administration. This finding is consistent with the results generated from mammalian cell EL4-derived exosomes. Collectively, fast and selective homing to the brain of FA-GNVs warrants further exploration for their ability to carry of other types of biological cargo including drugs, therapeutic antibodies, and oncolytic viruses which selectively replicate in tumor cells.

Although our findings demonstrate the potential for using GNVs as a novel, noninvasive delivery vehicle to target therapeutic agents to the brain, more fundamental studies are required, such as the mechanism underlying the GNV mediated high intranasal transporting efficiency versus poor transporting efficiency of DOTAP. Additional research is also necessary to study the mechanism of GNVs trans-location from the nasal cavity to the brain and identify the route by which GNVs travel to the olfactory bulb and ultimately throughout the nervous system. In addition, we noticed that DIR labelled pGNVs do not signal intensity equal to Syto60 labelled pGNVs (FIG. 3D). This could be due to having multiple copies of Syto60 labeled RNA that are complexed with one copy of GNV. Therefore, the signal generated from Syto60 labeled RNA is the more intense signal. It is also possible that Syto60 less effected (more stable) during trafficking from the nose to the brain than DiR dye which would explain the higher signal intensity.

Statistical analysis. Survival data were analyzed by log rank test. Student's t-test was used for comparison of two samples with unequal variances. One-way ANOVA with Holm's post hoc test was used for comparing means of three or more variables.

Materials and Methods for Examples 5-9.

FISH (fluorescence in situ hybridization). To visualize biotin conjugated miR-18a in the liver, tissue sections were deparaffinized and rehydrated. After permeabilization by adding 1% triton X-100, tissue sections were incubated in PBS containing 5 mg/ml of lysozyme at 37° C. for 20 min. Following a pre-incubation at 46° C. for 1 h in hybridization buffer (900 mM NaCl; 20 mM Tris-HCl, pH8.0; 1 mM EDTA, pH8.0), tissues were hybridized with 0.1 μM of Alexa Fluor® fluorescent conjugated streptavidin at 46° C. overnight. After dehydrating the tissue sections in a graded ethanol series, i.e., 70%, 80%, 95%, 100% ethanol, nuclear chromatin was stained with 4′, 6-diamidino-2-phenylindole (DAPI) and the tissues were analyzed using confocal laser scanning microscopy.

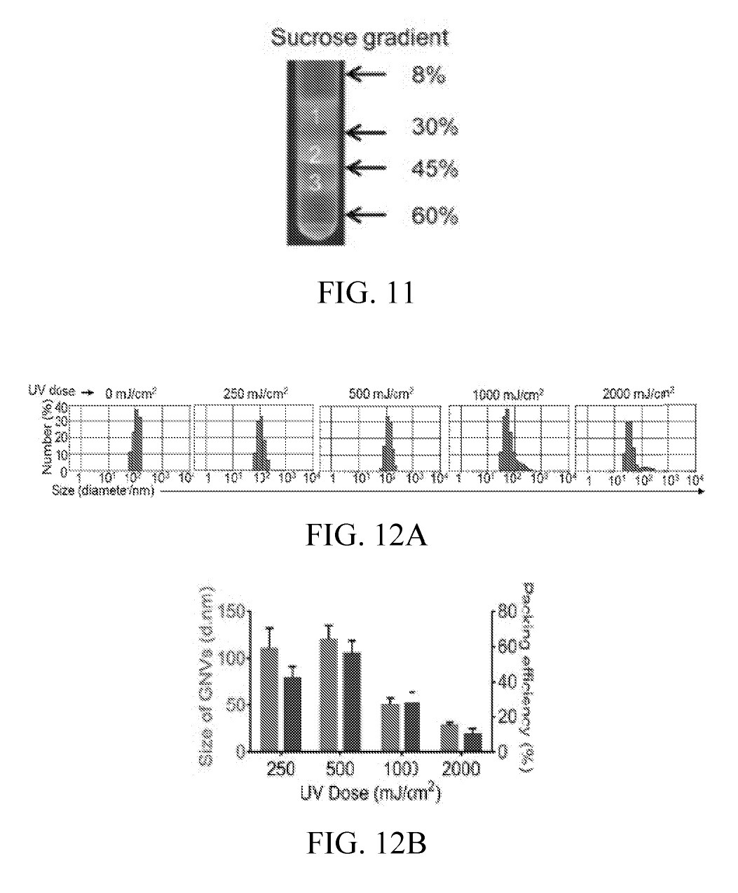

Preparation and characterization of optimized GNVs (OGNVs). Grapefruit derived lipids were prepared, as previously described. In brief, the sucrose gradient purified grapefruit nanoparticles were harvested from the 30%/45% interface (FIG. 11). The lipids were extracted with chloroform and dried under vacuum. The concentration of lipids was measured using the phosphate assay as described. To generate OGNVs, 200 nmol of lipid was suspended in 200-400 μl of 155 mM NaCl with 10 μg of RNA. After UV irradiation at 500 mJ/cm2 in a Spectrolinker (Spectronic Corp.) and bath sonication (FS60 bath sonicator, Fisher Scientific) for 30 min, the pelleted particles were collected by centrifugation at 100,000g for 1 h at 4 ° C. The size and zeta potential distribution of the particles was analyzed using a Zetasizer Nano ZS (Malvern Instrument, UK).

Labeling RNA in OGNV with Exo-GLOW. RNA in OGNVs was labeled with Exo-GLOW™ Exosome Labeling Kits (Cat # EXOR100A-1, System Biosciences) in accordance with the manufacturer's instructions. 10 μl of resuspended OGNVs with encapsulated RNA was diluted into 500 μl of PBS with 50 μl of 10× Exo-Red and incubated at 37° C. for 10 min. To stop the labeling reaction, 100 μl of the ExoQuick-TC reagent was used and the reaction was placed on ice for 30 min. After washing by centrifugation at 13,000 rpm for 3 min, OGNVs were resuspended and were assessed for fluorescence intensity with an excitation maximum at 460 nm and emission maximum shift to 650 nm. Details of other methods used in this study are described in the supplemental experimental procedures.

Mouse Model study. 8- to 12- week-old female BALB/C mice, Interferon gamma (IFNγ) knockout mice and severe combined immunodeficiency (SCID) mice were purchased from The Jackson Laboratory (Bar Harbor, Me.) and housed under specific pathogen-free conditions. Animal care was performed following the Institute for Laboratory Animal Research (ILAR) guidelines and all animal experiments were done in accordance with protocols approved by the University of Louisville Institutional Animal Care and Use Committee (Louisville, Ky.). The mice were acclimated for at least 1 week before any experiments were conducted.

Animal model of colon cancer with liver metastasis. Mice were anaesthetized with a mixture of ketamine and xylazine and 1×10⁶ CT26 colon cancer cells were administered via intra-splenic injection as previously described(1). At day 3 after intra-splenic injection, 200 nM OGNVs packing 2 nM of miR-18 was administrated to mice by tail veil injection, three times per week for 2 weeks. On day 14 mice were sacrificed and various organs were removed for examinations.

Liver macrophage depletion. Mice were injected with approximately 110 mg/kg of clodronate liposomes (FormuMax Scientific Inc.) i.p. or an equal volume of PBS liposomes. The injection was repeated three days later and experiments were performed 4 days after the first injection.

Antibodies and reagents. The following monoclonal antibodies (eBioscience) were used for flow cytometry: F4/80 (17-4801-82), anti-CD3 (46-0032-82), anti-Dx5 (17-5971-82), anti-IL-12 (12-7123-82), anti-CD80 (12-0801-82), anti-CD86 (11-0862-85), anti-IFNγ (11-7311-82). The following monoclonal antibodies purchased from Biolegend were used for flow cytometry: anti-CD3 (100206), anti-Dx5 (103503), anti- anti-MHCII (107624), anti-IL-12 (505205), anti-CD80 (122007), and anti-CD86 (105027).

Cell culture. The BALB/c syngeneic CT26, undifferentiated colon cancer cell line, and RAW264.7, murine macrophage cell line (American Type Culture Collection, Rockville, Md.) were grown at 37° C. in 5% CO2 in Dulbecco's Modified Eagle's medium (DMEM) medium and RPMI 1640 medium (Gibco), respectively, supplemented with 10% heat-inactivated fetal bovine serum (FBS), 100 U/mL penicillin, and 100 μg/mL streptomycin.

Flow cytometry. Liver and spleen from mice were thoroughly dissected and gently pressed through nylon cell strainers (70 μm in diameter, Fisher Scientific) to obtain single-cell suspensions in RPMI-1640 containing 5% FBS. Hepatocytes were removed from liver-cell suspensions by colloidal silica particles (Percoll) gradient centrifugation in phosphate-buffered saline. Erythrocytes in liver and spleen-cell suspensions were then removed using Ammonium-Chloride-Potassium (ACK) lysing buffer (0.15 M NH₄Cl, 10 mM KHCO₃, 0.1 mM EDTA). Washed cells were stained for 40 min at 4° C. with the appropriate fluorochrome-conjugated antibodies in PBS with 2% FBS. To detect intracellular antigens, washed cells were incubated in diluted Fixation/Permeabilization solution (eBioscience Cat# 005123) at 4° C. for 30 min. Characterization and phenotyping of the various lymphocytes subsets from liver or spleen were performed by flow cytometry. Data were acquired on BD FACS Canto (BD Biosciences, San Jose, Calif.) and were analyzed using FlowJo software (Tree Star Inc., Ashland, Oreg.).

Intracellular cytokine production. Lymphocyte preparations were stimulated for 6 h with PMA (phorbol 12-myristate 13-acetate; 1 ng/ml; Invitrogen) and ionomycin (1 μM; Invitrogen), LPS (10 μg/ml), or GalGer (10 ng/ml) in the presence of brefeldin A (5 μg/ml ; Invitrogen). Cells were then stained for markers of NKT cells, NK cells, and T cells with anti-CD3 and anti-Dx5. The cells were fixed and permeabilized with fixation and permeabilization buffers (BD Biosciences) and intracellular IL-12, IFN-γ and TGFβ were stained and FACS-analyzed.

Site-directed mutagenesis within the IRF2 promoter. We utilized two algorithms that predict the mRNA targets of miRNAs, TargetScan (http://www.targetscan.org) and microRNA (http://www.microRNA.org), and Pictar (http://pictar.mdc-berlin.de/. IRF2 was selected by both online tools with strong conserved 3′ untranslated region (3′ UTR) sites. To determine the ability of miR-18a to target the 3′UTR-IRF2 activity, a luciferase reporter containing 1,234 bp of the IRF2 3′UTR in the pEZX-MT01 vector was purchased from GeneCopoeia (Cat# MmiT027452-MT01, Rockville, Md.). The mutant of IRF2 3′UTR was generated with the oligonucleotide primer IRF2-Mut, which was designed to specifically disrupt putative IRF2 at its 3′ UTR site. Q5® Site-Directed Mutagenesis Kit (New England Biolabs, MA, USA) was used in conjunction with specific primers (Table 1) to introduce IRF2 3′ UTR mutations in the pEZX-MT01 construct according to the manufacturer's instructions. After mutant strand synthesis and ligation, resultant plasmids were introduced into E. coli and transformants were selected using kanamycin resistance. Further DNA sequence of mutant was confirmed by DNA sequencing.

TABLE 1
Primer sequences used for quantitative Real-Time PCR (qPCR) of mRNA.

| | Primers | Forward (5′-3′) | Reverse (5′-3′) |
|---|---|---|---|
| qPCR | mm-TGFβ | (SEQ ID NO: 1) | (SEQ ID NO: 2) |
| | | CAGGCGTCAGCGTATTCC | CCTTCCCTACCCGTCCAA |
| | mm-INFγ | (SEQ ID NO: 3) | (SEQ ID NO: 4) |
| | | ACTGGCAAAAGGATGGTGAC | TGAGCTCATTGAATGCTTGG |
| | mm-MHCII | (SEQ ID NO: 5) | (SEQ ID NO: 6) |
| | | GTCTCAGACTGTAAGACCTGAATG | GCTGAGGTGGTGGATACAATAG |
| | mm-IL-12 | (SEQ ID NO:?+0) | (SEQ ID NO: 8) |
| | | CAATCACGCTACCTCCTCTTT | ACCATGTCATCTGTGGTCTTC |
| | mm-SMAD2 | (SEQ ID NO: 9) | (SEQ ID NO: 10) |
| | | TCACAGACCCATCAAACTCG | ACTCAGCAAACACTTCCCC |
| | mm-ESR1 | (SEQ ID NO: 11) | (SEQ ID NO: 12) |
| | | AACCGCCCATGATCTATTCTG | AGATTCAAGTCCCCAAAGCC |
| | mm-ESR2 | (SEQ ID NO: 13) | (SEQ ID NO: 14) |
| | | ACGAAGTAGGAATGGTCAAGTG | GGTTCTCTTGGCTTTGTTCAG |
| | mm-IRF1 | (SEQ ID NO: 15) | (SEQ ID NO: 16) |
| | | GAAGGGAAGATAGCCGAAGAC | TCTGGTTCCTCTTTGCAGC |
| | mm-IRF2 | (SEQ ID NO: 17) | (SEQ ID NO: 18) |
| | | GATTTCTCCTGTGTCTTCCTACG | TTCCGTGTCCCCATGTTG |
| | mm-LEF | (SEQ ID NO: 19) | (SEQ ID NO: 20) |
| | | AGAACACCCTGATGAAGGAAAG | GTACGGGTCGCTGTTCATATT |
| | mm-TCF | (SEQ ID NO: 21) | (SEQ ID NO: 22) |
| | | GGTTCACCCACCCATCCT | TTGCGGGCCAGTTCATAG |
| | mm-AXIN2 | (SEQ ID NO: 23) | (SEQ ID NO: 24) |
| | | AGCCTAAAGGTCTTATGTGG | ATGGAATCGTCGGTCAGT |
| | mm-Wnt7a | (SEQ ID NO: 25) | (SEQ ID NO: 26) |
| | | GGACGAGTGTCAGTTTCAGT | CACAGTCGCTCAGGTTGC |
| Mutant | Mutantgenesis | (SEQ ID NO: 27) | (SEQ ID NO: 28) |
| | | AATGTCGCGGGCGGAGGCTGACCCG | AAGTGCTTCAAGATCCGGGTCA |
| | | GATCTTGAAGCACTT | GCCTCCGCCCGCGACATT |
| | Sequencing of | (SEQ ID NO: 29) | (SEQ ID NO: 30) |
| | mutant | CCTCAAGTTCAAGGACCAACA | GCTGTGAAGGAGAGCAAGATTA |

Transient transfection and luciferase reporter assay. Murine macrophage RAW264.7 cells were plated in 24-well plates at a density of 3.0×10⁴ cells/well in antibiotic free RPMI-1640 medium supplemented with 10% FBS. 100 ng of pEZX-MT01 or mutant luciferase reporter were transfected using FuGENE HD Transfection Reagent (Roche Applied Science, Indianapolis, Ind.) with 10 pmol of mimic mmu-miR-18a and Opti-MEM® Reduced Serum Medium (Invitrogen, Carlsbad, Calif.). For all reporter assays, the cells were harvested 48 h post-transfection using Promega's Passive Lysis buffer. The activities of luciferase in cell lysates were determined using the Dual-Luciferase Reporter Assay System (Promega). Relative expression (fold change) was determined by dividing the averaged normalized values from mock transfection. Values were averaged as indicated in the Figure legends.

Labeling OGNT7s with PKH67. OGNVs were labeled with PKH67 Fluorescent Cell Linker Kits (Sigma) in accordance with the manufacturer's instructions. OGNVs were suspended in 250 μl of Diluent C with 1 μl of PKH67 and mixed with 250 μl of dye solution for subsequent incubated with an equal volume of 1% BSA for 1 min at 22° C. After centrifugation for 5 minutes at 13,000 rpm, 20 μl of resuspended labeled OGNVs were loaded on a slide for assessment of viability using confocal microscopy (Nikon).

Quantitative Real-Time PCR (qPCR) analysis of miRNA and mRNA expression. Total RNA was isolated from lymphocyte cells with a miRNeasy mini kit (Qiagen) and reverse-transcribed using a miRNA reverse transcription kit (Qiagen). Mature miR-18a expression was quantified by quantitative real-time PCR (qPCR) using a miScript II RT kit (Qiagen) and miScript SYBR Green PCR Kit (Qiagen) with Qiagen predesigned primers. All kits were used according to the manufacturer's instructions. U6 transcript was used as an internal control to normalize RNA input. For analysis of IL-12, IFNγ, MHCII, TGFβ, IRFLIRF2, Smad2, ESR1, ESR2 mRNA expression, 1 μg of total RNA was reverse transcribed by SuperScript III reverse transcriptase (Invitrogen) and quantitation was performed using primers (Eurofin) with SsoAdvanced™ Universal SYBR Green Supermix (BioRad) and β-actin was used for normalization. The primer sequences are listed in Supplementary table 1. qPCR was run using BioRad CFX96 qPCR System with each reaction run in triplicate. Analysis and fold change were determined using the comparative threshold cycle (Ct) method. The change in miRNA or mRNA expression was calculated as fold-change.

Western blotting. Cells were treated as indicated in individual Figure legends and whole cell extracts (WCE) were prepared in modified RIPA buffer (Sigma) with addition of protease and phosphatase inhibitors (Roche). Western analysis was performed and quantitated as described(1). Proteins were separated by 10% SDS-PAGE and transferred to PVDF membranes (Bio-Rad Laboratories, Inc., Hercules, Calif.). Dual color precision protein MW markers (BioRad) were separated in parallel. Antibodies were purchased as follows: IRF2 (sc-498), α-tubulin (sc-8035), from Santa Cruz Biotechnology (Santa Cruz, Calif.) and IFNγ (ab9657, Abcam). The secondary antibodies conjugated to Fluors Alex-488 or Alex-594 were purchased from Invitrogen (Eugene, Oreg.). The bands were visualized on the Odyssey Imager (LiCor Inc, Lincoln, Nebr.).

Histological Analysis. Tissues were fixed with buffered 10% formalin solution (SF93-20; Fisher Scientific, Fair Lawn, N.J.) overnight at 4° C. Dehydration is achieved by immersion in a graded ethanol series, 70%, 80%, 95%, 100% ethanol for 40 min each. Tissues were embedded in paraffin and subsequently cut into ultra-thin slices (5 um) using a microtome. Tissue sections were stained with hematoxylin and eosin, and slides were scanned with an Aperio ScanScope. For frozen sections, tissues were fixed with periodate-lysine-paraformaldehyde (PLP) and dehydrated with 30% sucrose in PBS at 4° C., overnight. Tissue sections were stained with primary Ab in PBS/5% BSA (1:200) for 2 h and secondary Ab in PBS/5% BSA (1:800) for 30 min. 4′,6-Diamidino-. 2-phenylindole dihydrochloride (DAPI) was used for nuclear stain. Human colon cancer tissues slides, metastatic tissue and adjacent normal tissue were purchased from US Biomax Inc (Rockville, Md., Cat# C0702).

### Example 5

### Optimization of Efficiency of OGNVs for Encapsulating RNA

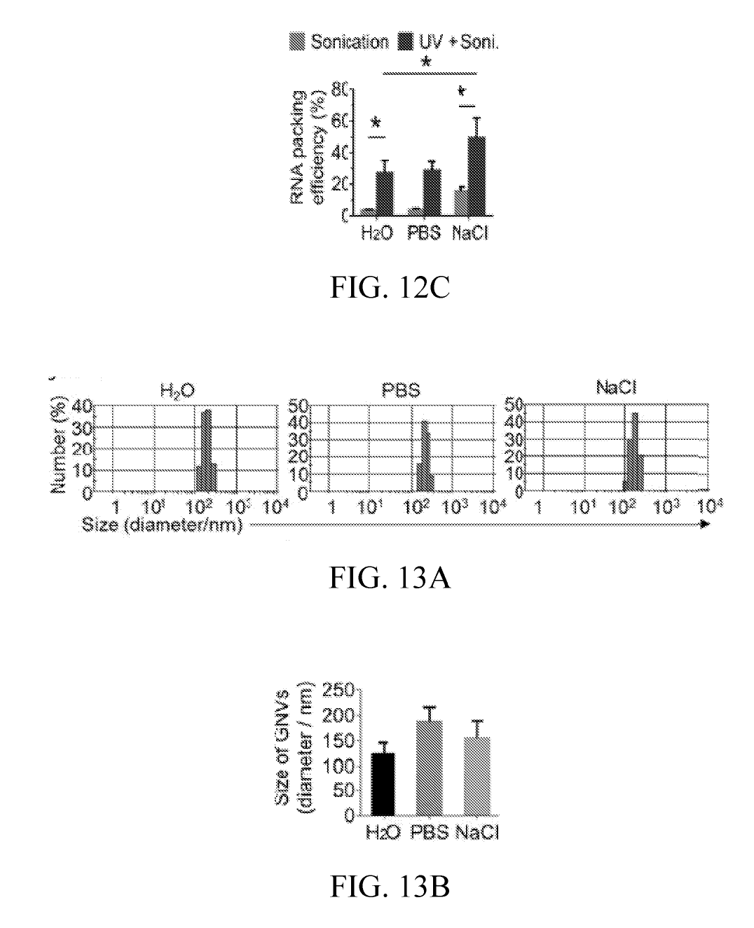

We first tested whether the efficiency of OGNVs for encapsulating RNA in general can be increased by Ultraviolet (UV) cross-linking lipids extracted from grapefruit nanoparticles with RNAs extracted from CT26 cells. Lipids extracted from sucrose gradient purified grapefruit nanoparticles (FIG. 11) and cellular RNA were mixed and exposed to different doses of UV light (254 nm) using a Spectrolinker. The results showed that lipids pre-exposed to UV radiation at 250 millijoules seconds per cm2 (mJ/cm2) and 500 mJ/cm2 reassembled into OGNVs with a diameter of 110.7±22.5 nm (means ±standard error of the mean (SEM)) and 120.6±15.7 nm, respectively (FIG. 12A). Both doses of UV radiation resulted in an increased efficiency of encapsulation for RNA from 5.5±2.2% to 28.2±4.8% and 30.6 ±4.5%, respectively (FIG. 12B). However, further increasing the dose of UV (1,000 mJ/cm2 and 2,000 mJ/cm2) resulted in decreasing the encapsulation efficiency of RNA.

Next, we tested whether neutralizing negative charges of the RNAs might further enhance the efficiency of encapsulation of RNA in OGNVs. OGNVs were assembled by sonication of grapefruit nanoparticle-derived lipids with RNA pre-dissolved in H2O, phosphate buffered saline (PBS, pH 7.4), and 155 mM sodium chloride (NaCl). Using 155 mM NaCl caused a 4.3-fold and 3.9-fold more efficient encapsulation of RNA than H2O and PBS, respectively (FIG. 12C). Furthermore, an additive effect was observed when NaCl was combined with UV radiation (FIG. 12C). The efficiency of encapsulation of RNA when placed in NaCl and exposed to UV radiation was increased markedly in comparison with H2O combined with UV exposure (49.6% vs 27.32%) or PBS combined with UV exposure (49.6% vs 28.62%). Collectively, the combination of UV radiation (500 mJ/cm2) and NaCl (155 mM) provides optimal conditions for enhancing RNA encapsulation efficiency in OGNVs. Henceforth we refer to the nanovectors made under these conditions as optimized-GNVs (OGNVs).

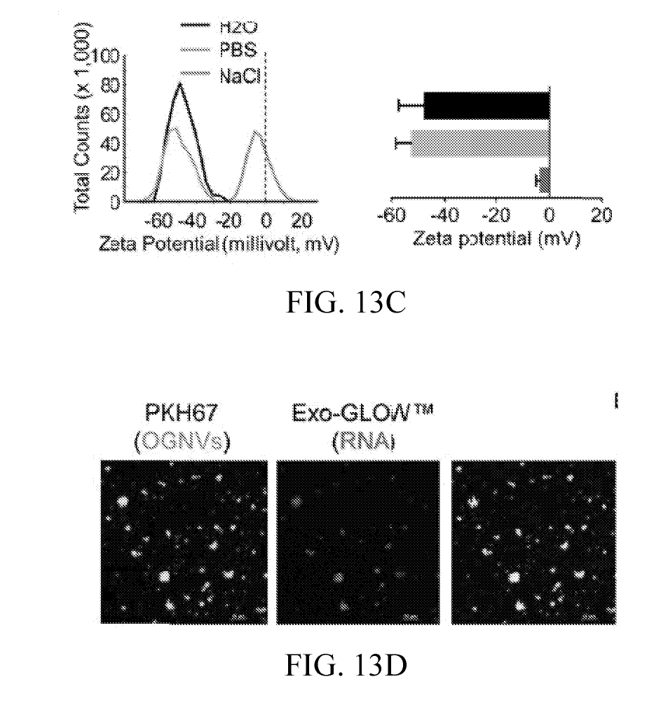

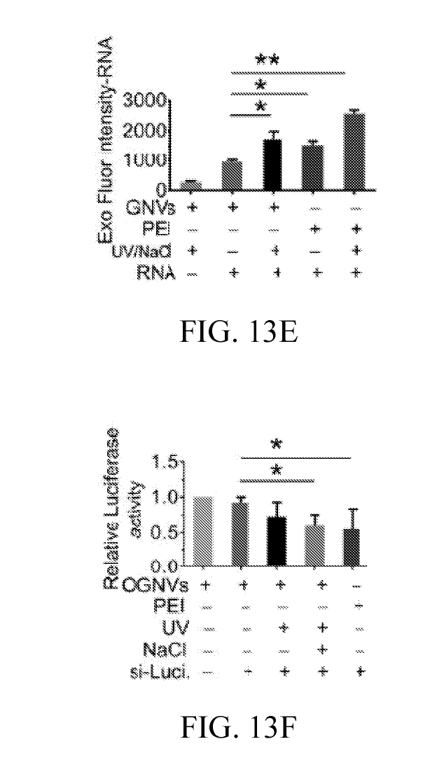

To determine whether UV radiation and NaCl have an effect on the functional characteristics of RNA encapsulated in OGNVs, we evaluated the size (FIG. 13A-B) and potential distribution (FIG. 13C) of OGNVs using a Zetasizer Nano ZS. With UV radiation, the average diameter of the OGNVs was 156±33 nm in NaC1, in comparison with 125±22 nm in H2O, and 188±28 nm in PBS. Zeta potential analysis revealed that OGNVs in H2O displayed a negative charge of −47.6±−9.61 mV. A NaCl concentration of 155 nM remarkably neutralized the charge of OGNVs to −3.4±1.7 mV (p<0.01), but PBS did not change the charge of OGNVs. Taken together, these data suggest that NaCl treatment of RNA not only increases encapsulation in OGNVs but alters the charge of OGNVs from strongly negative to weakly negative without dramatically affecting the size of the OGNVs.

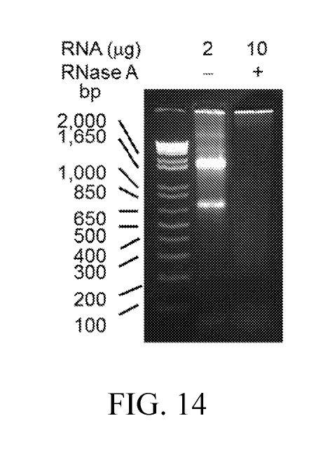

To further determine whether RNA has been encapsulated in the OGNVs or is located on the surface of OGNVs, OGNVs carrying Exo-GLOW (red) labeled RNA were digested with ribonucleases (RNase). Fluorescence analysis using confocal microscopy revealed RNA was still co-localized with OGNVs after RNase treatment (FIG. 13D-E). Furthermore, without detergent extraction, OGNV RNA was resistant to RNase digestion when OGNVs were kept at 4° C. for 7 days; whereas after extraction from OGNVs, the RNA without encapsulation in OGNVs was degraded by RNase (FIG. 14). Collectively, these results suggest that potentially therapeutic RNA can be encapsulated into OGNVs. Following this we determined whether UV treatment of OGNVs has an effect on the biological activity of encapsulated RNA. To address this concern, 20 μg of luciferase siRNA encapsulated in the OGNVs was transfected into U-87 MG-luc, a luciferase positive glioblastoma cell line which stably expresses the firefly luciferase gene. Assessment of luciferase activity with the Dual-Luciferase Reporter Assay System revealed that a similar activity of luciferase siRNA was demonstrated in the U-87 MG-luc cells transfected with OGNVs (40%) and polyethylenimine (PEI) (45%) (FIG. 13F), a commercial RNA delivery agent.

### Example 6

### miR-18a Encapsulated in OGNVs (OGNVs-miR18a) Induces M1 Kupffer Cells

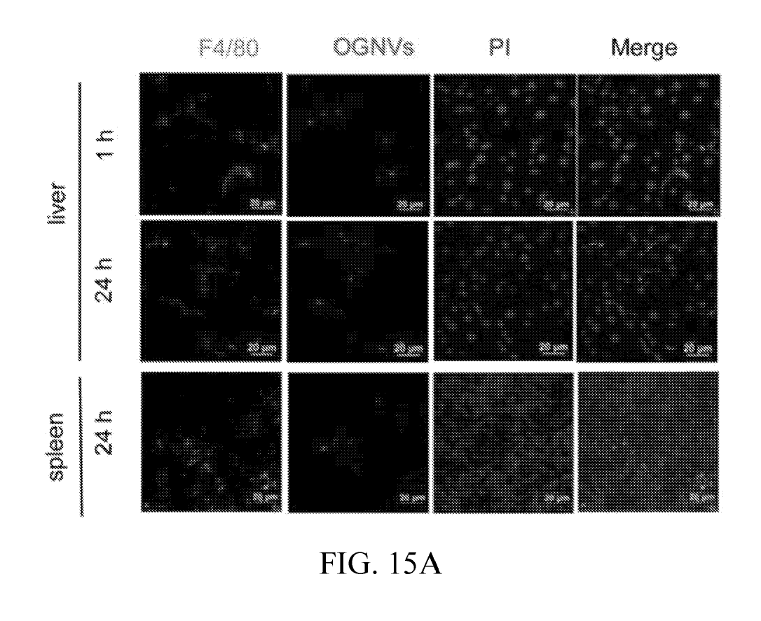

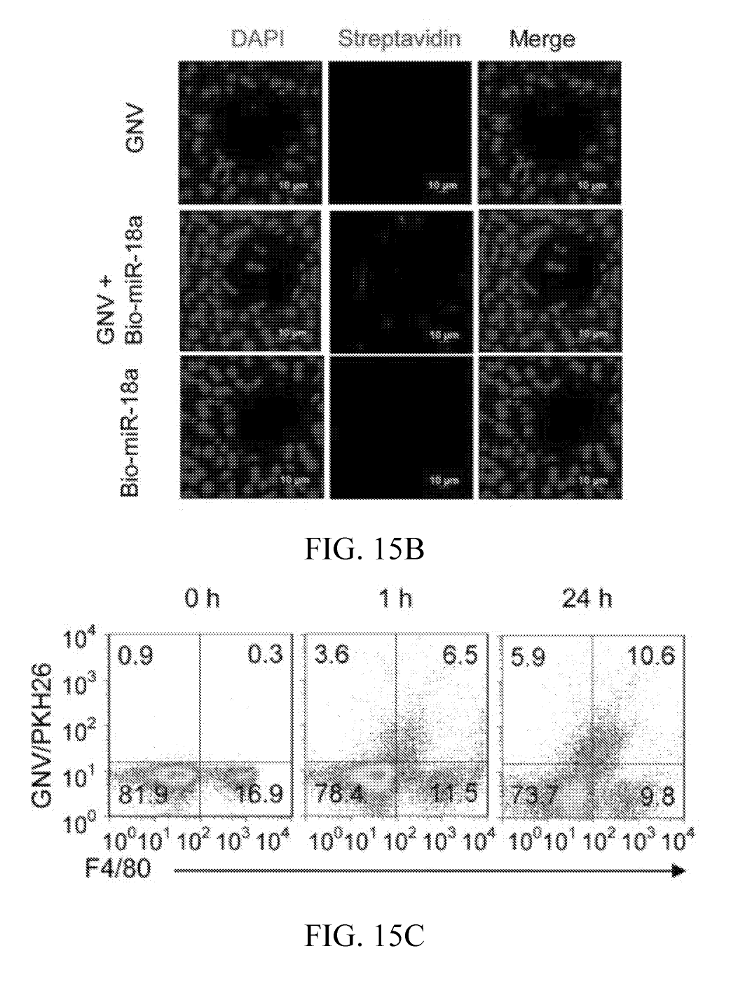

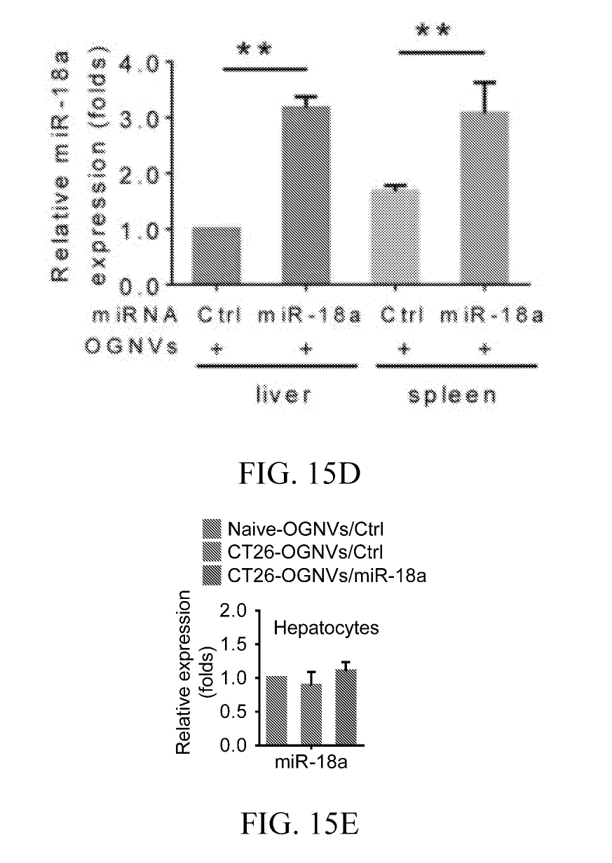

Liver KCs (FIGS. 15A-D) but not hepatocytes (FIG. 15E) take up OGNVs carrying miR18a after a tail vein injection. KCs represent 80-90% of all tissue macrophages in the entire body, play a major role in the capture and clearance of foreign material, are important antigen presenting cells (APCs), and express MHC I, MHC II and costimulatory molecules needed for activation of immune cells. Collectively, these features of liver KCs prompted us to test whether GNVs can be used as a vehicle for delivery of therapeutic agents for treatment of liver related disease through the mechanism of immunomodulation of Kupffer cells. Therefore, we set out to determine whether miR18a delivered by OGNVs has a biological effect on liver metastasis of colon cancer as an example.

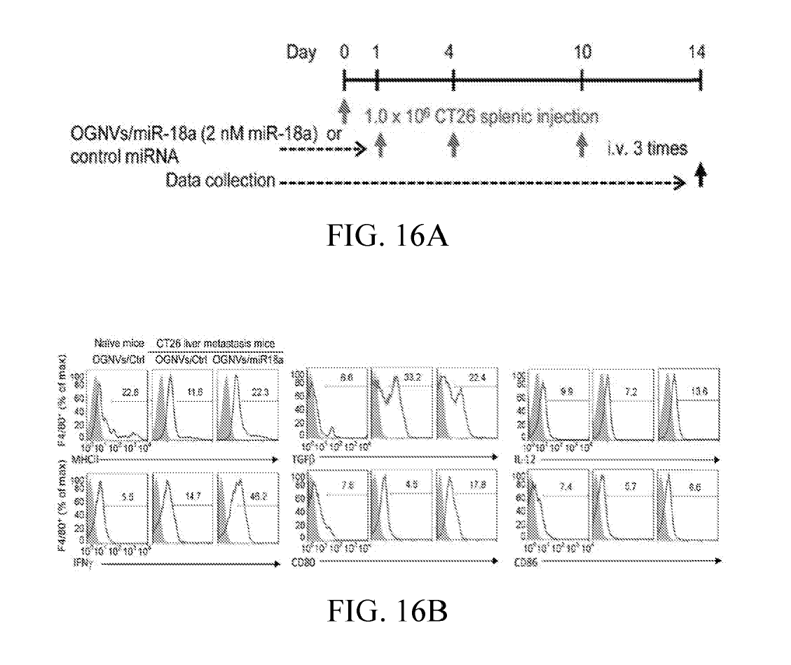

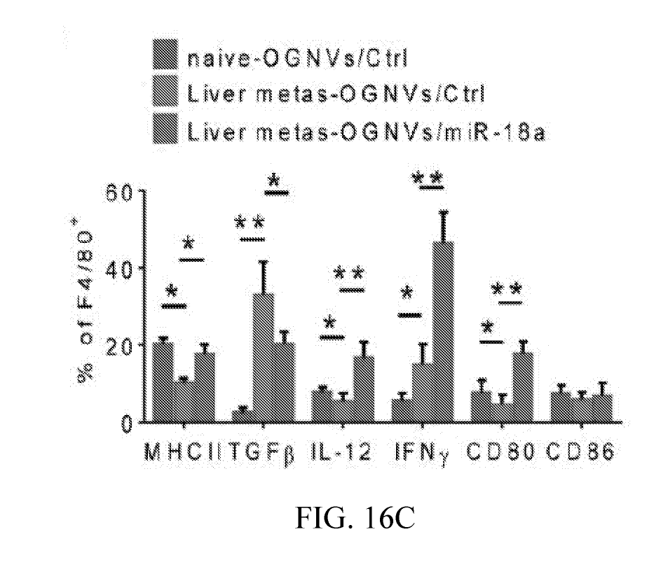

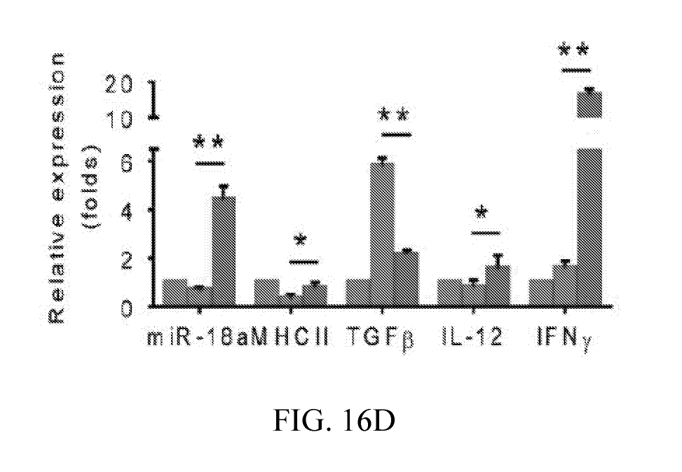

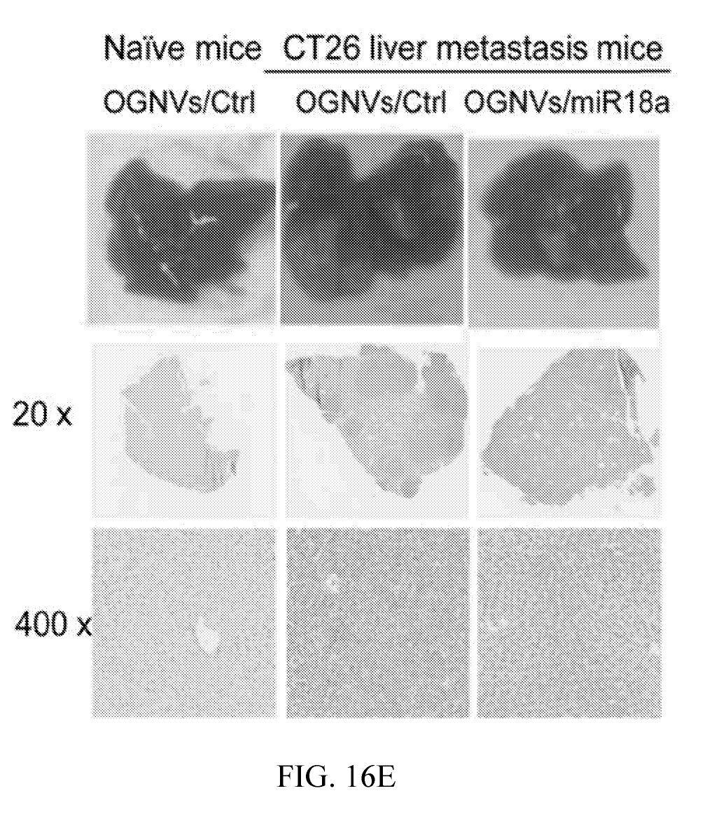

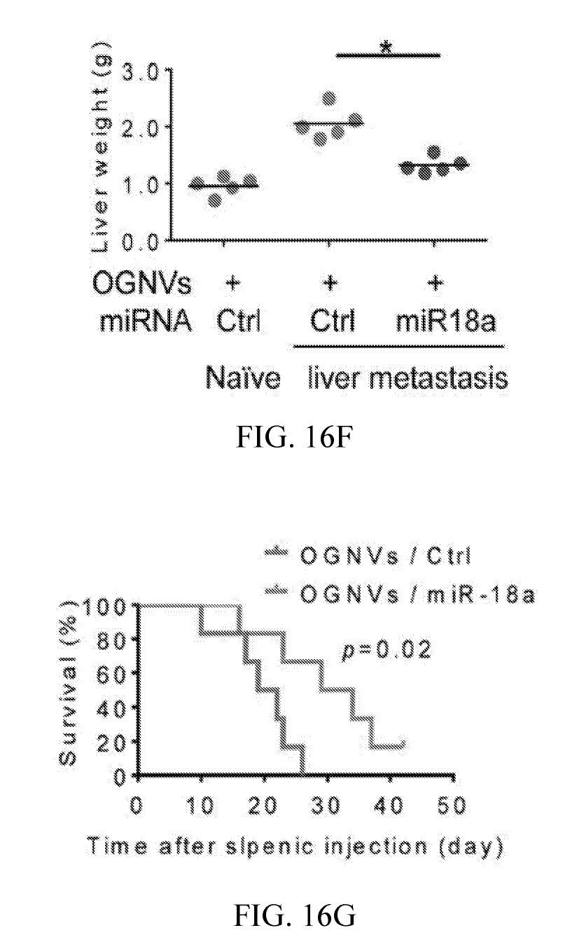

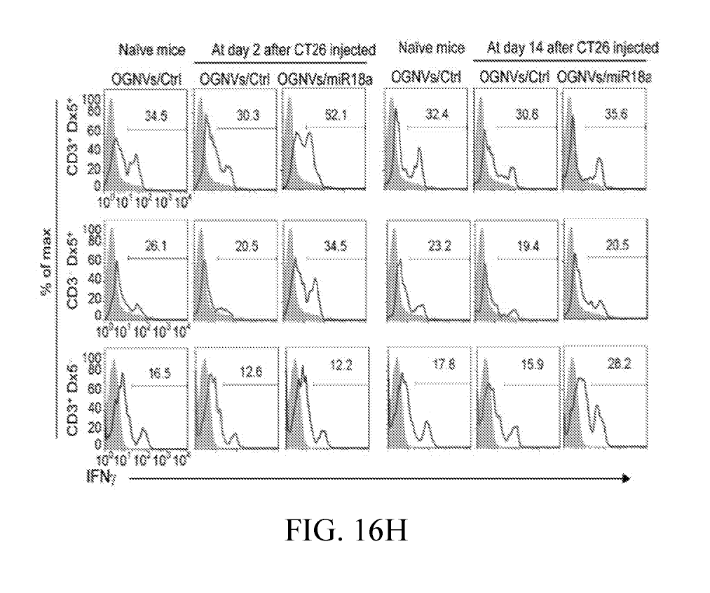

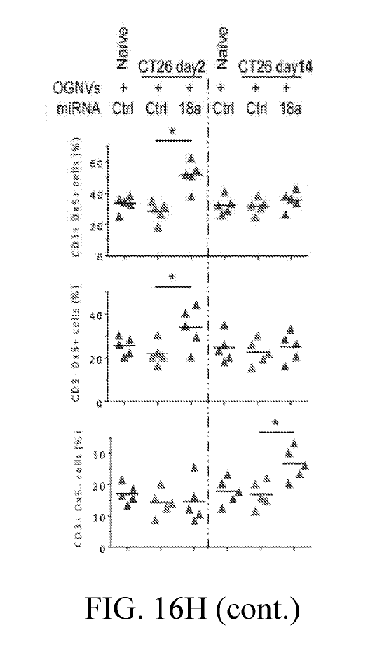

OGNV-miR18a treatment, as described in FIG. 16A, led to an increase in the percentages of F4/80⁺major histocompatibility complex (MHC)II⁺, F4/80⁺IL-12⁺ (M1), F4/80⁺interferon gamma (IFNγ)⁺ and F4/80⁺CD80⁺ cells (FIG. 16B). This increase is specific since the percentages of F4/80⁻CD86⁺ cells present in the liver of tumor bearing mice treated with OGNVs/Ctrl alone were no different from those treated with OGNVs-miR18a (FIG. 16B). It is well-known that M1 macrophages promote anti-tumor activity whereas M2 macrophages promote tumor progression. We further assessed the M1 versus M2 cytokine expressions in liver F4/80⁺ cells. miR18a treatment led to increasing percentages of F4/80⁺IFNγ⁺, F4/80⁺IL-12⁺, F4/80⁺CD80⁺, and decreasing percentages of F4/80⁺transforming growth factor beta (TGFβ)⁺, F4/80⁺CD206⁺ and F4/80⁺IL-10⁺ detected in the liver metastatic tumor bearing mice (FIG. 16B). This result was also supported by the data from quantitative analysis of the proteins expressed on FACS sorted F4/80 KCs (FIG. 16C). Consistent with flow cytometry results, OGNV-miR18a treatment dramatically increased the level of genes encoding IFNγ, IL-12, CD80, inducible nitric oxide synthase (iNOS), and decreased TGFβ expressed in F4/80 KCs isolated from metastatic liver (FIG. 16D). Collectively, miR18a treatment promoted induction of M1 macrophages (F4/80⁺IFNγ⁺ and F4/80⁺IL-12⁺) with upregulated co-stimulatory factors such as CD80, and iNOS while inhibiting M2 macrophages (F4/80⁺TGFβ, F4/80⁺IL-10⁺) in the liver of metastatic colon tumor bearing mice.

The inhibition of liver metastatic tumor growth in CT26 tumor bearing mice treated with OGNV-miR18a was also demonstrated. On day 14 after an intra-splenic injection of CT26 colon tumor cells, the number and size of tumor nodules in the liver of mice treated with vehicle were significantly increased in comparison with mice treated with OGNV-miR18a (FIG. 16E). This conclusion is also supported by the fact that there were fewer liver tumor foci, the liver weighed less in OGNV-miR18a treated mice (FIG. 16F) and these mice had a significantly prolonged survival (FIG. 16G).

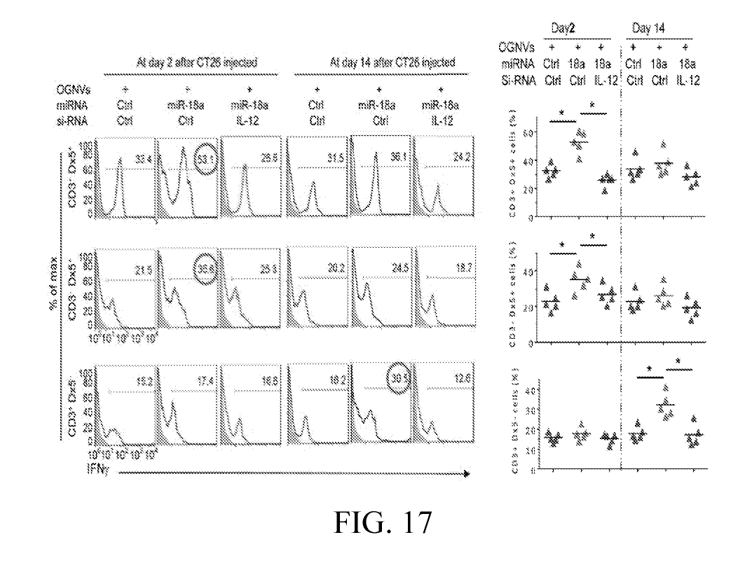

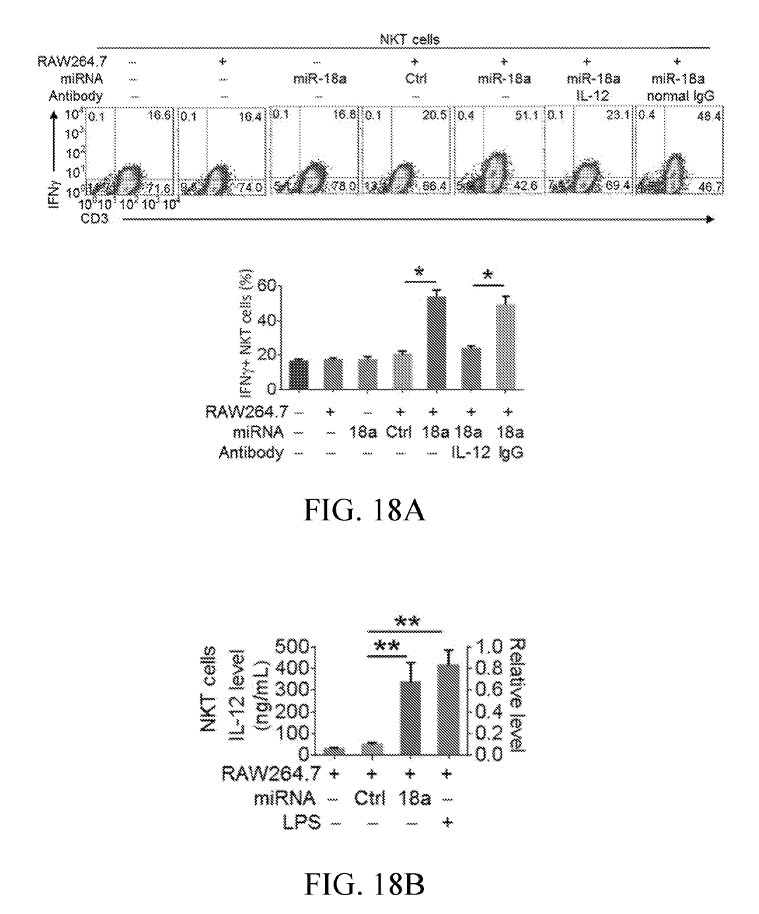

The induction of M1 macrophages promotes activation of NK, NKT and T cells. The data generated from FACS analysis indicated that at day 2 after OGNV-miR-18a treatment, both IFNγ⁺NKT (CD3⁺DX5⁺) and IFNγ⁻NK (CD3⁻DX5⁺) but not T(CD3⁺DX5⁻) cells were significantly induced; whereas, on day 14 induction of IFNγ⁺ CD3⁺T cells was dominant (FIG. 16H). To further demonstrate the role of macrophage-derived IL-12 induction of IFNγ⁺NK and IFNγ⁺NKT, mice treated with OGNVs co-encapsulating miR18a and IL-12 siRNA but not encapsulating IL-12 siRNA alone resulted in significant reduction of liver IFNγ⁺ NK and IFNγ+NKT, but had no effect on IFNγ⁺CD3⁺DX5⁻ T cells (FIG. 17). Consistent with in vivo results, neutralizing IL-12 in the supernatants of miR18a pre-transfected IL-12⁺RAW264.7 macrophage-like cells co-cultured with primary spleen NKT cells led to a significant reduction of IFNγ expressed in the NKT cells (FIGS. 18A-B). Collectively, these results suggest that F4/80⁺IL-12⁺ cells induced by OGNV-miR-18a plays a crucial role in the inhibition of liver metastasis of colon cancer.

### Example 7

### Liver Macrophages Play a Dominate Role in Inhibition of Colon Tumor Metastasis in the Liver

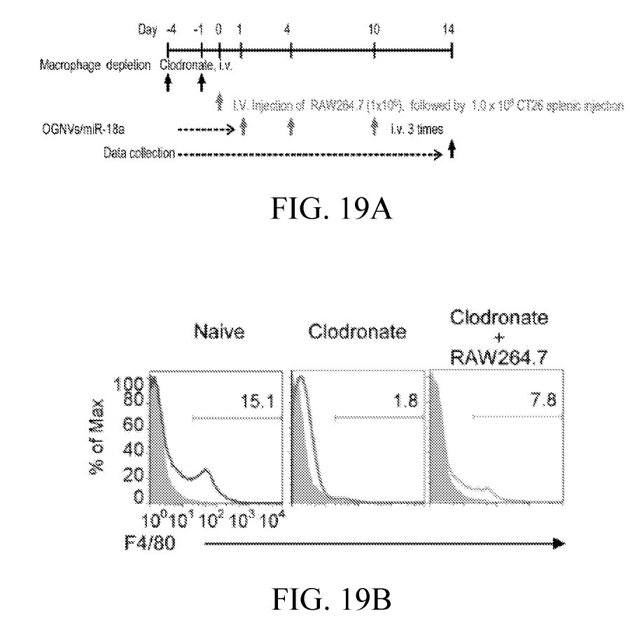

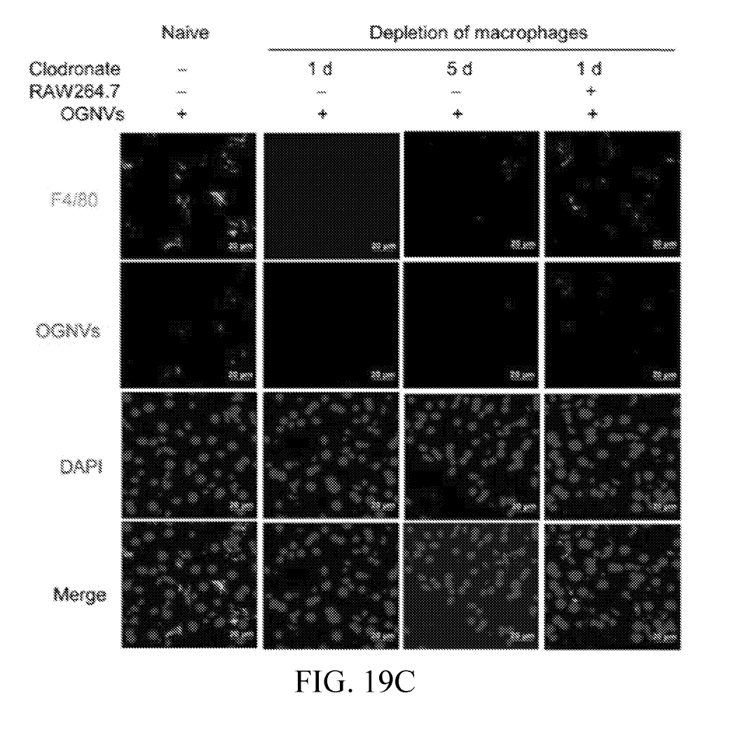

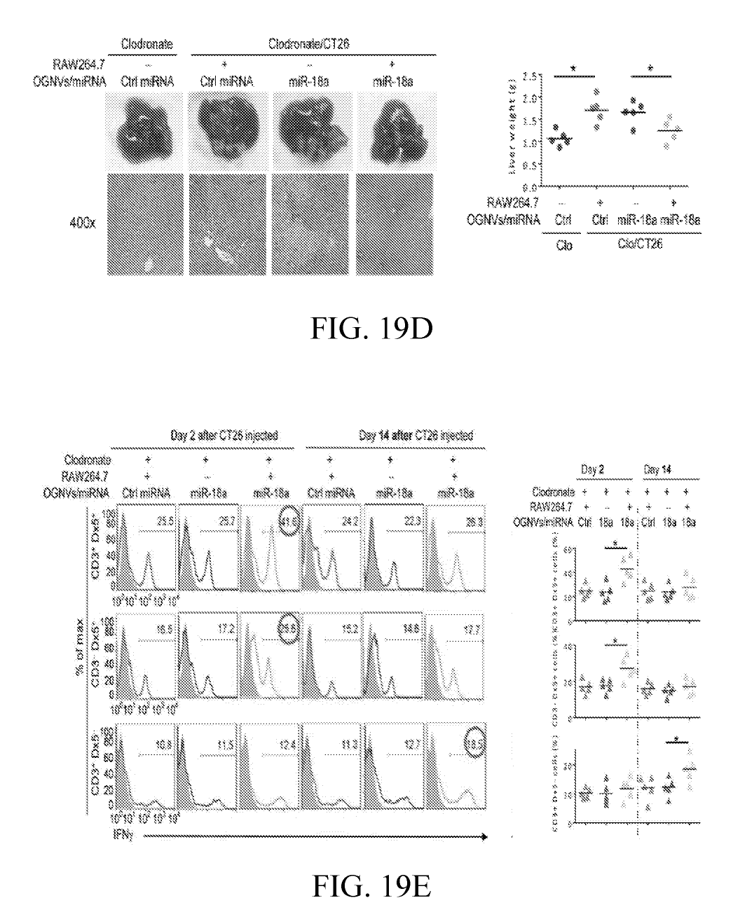

To identify whether the anti-tumor activity of miR-18a was directly mediated by liver macrophages, mice were repeatedly treated with clodronate liposome as described in FIG. 19A to deplete macrophages before an intra-splenic injection of CT26 cells. Depletion of macrophages (FIG. 19B-C) abolished the anti-tumor activity of miR-18a, and the miR18a-mediated anti-tumor activity was restored by adoptive transfer of macrophage-like RAW264.7 cells (FIG. 19D). This conclusion is also supported by the significant induction of liver IFNγ⁺NKT and IFNγ⁺NK cells at day 2 and IFNγ⁺CD3⁺T cells on day 14 after RAW264.7 cells were adoptively transferred into macrophage depleted mice (FIG. 19E).

### Example 8

### miR18a-mediated Inhibition of the Growth of Liver Metastasis of Colon Tumor Cells is IFNγ Dependent

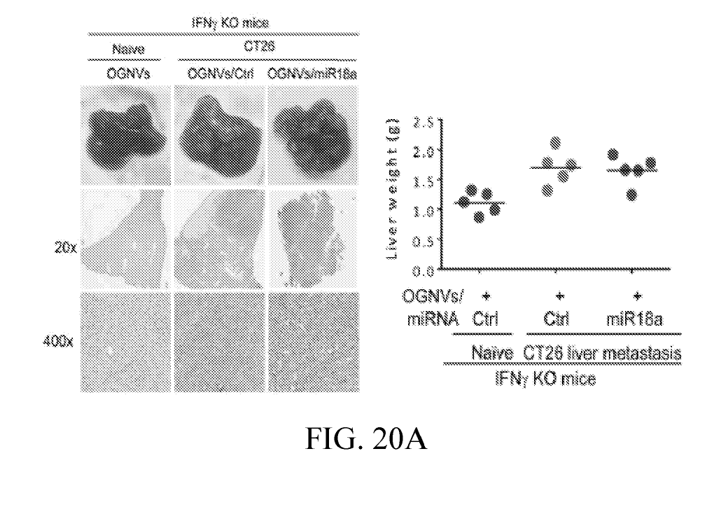

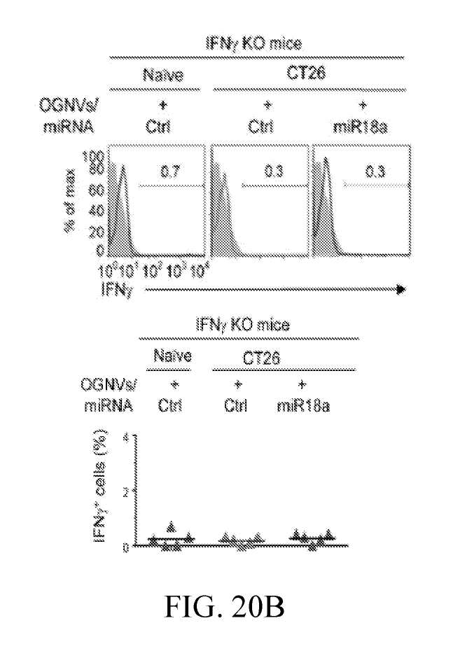

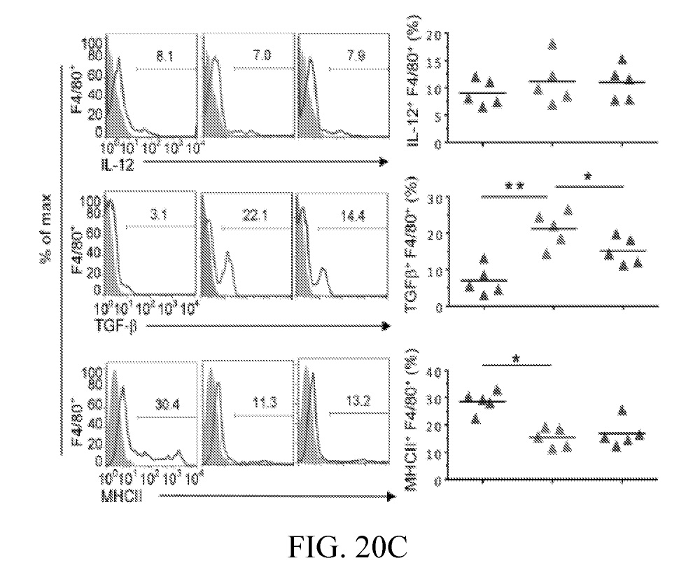

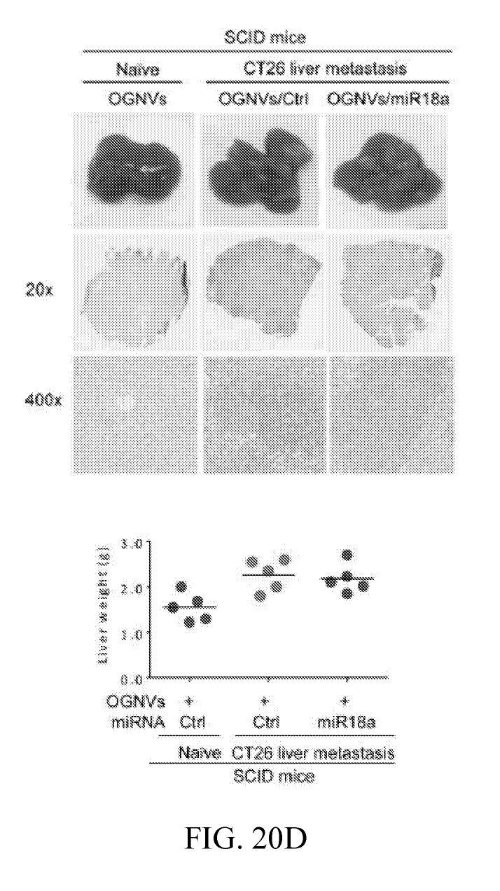

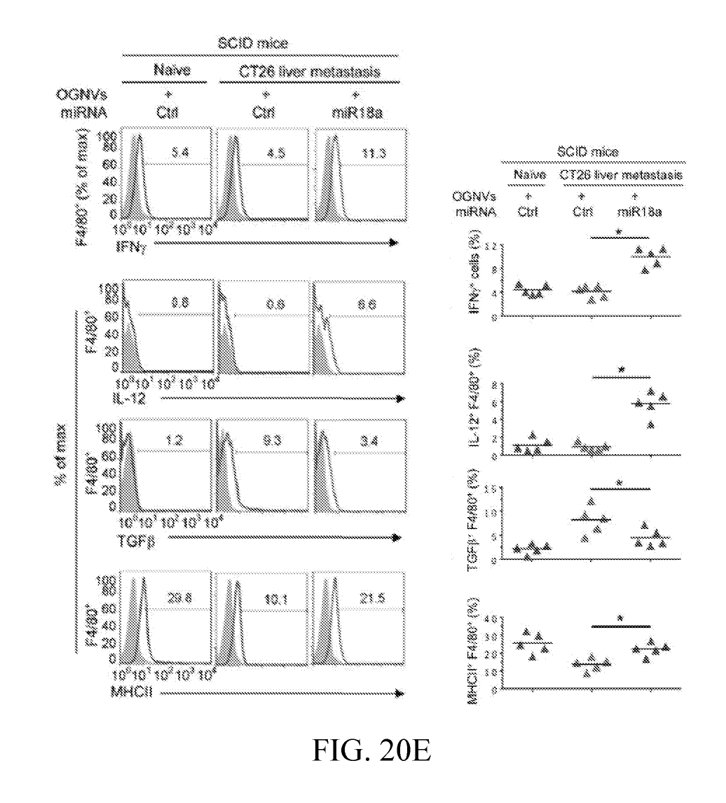

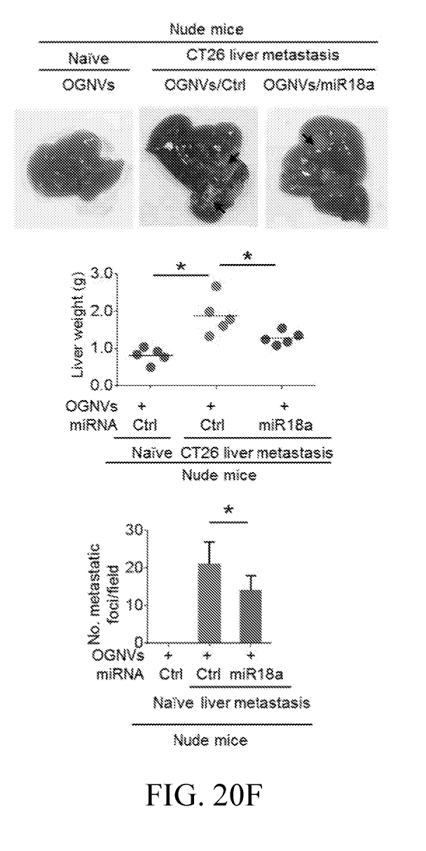

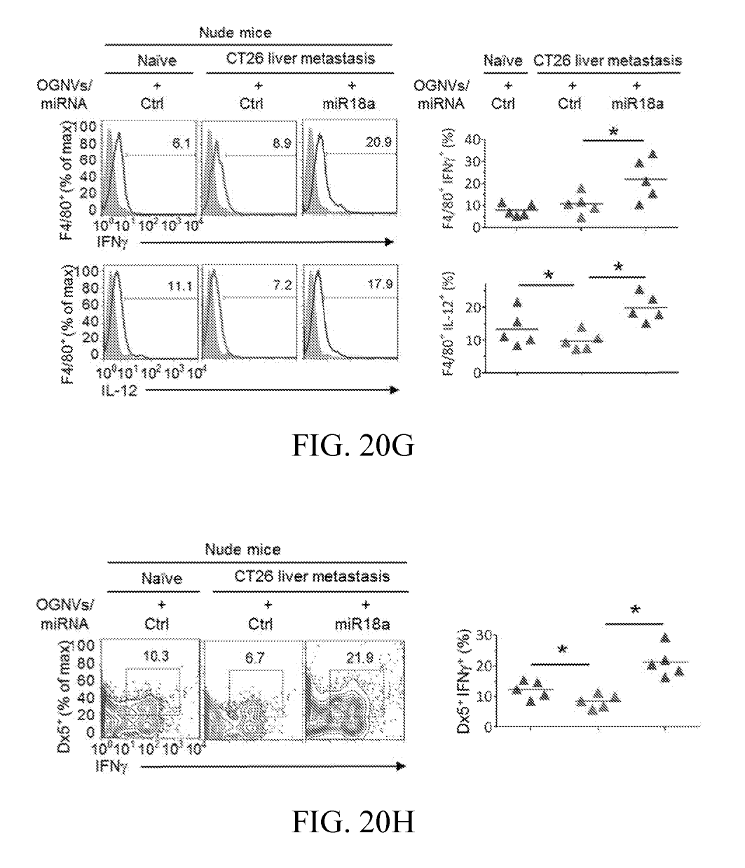

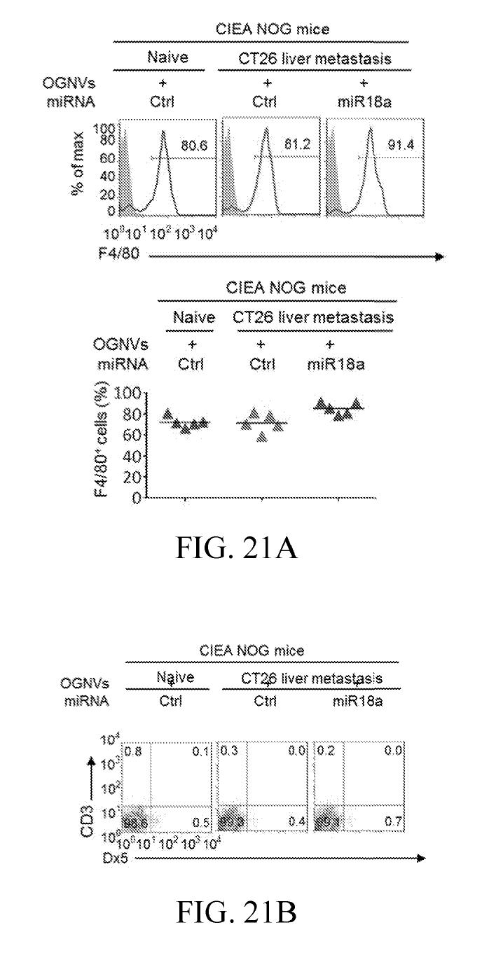

To determine whether the effect of miR18a against liver metastasis of colon cancer results from induction of KC IFNγ, CT26 colon carcinoma cells were intra-splenic injected into IFNγ knock out (KO) mice. On day 14 after tumor cell inoculation, OGNVs/miR18a treatment showed no evidence of inhibiting tumor growth in IFNγ KO mice. Mice treated with OGNVs/control (Ctrl)-miRNA alone and OGNVs/miR18a were similar in liver size and weight (FIG. 20A). The H&E stained sections of liver from both groups displayed similar pathology of liver metastasis (FIG. 20A). As expected, IFNγ expression was not found on leukocytes or F4/80 cells from the livers in IFNγ KO mice (FIG. 20B). Evidence for the effect of miR-18a on induction of F4/80⁺IL-12⁺was not obtained in IFNγ KO mice although the expression of TGFβ was still repressed by miR-18a (FIG. 20C). Collectively, these results indicate that KC IFNγ is an upstream cytokine of IL12 for miR-18a mediated induction of M1 macrophages. KC IFNγ is required for miR18a-mediated induction of IL-12. Induction of macrophage IL-12 further enhances activation of NK and NKT cells at positive feed-back manner. To further clarify the role of NK, NKT and T cells on the inhibition of tumor metastasis caused by miR-18a, NOG mice which are deficient for NK, NKT, and T cells were challenged with CT26 tumor cells using the identical protocol described for induction of liver metastasis of colon cancer in a wild-type BALB/c mouse model (FIG. 16). As expected, multi-administration of OGNVs-miR-18a did not lead to inhibition of tumor metastasis in the NOG mice (FIG. 20D) although F4/80⁺IFNγ⁺, F4/80⁺IL-12⁺and F4/80⁺MHCII⁺ cells (FIG. 20E) were still induced. The fact that the frequency of CD3⁻and Dx5⁺cells were undetectable in naïve or tumor bearing NOG mice (FIGS. 21A-B) regardless of treatment supports the idea that NK, NKT, or T cells are effector cells responsible for inhibition of liver metastasis of colon cancer cells. In contrast, the data generated from nude mice (FIG. 20F) which have both NK and NKT cell activity suggest that NK and NKT cells play a critical role in the inhibition of tumor metastasis caused by miR-18a. The effects of miR-18a on induction on IFNγ⁺IL-12⁺KCs (FIG. 20G) and IFNγ⁺NK⁺ cells (FIG. 20H) has no impact in T cell deficient nude mice. In combination with data generated from macrophage depletion, IFNγ KO mice and NOG and nude mice, these data suggest that miR18a delivered by OGNVs initially induces expression of IFNγ in macrophages, which is required for induction of macrophage IL-12. Subsequently, macrophage IL-12 amplifies the miR18a-mediated anti-tumor activity by activation of liver NK and NKT cells in an IFNγ dependent manner.

### Example 9

### miR-18a Suppresses Liver Metastasis of Colon Cancer Triggered by Directly Targeting IRF2

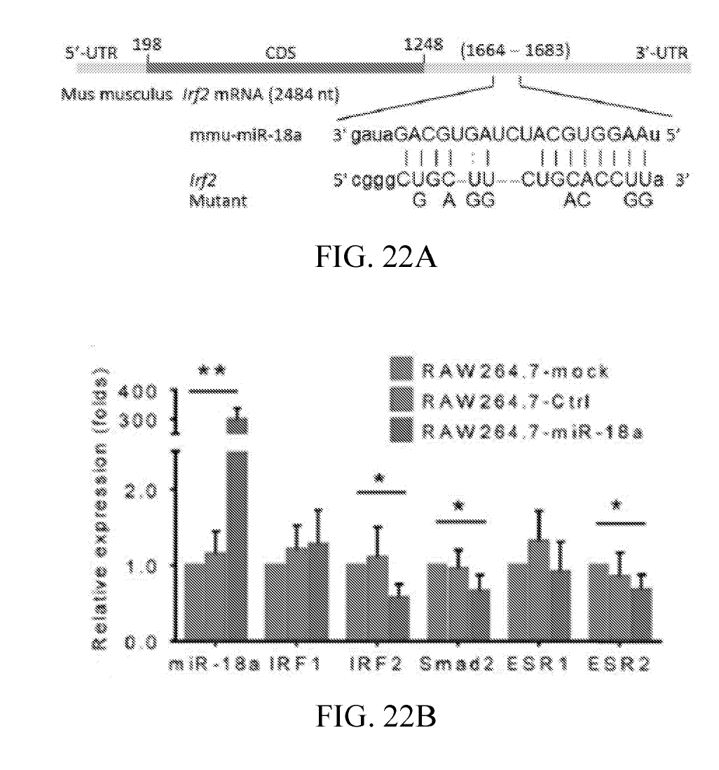

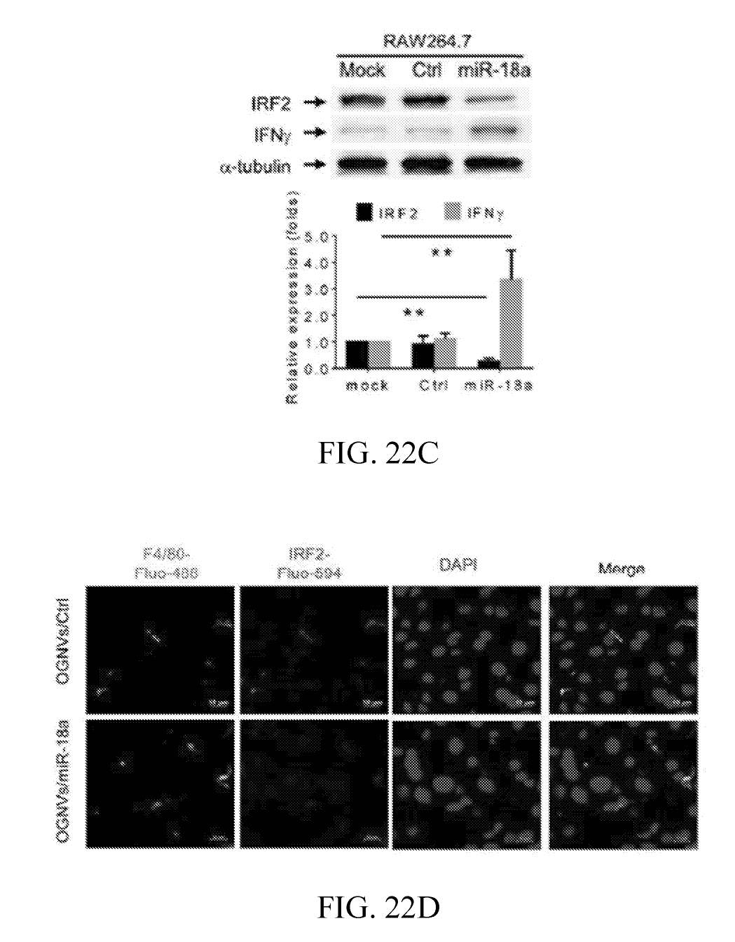

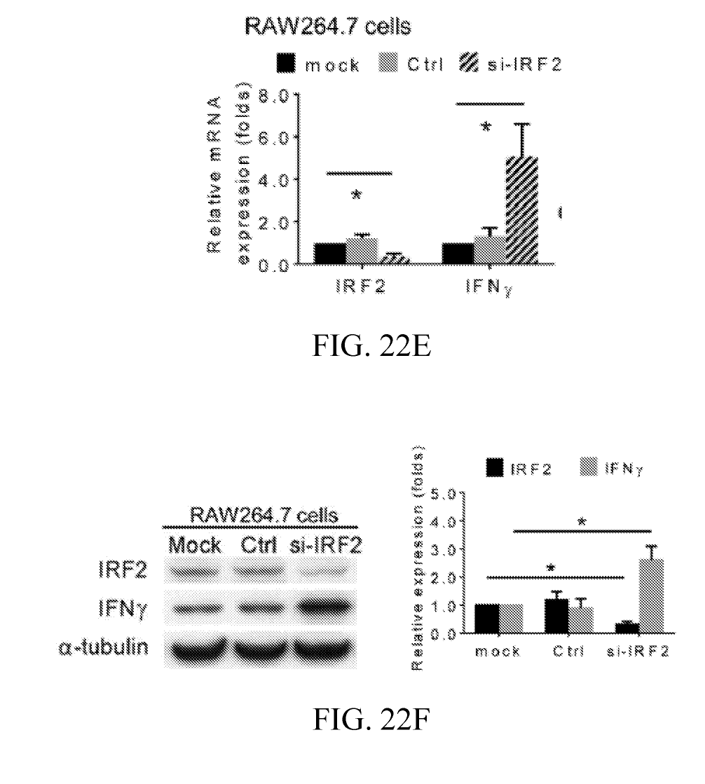

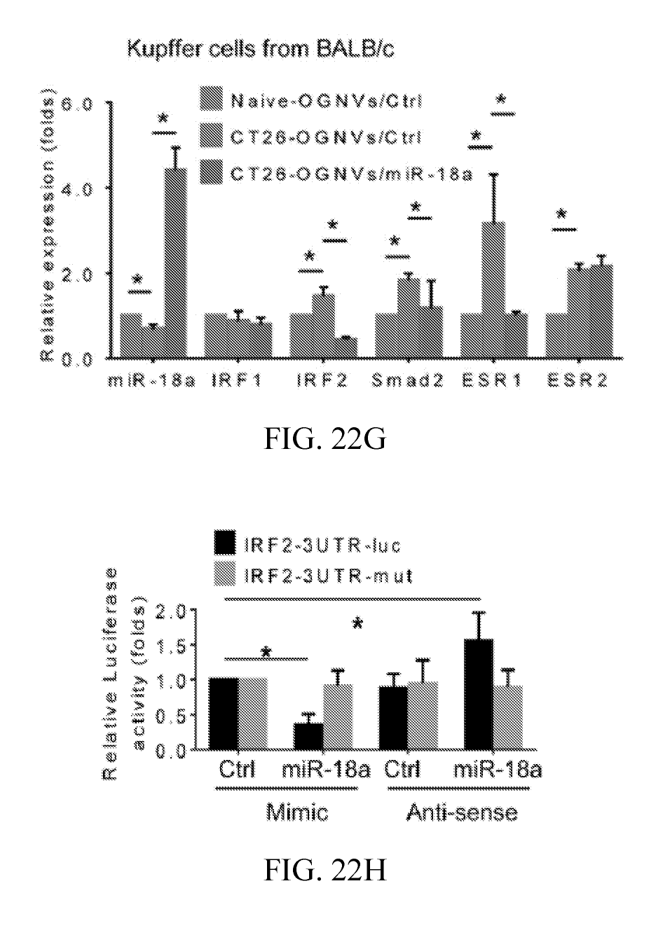

Given the profound anti-colon tumor metastasis effect of miR-18a delivered by OGNVs, how miR-18a induces the expression of IFNγ in macrophages required further investigated. We first searched miRNA databases for potential miR-18a targets that may possibly contribute to IFNγ induction. The three public miRNA databases (TargetScan, Pictar, and MicroRNA) all predicted that Irf2 might be a target for miR-18a; the 3′-UTR of Irf2 contains a highly conserved binding site from position 1668 to 1682 for miR-18a (FIG. 22A). To determine whether miR-18a could target Irf2 in macrophage cells, we transfected the mouse mature miR-18a mimic into BALB/c-derived macrophage-like RAW264.7 cells. The RAW264.7 cells transfected with OGNVs/miR18a have significantly down-regulated IRF2 mRNA expression (FIG. 22B) as well as IRF2 protein expression (FIG. 22C-D). We also found IFNγinduction by OGNVs/miR18a following reduction of Irf2 (FIG. 22C). Irf2 siRNA repressed Irf2 expression in RAW264.7 cells and led to increasing IFNγexpression (FIG. 22E-F). These in vitro results were further confirmed in the liver KCs isolated from liver metastasis in CT26 mice administrated OGNVs/miR18a (FIG. 22G). To ascertain the direct effect of miR-18a on Irf2, a mutant construct that would disrupt the predicted miR-18a binding site was generated from pEZX-MT01- Irf2 containing a 1,234 bp length 3′UTR of Irf2 mRNA (Gene Accession: NM_008391.4). We performed a luciferase reporter assay by co-transfecting a vector containing IRF2 3′UTR fused luciferase and miR-18a or control miRNA as a negative control. Overexpression of miR-18a decreased the luciferase activity of the reporter with 3′UTR of Irf2 by about 60% in RAW264.7 cells (FIG. 22H). However, mutation that disrupted the binding site for miR-18a entirely restored luciferase activity. Moreover, overexpression of anti-sense (AS) miR-18a caused induction of luciferase and no inductive effect of AS-miR-18a on the activity of the reporter when a mutant 3′UTR of Irf2 was detected. These results demonstrate that Irf2 is a target of miR-18a in macrophages.

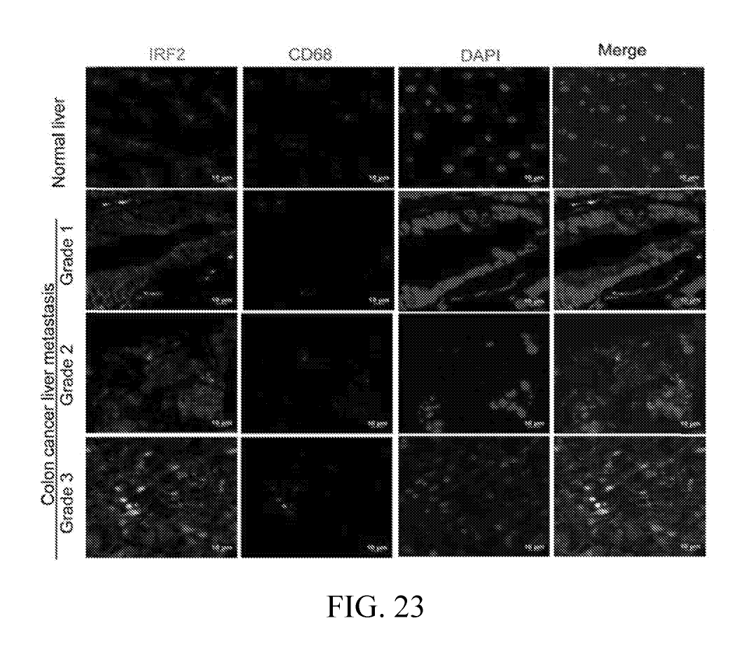

We further determined whether the Irf2 was up-regulated in the metastatic liver tissue of colon cancer patients. The results from immunohistological staining of CD68 and IRF2 in human liver sections (FIG. 23) suggest that IRF2 is expressed in liver CD68 macrophages. More importantly, the levels of expression of IRF2 in the liver of human colon metastatic patients are increased as the disease progresses. These results indicated that IRF2 expression correlates with liver metastasis differentiation in colorectal cancer.

Discussion of Examples 5-9

Metastasis accounts for the majority of cancer deaths. The liver is a frequent site of metastasis of many different types of cancer, including those of the gastrointestinal tract, colon, breast, lung, and pancreas. Most treatments are not effective for liver metastasis because liver metastases represent cancer that has spread from another part of the body. We hypothesize that boosting the strength of anti-tumor immune responses may be a better way to treat liver metastasis; in particular, creating a liver microenvironment that is dominated by anti-tumor M1 macrophages.

Liver macrophages (Kupffer cells; KCs) play a crucial role in the pathogenesis of liver tumor metastasis and are a major component of the microenvironment of primary and metastatic liver tumors. Direct and indirect activation of KCs results in the production of factors and cytokines capable of facilitating both anti-tumor and pro-tumor effects. More importantly, Kupffer cells are situated in the hepatic sinusoids to encounter circulating T cells, as well as natural killer (NK) and natural killer T (NKT) cells, and modulate activity of these lymphocytes. Interaction with these immune cell populations is required to develop the full potential of KCs to mediate anti-tumor immunity. Therefore, targeted delivery of therapeutic agents to liver KCs could enhance anti-tumor immune functions.

Evidence is provided that liver macrophages can make M1 or M2 responses. M1 and M2 macrophages promote Th1 and Th2 responses, respectively. M2 macrophages are a major component of the leukocyte infiltrate of tumors. M2 macrophages suppress NK, NKT, and T-cell activation and proliferation by releasing transforming growth factor beta (TGF-β). Moreover, they have an interleukin (IL)-12 low phenotype, characteristic of M2 cells. By expressing properties of polarized M2 cells, M2 participate in circuits that regulate tumor growth and progression, adaptive immunity, stroma formation and angiogenesis. This raises the possibility that the molecules and cells involved might represent novel and valuable therapeutic targets. As for M1 macrophages, these macrophages produce IL-12 to promote tumoricidal responses. The mechanisms governing macrophage polarization are unclear.

MicroRNAs (miRNAs) are a class of small, non-coding RNAs that post-transcriptionally control the translation and stability of mRNAs. Hundreds of miRNAs are known to have dysregulated expression in cancer. Studies evaluating their biological and molecular roles and their potential therapeutic applications are emerging. The levels of miRNAs expressed in myeloid cells have effects on the polarization of M1 versus M2 macrophages. Targeted delivery of miRNAs to macrophages as an alternative strategy for treatment of cancer by induction of M1 macrophages has not been fully developed.

MiR-18a, an important member of miR-17-92 family, has been shown various effects on different tumors. It was reported that miR-18a could act as a tumor suppressor. Our previous study published showed that miR-18a suppresses colon tumor growth by targeting β-catenin expressed in the colon tumor cells. The effects of miR-18a on the polarization of M1 versus M2 macrophages have not been reported. We attempted to predict the potential target genes of miR-18a through applying a bioinformatics analysis method (TargetScan). We found Irf2, a theoretical target gene of miR-18a with the specific binding site in the 3′-UTR sequence. IL-12 is dysregulated in macrophages from Irf2 knockout mice. This finding led us to choose miR18a as an example to test whether a grapefruit-derived nanovector (GNV) based delivery system can be used for targeted delivery of therapeutic miRNA to liver macrophages and treat liver metastasis.

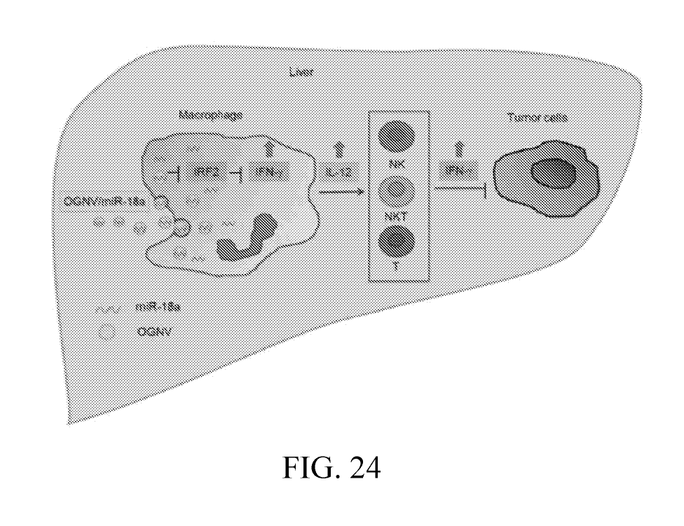

In this study, our main finding is highlighted in a novel regulatory mechanism of M1 macrophage functioning along the IFN-γ/Irf2 axis mediated by miR-18a (FIG. 24). These findings establish a proof of concept and the basis for treating liver metastasis of colon cancer by mediating macrophage populations which in turn could be applicable to other types of cancers and macrophage-mediated inflammatory diseases.

Liver macrophages are not only pleiotropic cells that can function as immune effectors and regulators, tissue remodelers, or scavengers, but also have unique location. KCs are stationary cells located in the vasculature, adherent to liver sinusoidal endothelial cells (LSECs) and directly exposed to the contents of blood. This is in contrast to other monocyte and macrophage cell populations located in other tissues that actively crawl through the tissue in search of pathogens or nano/micro particles. Importantly, the size of most nanoparticles, including GNVs, makes them favorable to being trapped in the liver. In addition, KCs represent 80-90% of all tissue macrophages in the entire body. Collectively, these KCs features made GNVs favorable homing to the liver. The data presented in this study suggest that liver macrophages are preferentially targeted by GNV, and miR18a delivered by GNVs to promote liver anti-tumor M1 macrophages induction. Since the liver is one of the major organs involved in metastasis for a number of different types of cancers, including colon cancer, and M1 macrophages play a role in an anti-tumor progression in general, our strategy could also be applied to treat other types of cancer with liver metastasis.

The acute inflammatory response is characterized by the presence of liver M1 macrophages, and the chronic or resolution of inflammatory phases is mediated by the enrichment of M2 macrophages. M1 macrophages are known to enhance anti-tumor growth and microbial clearance, and M2 macrophages are known to enhance liver tissue repair and to secrete pro-resolution substances including TGF-β. Therefore, targeted delivery of specific therapeutic agents which can modulate polarization of liver macrophages is critical. Our data presented in this study indicate that OGNVs are taken up by liver macrophages. The data we recently published and present in this study suggest that unlike commercially available vectors, OGNVs are non-toxic to the macrophages and liver and can be easily produced on a large scale basis for clinical applications and are capable of delivering a variety of different types of therapeutic agents.

In this study, we further optimized the conditions for OGNV delivery of mRNAs and miRNAs. Therefore, without manipulation of the OGNV, such as adding a targeting moiety, therapeutic agents delivered by OGNVs automatically get into liver macrophages with no toxic effects.

Different microRNAs are expressed in M1 or M2 macrophages and have been shown to control macrophage polarization. The role of miR-18a in macrophage polarization is unknown but immunomodulation of dendritic cell function of miR18a has been described. We found that liver macrophages are polarized to M1 macrophages after miR18a is delivered by OGNVs. The molecular mechanisms involved in miR-18a -induced M1 macrophages were further studied and we found that miR18a-mediated induction of macrophage IFNγ is required for inhibition of liver metastasis of colon cancer and that macrophage IRF2 is targeted by miR18a.

Unlike the situation with artificially synthesized nanoparticles, recently, we have developed grapefruit-derived nanovectors (GNVs) which can deliver a variety of therapeutic agents including chemotherapeutic compounds, DNA expression vectors, siRNA and proteins such as antibodies. GNVs have a number of advantages over other delivery systems, including low toxicity, large scale production with low cost, and easily biodegradable without biohazards to the environment. However optimization of GNVs to maximize carrying therapeutic agents has not been studied. In this study, using miR18a as an example, we found that optimized GNVs (OGNVs) are capable of encapsulating miR18a and the ability was significantly increased by short pre-exposure of the GNVs mixed with miR18a buffered with an optimized concentration of Na+with exposure to ultraviolet (UV) light. We further demonstrate that miR-18a delivered by GNVs inhibits the growth of colon tumors that have metastasized to the liver by polarizing KCs to M1 cells (F4/80⁺IFNγ⁺IL-12⁺). miR18a mediated induction of M1 IFNγ⁺is required for production of IL-12. IL-12 subsequently triggers the activation of liver immune cells including NK and NKT cells. NOG mice lack mature T cells and functional NK cells. This role of IL-12 was also supported in NOG mice injected with CT26 colon tumor cells by the fact that miR-18a delivered by GNVs does not inhibit the growth of colon tumors that have metastasized to the liver. Nude mice which have both NK and NKT activity were found to inhibit the growth of metastasized tumors in the liver when injected with CT26 colon tumor cells. Although IL-12 has been shown to enhance the rejection of a variety of murine tumors, pre-clinical and clinical studies have revealed that IL-12 can produce severe toxicity[44]. Therefore, our finding that induction of IL-12 through KC IFN-γ induced through the GNVmiR18a axis in the liver will have less side-effects compared to systemic administration IL-12 has great potential for anti-cancer immune therapy.

This study addresses the question of not only mechanisms that regulate the induction of M1 macrophages but also the use of grapefruit-derived nanovectors (GNVs) as a therapeutic vehicle for treatment of liver metastasis of colon cancer. We identified miR18a as a previously unrecognized inhibitor for liver metastasis through the induction of M1 macrophage. These results provide new insights into the molecular mechanisms of miR18a-mediated macrophage polarization and shed light on new therapies for cancers through a miR18a-mediated induction of M1 macrophages. The means and method we demonstrated in this study are a major step in the development of high capacity GNVs to deliver therapeutic RNA in general.

Our findings established a basis for further investigating whether IRF2 acts as a suppressor to directly inhibit expression of IFNγ. Alternatively, it is possible that as a result of miR18a-mediated down regulation of levels of IRF2, the level of IRF1 is increased. An imbalance between IRF-1 and IRF-2 (43, 44), the activator and repressor of IFN responses, respectively, may contribute to the altered expression of IFNγ. Therefore, increasing IRF-1/IRF-2 ratios by targeted delivery of miR18a to IRF2 overexpressed macrophages is expected to induce IFNγ.

Systemic delivery of targeted vectors presents major challenges for developing an effective anti-cancer immunotherapy. One of advantages of an OGNV based delivering system is that OGNV is selectively taken up by liver KCs, not hepatocytes. Targeted delivery is particularly important for miRNA mediated therapy. One miRNA could regulate a number of genes, and among the potentially targeted genes, preferential miRNA targeted genes may be dependent on the levels of that miRNA and the accessibility and availability of the miRNA targeted genes. It is conceivable that the mRNA expression profile of one type of cell, such as KCs, targeted by OGNVs could be different from the hepatocytes. Therefore, genes targeted by miR18a in KCs are unlikely the same ones if miR18a is overexpressed in other types of cells such as hepatocytes. It has been reported that over expression of miR18a in hepatocytes may contribute to the pathogenicity of liver cancer. Our real-time PCR data showed that the level of miR18a in hepatocytes was not increased following an intravenous administration of OGNVs/miR-18a. This could be due to OGNVs/miR-18a primarily being taken up by KCs. The exploitation of the liver macrophages to mediate the immune therapeutic effects of miRNA, such as miR-18a delivered by GNVs, can circumvent limitations of miRNA targeted delivery. Kupffer cells are the first point of contact to administer miRNAs encapsulated in OGNVs, affording an opportunity to directly modulate their functional activity. Therefore, besides of miRNAs, an OGNV based in vivo delivery system can also deliver other therapeutic agents which modulate liver macrophage activity and control macrophage lineage. OGNVs based targeting liver macrophage naturally take place without pressure on the host. Therefore, we do not expect that GNV based targeted delivery to KCs would be altered due host pressure built up as other delivery system.

In summary, the Examples above provide evidence for the role of miR18a in the induction of liver M1 (F4/80⁺interferon gamma (IFNγ)⁺IL-12⁺) macrophages. The Examples show that miR18a encapsulated in grapefruit-derived nanovector (GNV) mediated inhibition of liver metastasis that is dependent upon the induction of M1 (F4/80⁺IFNγ⁺IL-12⁺) macrophages; depletion of macrophages eliminated its anti-metastasis effect. Furthermore, the miR18a mediated induction of macrophage IFNγ by targeting IRF2 is required for subsequent induction of IL-12. IL-12 then activates natural killer (NK) and natural killer T (NKT) cells for inhibition of liver metastasis of colon cancer. This conclusion is supported by the fact that knockout of IFNγ eliminates miR18a mediated induction of IL-12, miR18 treatment has an anti-metastatic effects in T cell deficient mice but there is no anti-metastatic effect on NK and NKT deficient mice. Co-delivery of miR18a and siRNA IL-12 to macrophages did not result in activation of co-cultured NK and NKT cells. Taken together these results indicate that miR18a can act as an inhibitor for liver metastasis through induction of M1 macrophages.

Statistical Analysis

Statistical significance was determined by the Student's t test. Differences between individual groups were analyzed by one- or two-way analysis of variance test. Differences were considered significantly when the P value was less than 0.05 or 0.01 as indicated in the text.

Throughout this document, various references are mentioned. All such references are incorporated herein by reference, including the references set forth in the following list:

### REFERENCES

1. Ong, W Y, Shalini, S M, and Costantino, L (2014). Nose-to-brain drug delivery by nanoparticles in the treatment of neurological disorders. Current medicinal chemistry 21: 4247-4256.
2. Kozlovskaya, L, Abou-Kaoud, M, and Stepensky, D (2014). Quantitative analysis of drug delivery to the brain via nasal route. Journal of controlled release : official journal of the Controlled Release Society 189: 133-140.
3. Larsen, J M, Martin, D R, and Byrne, M E (2014). Recent advances in delivery through the blood-brain barrier. Current topics in medicinal chemistry 14: 1148-1160.
4. Zhuang, X, Xiang, X, Grizzle, W, Sun, D, Zhang, S, Axtell, R C, et al. (2011). Treatment of brain inflammatory diseases by delivering exosome encapsulated anti-inflammatory drugs from the nasal region to the brain. Molecular therapy : the journal of the American Society of Gene Therapy 19: 1769-1779.
5. Wang, Q, Ren, Y, Mu, J, Egilmez, N K, Zhuang, X, Deng, Z, et al. (2015). Grapefruit-derived nanovectors use an activated leukocyte trafficking pathway to deliver therapeutic agents to inflammatory tumor sites. Cancer research.
6. Wang, Q, Zhuang, X, Mu, J, Deng, ZB, Jiang, H, Zhang, L, et al. (2013). Delivery of therapeutic agents by nanoparticles made of grapefruit-derived lipids. Nature communications 4: 1867.
7. Islam, M A, Park, T E, Singh, B, Maharj an, S, Firdous, J, Cho, M H, et al. (2014). Major degradable polycations as carriers for DNA and siRNA. Journal of controlled release : official journal of the Controlled Release Society 193: 74-89.
8. Buzea, C, Pacheco, II, and Robbie, K (2007). Nanomaterials and nanoparticles: sources and toxicity. Biointerphases 2: MR17-71.
9. Patnaik, S, and Gupta, K C (2013). Novel polyethylenimine-derived nanoparticles for in vivo gene delivery. Expert opinion on drug delivery 10: 215-228.
10. Chaudhury, A, and Das, S (2015). Folate receptor targeted liposomes encapsulating anti-cancer drugs. Current pharmaceutical biotechnology 16: 333-343.
11. Marchetti, C, Palaia, I, Giorgini, M, De Medici, C, Iadarola, R, Vertechy, L, et al. (2014). Targeted drug delivery via folate receptors in recurrent ovarian cancer: a review. OncoTargets and therapy 7: 1223-1236.
12. Serpe, L, Gallicchio, M, Canaparo, R, and Dosio, F (2014). Targeted treatment of folate receptor-positive platinum-resistant ovarian cancer and companion diagnostics, with specific focus on vintafolide and etarfolatide. Pharmacogenomics and personalized medicine 7: 31-42.
13. Muller, C (2012). Folate based radiopharmaceuticals for imaging and therapy of cancer and inflammation. Current pharmaceutical design 18: 1058-1083.
14. Franzen, S (2011). A comparison of peptide and folate receptor targeting of cancer cells: from single agent to nanoparticle. Expert opinion on drug delivery 8: 281-298.
15. Dhawan, D, Ramos-Vara, J A, Naughton, J F, Cheng, L, Low, P S, Rothenbuhler, R, et al. (2013). Targeting folate receptors to treat invasive urinary bladder cancer. Cancer research 73: 875-884.
16. Leamon, C P, Reddy, J A, Vetzel, M, Dorton, R, Westrick, E, Parker, N, et al. (2008). Folate targeting enables durable and specific antitumor responses from a therapeutically null tubulysin B analogue. Cancer research 68: 9839-9844.
17. Jiang, H, Wang, P, Li, X, Wang, Q, Deng, Z B, Zhuang, X, et al. (2014). Restoration of miR17/20a in solid tumor cells enhances the natural killer cell antitumor activity by targeting Mekk2. Cancer immunology research 2: 789-799.
18. Kularatne, S A, and Low, P S (2010). Targeting of nanoparticles: folate receptor. Methods in molecular biology 624: 249-265.
19. Zhao, X B, Muthusamy, N, Byrd, J C, and Lee, R J (2007). Cholesterol as a bilayer anchor for PEGylation and targeting ligand in folate-receptor-targeted liposomes. Journal of pharmaceutical sciences 96: 2424-2435.
20. Dauty, E, Remy, J S, Zuber, G, and Behr, J P (2002). Intracellular delivery of nanometric DNA particles via the folate receptor. Bioconjugate chemistry 13: 831-839.
21. Wang, H, Zheng, X, Behm, F G, and Ratnam, M (2000). Differentiation-independent retinoid induction of folate receptor type beta, a potential tumor target in myeloid leukemia. Blood 96: 3529-3536.
22. Reddy, J A, Dean, D, Kennedy, M D, and Low, P S (1999). Optimization of folate-conjugated liposomal vectors for folate receptor-mediated gene therapy. Journal of pharmaceutical sciences 88: 1112-1118.
23. Mayor, S, Sabharanjak, S, and Maxfield, F R (1998). Cholesterol-dependent retention of GPI-anchored proteins in endosomes. The EMBO journal 17: 4626-4638.
24. Ding, L, Liu, Z, Aggrey, M O, Li, C, Chen, J, and Tong, L (2015). Nanotoxicity: the toxicity research progress of metal and metal- containing nanoparticles. Mini reviews in medicinal chemistry 15: 529-542.
25. Czajka, M, Sawicki, K, Sikorska, K, Popek, S, Kruszewski, M, and Kapka-Skrzypczak, L (2015). Toxicity of titanium dioxide nanoparticles in central nervous system. Toxicology in vitro : an international journal published in association with BIBRA 29: 1042-1052.
26. Zoroddu, M A, Medici, S, Ledda, A, Nurchi, V M, Lachowicz, J I, and Peana, M (2014). Toxicity of nanoparticles. Current medicinal chemistry 21: 3837-3853.
27. Nafisi, S, Schafer-Korting, M, and Maibach, HI (2014). Perspectives on percutaneous penetration: Silica nanoparticles. Nanotoxicology: 1-15.
28. Bligh, E G, and Dyer, W J (1959). A rapid method of total lipid extraction and purification. Canadian journal of biochemistry and physiology 37: 911-917.
29. Rouser, G, Siakotos, A N, and Fleischer, S (1966). Quantitative analysis of phospholipids by thin-layer chromatography and phosphorus analysis of spots. Lipids 1: 85-86.
30. Ungaro, F, De Rosa, G, Miro, A, and Quaglia, F (2003). Spectrophotometric determination of polyethylenimine in the presence of an oligonucleotide for the characterization of controlled release formulations. Journal of pharmaceutical and biomedical analysis 31: 143-149.
31. Youniss, F M, Sundaresan, G, Graham, L J, Wang, L, Berry, C R, Dewkar, G K, et al. (2014). Near-infrared imaging of adoptive immune cell therapy in breast cancer model using cell membrane labeling. PloS one 9: e109162.
32. Ezzat, T, Dhar, D K, Malago, M, and Olde Damink, S W (2012). Dynamic tracking of stem cells in an acute liver failure model. World journal of gastroenterology : WJG 18: 507-516.
33. Cho, H, Indig, G L, Weichert, J, Shin, H C, and Kwon, G S (2012). In vivo cancer imaging by poly(ethylene glycol)-b-poly(varepsilon-caprolactone) micelles containing a near-infrared probe. Nanomedicine : nanotechnology, biology, and medicine 8: 228-236.
34. Shan, L (2004). Near-infrared fluorescence 1,1-dioctadecyl-3,3,3,3-tetramethylindotricarbocyanine iodide (DiR)-labeled macrophages for cell imaging. Molecular Imaging and Contrast Agent Database (MICAD): Bethesda (Md.).
35. Lujambio A, Akkari L, Simon J, Grace D, Tschaharganeh DF, Bolden J E, Zhao Z, Thapar V, Joyce J A, Krizhanovsky V and Lowe S W. Non-cell-autonomous tumor suppression by p53. Cell. 2013; 153(2):449-460.
36. Rushfeldt C, Sveinbjornsson B, Seljelid R and Smedsrod B. Early events of hepatic metastasis formation in mice: role of Kupffer and NK-cells in natural and interferon-gamma-stimulated defense. The Journal of surgical research. 1999; 82(2):209-215.
37. Shiratori Y, Nakata R, Okano K, Komatsu Y, Shiina S, Kawase T, Sugimoto T, Omata M and Tanaka M. Inhibition of hepatic metastasis of colon carcinoma by asialo GM1-positive cells in the liver. Hepatology. 1992; 16(2):469-478.
38. Roh M S, Kahky M P, Oyedeji C, Klostergaard J, Wang L, Curley S A and Lotzova E. Murine Kupffer cells and hepatic natural killer cells regulate tumor growth in a quantitative model of colorectal liver metastases. Clinical & experimental metastasis. 1992; 10(5):317-327.
39. Matsumura H, Kondo T, Ogawa K, Tamura T, Fukunaga K, Murata S and Ohkohchi N. Kupffer cells decrease metastasis of colon cancer cells to the liver in the early stage. International journal of oncology. 2014; 45(6):2303-2310.
40. Miyagawa S, Miwa S, Soeda J, Kobayashi A and Kawasaki S. Morphometric analysis of liver macrophages in patients with colorectal liver metastasis. Clinical & experimental metastasis. 2002; 19(2):119-125.
41. Van den Eynden G G, Majeed AW, Illemann M, Vermeulen P B, Bird N C, Hoyer-Hansen G, Eefsen R L, Reynolds A R and Brodt P. The multifaceted role of the microenvironment in liver metastasis: biology and clinical implications. Cancer research. 2013; 73(7):2031-2043.
42. Kruse J, von Bernstorff W, Evert K, Albers N, Hadlich S, Hagemann S, Gunther C, van Rooijen N, Heidecke C D and Partecke L I. Macrophages promote tumour growth and liver metastasis in an orthotopic syngeneic mouse model of colon cancer. International journal of colorectal disease. 2013; 28(10):1337-1349.
43. Parker G A and Picut C A. Immune functioning in non lymphoid organs: the liver. Toxicologic pathology. 2012; 40(2):237-247.
44. Miyamoto M, Emoto M, Brinkmann V, van Rooijen N, Schmits R, Kita E and Kaufmann S H. Cutting edge: contribution of NK cells to the homing of thymic CD4+NKT cells to the liver. Journal of immunology. 2000; 165(4):1729-1732.
45. Seki S, Nakashima H, Nakashima M and Kinoshita M. Antitumor immunity produced by the liver Kupffer cells, NK cells, NKT cells, and CD8 CD122 T cells. Clinical & developmental immunology. 2011; 2011:868345.
46. Seki S, Habu Y, Kawamura T, Takeda K, Dobashi H, Ohkawa T and Hiraide H. The liver as a crucial organ in the first line of host defense: the roles of Kupffer cells, natural killer (NK) cells and NK1.1 Ag+ T cells in T helper 1 immune responses. Immunological reviews. 2000; 174:35-46.
47. Jha A K, Huang S C, Sergushichev A, Lampropoulou V, Ivanova Y, Loginicheva E, Chmielewski K, Stewart K M, Ashall J, Everts B, Pearce E J, Driggers E M and Artyomov M N. Network integration of parallel metabolic and transcriptional data reveals metabolic modules that regulate macrophage polarization. Immunity. 2015; 42(3):419-430.
48. Murray P J, Allen J E, Biswas S K, Fisher E A, Gilroy D W, Goerdt S, Gordon S, Hamilton J A, Ivashkiv L B, Lawrence T, Locati M, Mantovani A, Martinez F O, Mege J L, Mosser D M, Natoli G, et al. Macrophage activation and polarization: nomenclature and experimental guidelines. Immunity. 2014; 41(1):14-20.
49. Xue J, Schmidt S V, Sander J, Draffehn A, Krebs W, Quester I, De Nardo D, Gohel T D, Emde M, Schmidleithner L, Ganesan H, Nino-Castro A, Mallmann M R, Labzin L, Theis H, Kraut M, et al. Transcriptome-based network analysis reveals a spectrum model of human macrophage activation. Immunity. 2014; 40(2):274-288.
50. Hao N B, Lu M H, Fan Y H, Cao Y L, Zhang Z R and Yang S M. Macrophages in tumor microenvironments and the progression of tumors. Clinical & developmental immunology. 2012; 2012:948098.
51. Standiford T J, Kuick R, Bhan U, Chen J, Newstead M and Keshamouni V G. TGF-beta-induced IRAK-M expression in tumor-associated macrophages regulates lung tumor growth. Oncogene. 2011; 30(21):2475-2484.
52. Umemura N, Saio M, Suwa T, Kitoh Y, Bai J, Nonaka K, Ouyang G F, Okada M, Balazs M, Adany R, Shibata T and Takami T. Tumor-infiltrating myeloid-derived suppressor cells are pleiotropic-inflamed monocytes/macrophages that bear Ml- and M2-type characteristics. Journal of leukocyte biology. 2008; 83(5):1136-1144.
53. Bastos K R, Alvarez J M, Marinho C R, Rizzo L V and Lima M R. Macrophages from IL-12p40-deficient mice have a bias toward the M2 activation profile. Journal of leukocyte biology. 2002; 71(2):271-278.
54. Ryan A E, Colleran A, O′Gorman A, O′Flynn L, Pindjacova J, Lohan P, O′Malley G, Nosov M, Mureau C and Egan L J. Targeting colon cancer cell NF-kappaB promotes an anti-tumour M1-like macrophage phenotype and inhibits peritoneal metastasis. Oncogene. 2015; 34(12):1563-1574.
55. Santoni M, Massari F, Amantini C, Nabissi M, Maines F, Burattini L, Berardi R, Santoni G, Montironi R, Tortora G and Cascinu S. Emerging role of tumor-associated macrophages as therapeutic targets in patients with metastatic renal cell carcinoma. Cancer immunology, immunotherapy : CII. 2013; 62(12):1757-1768.
56. Costa N L, Valadares M C, Souza P P, Mendonca E F, Oliveira J C, Silva T A and Batista A C. Tumor-associated macrophages and the profile of inflammatory cytokines in oral squamous cell carcinoma. Oral oncology. 2013; 49(3):216-223.
57. Heusinkveld M, de Vos van Steenwijk PJ, Goedemans R, Ramwadhdoebe T H, Gorter A, Welters M J, van Hall T and van der Burg S H. M2 macrophages induced by prostaglandin E2 and IL-6 from cervical carcinoma are switched to activated M1 macrophages by CD4+Th1 cells. Journal of immunology. 2011; 187(3):1157-1165.
58. Duluc D, Corvaisier M, Blanchard S, Catala L, Descamps P, Gamelin E, Ponsoda S, Delneste Y, Hebbar M and Jeannin P. Interferon-gamma reverses the immunosuppressive and protumoral properties and prevents the generation of human tumor-associated macrophages. International journal of cancer Journal international du cancer. 2009; 125(2):367-373.
59. Hata A and Lieberman J. Dysregulation of microRNA biogenesis and gene silencing in cancer. Science signaling. 2015; 8(368):re3.
60. Yang F, Zhang W, Shen Y and Guan X. Identification of dysregulated microRNAs in triple-negative breast cancer (review). International journal of oncology. 2015; 46(3):927-932.
61. Joshi P, Middleton J, Jeon Y J and Garofalo M. MicroRNAs in lung cancer. World journal of methodology. 2014; 4(2):59-72.
62. Oom A L, Humphries B A and Yang C. MicroRNAs: novel players in cancer diagnosis and therapies. BioMed research international. 2014; 2014:959461.
63. Cho W C. MicroRNAs as therapeutic targets and their potential applications in cancer therapy. Expert opinion on therapeutic targets. 2012; 16(8):747-759.
64. Parasramka M A, Ho E, Williams D E and Dashwood R H. MicroRNAs, diet, and cancer: new mechanistic insights on the epigenetic actions of phytochemicals. Molecular carcinogenesis. 2012; 51(3):213-230.
65. Wang Z, Brandt S, Medeiros A, Wang S, Wu H, Dent A and Serezani C H. MicroRNA 21 is a homeostatic regulator of macrophage polarization and prevents prostaglandin E2-mediated M2 generation. PloS one. 2015; 10(2):e0115855.
66. Caescu C I, Guo X, Tesfa L, Bhagat T D, Verma A, Zheng D and Stanley E R. Colony stimulating factor-1 receptor signaling networks inhibit mouse macrophage inflammatory responses by induction of microRNA-21. Blood. 2015; 125(8):e1-13.
67. Xu S, Wei J, Wang F, Kong L Y, Ling X Y, Nduom E, Gabrusiewicz K, Doucette T, Yang Y, Yaghi N K, Fajt V, Levine J M, Qiao W, Li X G, Lang F F, Rao G, et al. Effect of miR-142-3p on the M2 macrophage and therapeutic efficacy against murine glioblastoma. Journal of the National Cancer Institute. 2014; 106(8).
68. Veremeyko T, Siddiqui S, Sotnikov I, Yung A and Ponomarev E D. IL-4/IL-13-dependent and independent expression of miR-124 and its contribution to M2 phenotype of monocytic cells in normal conditions and during allergic inflammation. PloS one. 2013; 8(12):e81774.
69. Zhou D, Huang C, Lin Z, Zhan S, Kong L, Fang C and Li J. Macrophage polarization and function with emphasis on the evolving roles of coordinated regulation of cellular signaling pathways. Cellular signalling. 2014; 26(2):192-197.
70. Moore C S, Rao V T, Durafourt B A, Bedell B J, Ludwin S K, Bar-Or A and Antel J P. miR-155 as a multiple sclerosis-relevant regulator of myeloid cell polarization. Annals of neurology. 2013; 74(5):709-720.
71. Jenne C N and Kubes P. Immune surveillance by the liver. Nature immunology. 2013; 14(10):996-1006.
72. Pellicoro A, Ramachandran P, Iredale J P and Fallowfield J A. Liver fibrosis and repair: immune regulation of wound healing in a solid organ. Nature reviews Immunology. 2014; 14(3):181-194.
73. Bilzer M, Roggel F and Gerbes A L. Role of Kupffer cells in host defense and liver disease. Liver international : official journal of the International Association for the Study of the Liver. 2006; 26(10):1175-1186.
74. Wang Q, Ren Y, Mu J, Egilmez N K, Zhuang X, Deng Z, Zhang L, Yan J, Miller D and Zhang H G. Grapefruit-derived nanovectors use an activated leukocyte trafficking pathway to deliver therapeutic agents to inflammatory tumor sites. Cancer research. 2015.
75. Wang Q, Zhuang X, Mu J, Deng Z B, Jiang H, Zhang L, Xiang X, Wang B, Yan J, Miller D and Zhang H G. Delivery of therapeutic agents by nanoparticles made of grapefruit-derived lipids. Nature communications. 2013; 4:1867.
76. Humphreys K J, McKinnon R A and Michael M Z. miR-18a inhibits CDC42 and plays a tumour suppressor role in colorectal cancer cells. PloS one. 2014; 9(11):e112288.
77. Jiang H, Wang P, Wang Q, Wang B, Mu J, Zhuang X, Zhang L, Yan J, Miller D and Zhang H G. Quantitatively controlling expression of miR-1792 determines colon tumor progression in a mouse tumor model. The American journal of pathology. 2014; 184(5):1355-1368.
78. Leonard J P, Sherman M L, Fisher G L, Buchanan L J, Larsen G, Atkins M B, Sosman J A, Dutcher J P, Vogelzang N J and Ryan J L. Effects of single-dose interleukin-12 exposure on interleukin-12-associated toxicity and interferon-gamma production. Blood. 1997; 90(7):2541-2548.
79. Liu W H, Yeh S H, Lu C C, Yu S L, Chen H Y, Lin C Y, Chen D S and Chen P J. MicroRNA-18a prevents estrogen receptor-alpha expression, promoting proliferation of hepatocellular carcinoma cells. Gastroenterology. 2009; 136(2):683-693.

It will be understood that various details of the presently-disclosed subject matter can be changed without departing from the scope of the subject matter disclosed herein. Furthermore, the foregoing description is for the purpose of illustration only, and not for the purpose of limitation.

What is claimed is:

1. A composition, comprising a miRNA encapsulated by a microvesicle, wherein the microvesicle is derived from an edible plant.

2. The composition of claim 1, wherein the edible plant is a fruit.

3. The composition of claim 2, wherein the fruit is selected from a grape, a grapefruit, and a tomato.

4. The composition of claim 1, wherein the miRNA is selected from miR18a and miR17.

5. The composition of claim 1, wherein the microvesicle comprises a cancer targeting moiety for directing the composition to a cancer cell.

6. The composition of claim 5, wherein the cancer targeting moiety comprises folic acid.

7. The composition of claim 1, wherein the microvesicle comprises a nanovector hyrided with polyethylenimine.

8. The composition of claim 7, wherein the nanovector comprises a grapefruit-derived nanovector.

9. The composition of claim 7, wherein the polyethylenimine increases a miRNA carrying capacity of the nanovector.

10. The composition of claim 7, wherein the nanovector decreases a toxicity of the polyethylenimine.

11. A pharmaceutical composition, comprising:
a microvesicle;
a miRNA encapsulated by the microvesicle; and
a pharmaceutically-acceptable vehicle, carrier, or excipient;
wherein the microvesicle is derived from an edible plant.

12. The pharmaceutical composition of claim 11, wherein the edible plant is a fruit.

13. The composition of claim 12, wherein the fruit is selected from a grape, a grapefruit, and a tomato.

14. The composition of claim 11, wherein the miRNA is selected from miR18a and miR17.

15. The composition of claim 11, wherein the microvesicle comprises a cancer targeting moiety for directing the composition to a cancer cell.

16. The composition of claim 15, wherein the cancer targeting moiety comprises folic acid.

17. A method for treating a cancer in a subject, comprising administering to a subject an effective amount of a composition comprising a miRNA encapsulated by a microvesicle, wherein the microvesicle is derived from an edible plant.

18. The method of claim 17, wherein the cancer is selected from a brain cancer, a liver cancer, and a colon cancer.

19. The method of claim 17, wherein the cancer is a liver metastases.

20. The method of claim 17, wherein administering the composition to the subject comprises orally or intranasally administering the composition to the subject.
